(19) United States
(12) Patent Application Publication
(10) Pub. No.: US 2026/0335274 A1
(43) Pub. Date: Oct. 29, 2026

(54) COMPOSITIONS AND METHODS OF MINIMIZING LONG COVID

(71) Applicant: AUGUSTA UNIVERSITY RESEARCH INSTITUTE, INC., Augusta, GA (US)

(72) Inventor: Stephen HSU, Augusta, GA (US)

(21) Appl. No.: 19/111,317

(22) PCT Filed: Sep. 15, 2023
(86) PCT No.: PCT/US2023/074377

§ 371 Date: Mar. 13, 2025

Related U.S. Application Data
(60) Provisional application No. 63/407,464, filed on Sep. 16, 2022.
(60) Provisional application No. 63/490,712, filed on Mar. 16, 2023.

(51) Int. Cl. A61K 31/353; A61K 9/00; A61K 9/10; A61K 9/51; A61K 36/82; A61M 15/00; A61P 31/14

(52) U.S. Cl. CPC A61K 31/353; A61K 9/0043; A61K 9/008; A61K 9/10; A61K 9/5115; A61K 9/5161; A61K 36/82; A61M 15/0065; A61P 31/14

(57) ABSTRACT

Pharmaceutical formulations for nasal administration are disclosed. The pharmaceutical formulation contains a green tea catechin derivative (such as an epigallocatechin-3-gallate-palmitate); and preferably further includes a carbohydrate (such as a polysaccharide); and a pharmaceutically acceptable carrier. The pharmaceutical formulation has a viscosity sufficient to maintain the green tea catechin derivative in contact with nasal epithelial cells optionally for at least about 30 minutes in the presence of mucus. Also described are methods of using the nasal formulations for preventing, reducing, and/or treating neurological damages caused by exposure to a virus, such as a SARS-CoV-2. For example, the disclosed pharmaceutical formulations can treat or prevent one or more symptoms of long COVID in an infected subject, and/or repair the damaged nasal epithelial cells, neuroepithelial cells, vascular cells, and/or neurons and restore their functions in a subject recovered from a COVID infection, such as the olfactory function of these cells.

### CROSS-REFERENCE TO RELATED APPLICATION

This application claims benefit and priority to U.S. Application No. 63/407,464, filed Sep. 16, 2022, and U.S. Application No. 63/490,712 filed Mar. 16, 2023, the disclosures of which are incorporated herein by reference.

### STATEMENT REGARDING FEDERALLY SPONSORED RESEARCH OR DEVELOPMENT

This invention was made with government support under R41DC020678 awarded by The National Institute of Health. The government has certain rights in the invention.

### REFERENCE TO SEQUENCE LISTING

The Sequence Listing submitted as a text file named “AURI_2023_002_PCT.xml”, created on Sep. 15, 2023, and having a size of 3,067 bytes is hereby incorporated by reference pursuant to 37 C.F.R. § 1.52 (e) (5).

### FIELD OF THE INVENTION

This invention is generally in the field of compositions and methods of use thereof for the prevention and/or treatment of neurological damage caused by exposure to virus.

### BACKGROUND OF THE INVENTION

Infection with severe acute respiratory syndrome coronavirus 2 (SARS-CoV-2) is associated with high mortality and morbidity worldwide. A common sequela is chronic neurologic diseases, which severely impact the quality of life and increase the burden on healthcare systems. About 50% of patients infected by SARS-CoV-2 experience chronic post COVID-19 symptoms (also referred to as Long COVID), including nervous system and neurocognitive disorders, which include headache, persistent loss of smell and/or taste, memory loss, brain fog (difficulty concentrating, sense of confusion or disorientation), dizziness, anxiety, depression, earache, hearing loss, and/or ringing in ears (tinnitus). The Long COVID neurologic symptoms are due to the robust replication of SARS-CoV-2 in the nasal neuroepithelial cells, leading to neuro-invasion and inflammation of the central nerve system (CNS). In addition, chronic anosmia associated with Long COVID is due to the “persistent infection” of SARS-CoV-2 in the olfactory epithelium, leading to inflammation, apoptosis, and damage to the cells responsible for smell functions. Currently used medications and vaccines do not inhibit the robust SARS-CoV-2 replication in the nasal epithelial cells, nor the persistent infection of the virus in the olfactory epithelium. For example, neutralizing antibodies, either injected in the nasal cavity or acquired via vaccination, are not effective at reducing the SARS-CoV-2 viral load in the nasal cavity due to robust viral replication in the nasal turbinates, and clinical trial data showed that nasal spray of steroids failed to restore olfactory function due to the persistent viral infection. Additionally, asymptomatic patients have a nasal viral load comparable to symptomatic patients, indicating that both symptomatic and asymptomatic patients are at risk for anosmia.

There is an urgent need for treatment/preventive measures for rapidly inhibiting viral replication, such as SARS-CoV-2 replication, in the nasal cavity to block viral invasion to CNS and clear the persistent infection in order to reduce/prevent neurologic damages.

Therefore, it is an object of the present invention to provide pharmaceutical formulations for nasal administration, which can prevent, reduce, minimize, and/or treat neurological damage caused by exposure to virus, such as SARS-CoV-2.

It is a further object of the present invention to provide methods for preventing or reducing neurological damage, such as olfactory dysfunction, caused by exposure to a virus, such as SARS-CoV-2, in a subject in need thereof.

It is a further object of the present invention to provide methods for repairing nasal epithelial cells, neuroepithelial cells, olfactory sensory neurons, vascular cells, and/or neurons damaged by a virus, such as SARS-CoV-2, in a subject in need thereof.

### SUMMARY OF THE INVENTION

Described are pharmaceutical formulations for nasal administration (also referred herein as “nasal formulations”). Also described are methods of using the nasal formulations for preventing, reducing, minimizing, and/or treating neurological damage caused by exposure to virus, such as a respiratory virus including, but not limited to, an influenza virus (e.g., influenza A, influenza virus B, or influenza virus C), respiratory syncytial virus (RSV), human metapneumovirus, or a coronaviruses such as SARS-CoV-1 or SARS-CoV-2.

Without being bound to any theories, it is believed that the disclosed pharmaceutical formulations can inhibit or reduce viral replication in nasal epithelia and/or neuroinvasion of CNS, and thereby prevent or reduce neurological damage, such as those caused by exposure to a respiratory virus such as SARS-CoV-2. It is also believed that the disclosed pharmaceutical formulations can promote cell differentiation, reduce inflammation/apoptosis, and/or reduce oxidative stress of nasal epithelial cells, neuroepithelial cells, vascular cells, and/or neurons caused by exposure to virus, and thereby repair the damaged nasal epithelial cells, neuroepithelial cells, vascular cells, and/or neurons and restore their functions. For example, it is believed that by promoting cell differentiation, reducing inflammation, and/or reducing oxidative stress of nasal epithelial cells caused by exposure to a respiratory virus such as SARS-CoV-2, the disclosed pharmaceutical formulations can restore the olfactory function of these cells.

The pharmaceutical formulation contains a green tea catechin derivative; and optionally a carbohydrate, such as a sugar alcohol or a saccharide (including monosaccharides, disaccharides, oligosaccharides, and polysaccharides); and a pharmaceutically acceptable carrier. Typically, the pharmaceutical formulation has a viscosity sufficient to maintain the green tea catechin derivative in contact with nasal epithelial cells for an effective amount of time to have a therapeutic effect and optionally without causing a stuffy nose. In some embodiments, the amount of time is at least about 30 minutes in the presence of mucus, such as from 30 mins to 4 hours. For example, the pharmaceutical formulation has a viscosity of at least about 15 cps, at least about 20 cps, at least about 25 cps, or at least about 30 cps and up to about 150 cps, such as in a range from about 15 cps to about 150 cps, from about 15 cps to about 125 cps, from about 15 cps to about 150 cps, from about 15 cps to about 95 cps, from about 15 cps to about 75 cps, from about 15 cps to about 65 cps, from about 20 cps to about 65 cps, from about 25 cps to about 65 cps, from about 30 cps to about 65 cps, from about 35 cps to about 65 cps, from about 40 cps to about 65 cps, from about 45 cps to about 65 cps, from about 30 cps to about 60 cps, from about 35 cps to about 60 cps, from about 40 cps to about 60 cps, from about 35 cps to about 55 cps, from about 40 cps to about 55 cps, or from about 45 cps to about 55 cps, such as about 50 cps.

In some forms, the green tea catechin derivative of the pharmaceutical formulation contains a fatty/aromatic acyl group, such as a long chain aliphatic acyl group (i.e., having an aliphatic group with at least five carbon atoms, such as about 13-21 carbon atoms), which stabilizes the catechin moiety from auto-oxidation and makes the green tea catechin derivative lipid-soluble, and thereby allows the green tea catechin derivative to attach to the cell membrane at the delivery site for a longer period of time compared to its counterpart without the fatty/aromatic acyl group, to perform antiviral, anti-inflammatory, and/or anti-oxidative actions, and thereby inhibit or reduce viral replication in nasal epithelia and/or neuroinvasion of CNS, and/or repair damaged nasal epithelial cells, neuroepithelial cells, vascular cells, and/or neurons and restore their functions. For example, a long chain fatty acyl group containing epigallocatechin-3-gallate (“EGCG”) derivative, such as an EGCG palmitate, can attach to the cell membrane for a longer period of time compared to EGCG, following nasal administration to nasal epithelial cells. The green tea catechin derivative of the disclosed pharmaceutical formulation is also safe, i.e., does not show cytotoxicity to normal living cells. For example, the use of green tea polyphenol palmitates (contains 50% EGCG palmitate) was approved by the FDA as a GRAS dietary additive (FDA GRAS Notice 772).

In some forms, the green tea catechin derivative of the disclosed pharmaceutical formulation is a derivative of epigallocatechin-3-gallate, epicatechin, epigallocatechin, or epicatechin-3-gallate, or a combination thereof. For example, in some forms, the green tea catechin derivative is represented by Formula I.

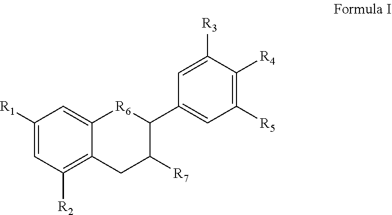

wherein: (i) R₁-R₅ and R₇ can be independently hydrogen, hydroxyl,

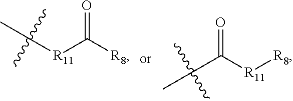

R₈ can be a substituted C₁-C₃₀ alkyl, an unsubstituted C₁-C₃₀ alkyl, a substituted C₂-C₃₀ alkenyl, an unsubstituted C₂-C₃₀ alkenyl, a substituted C₂-C₃₀ alkynyl, an unsubstituted C₂-C₃₀ alkynyl, a substituted aryl, an unsubstituted aryl, a substituted polyaryl, or an unsubstituted polyaryl; (ii) R₆ and R₁₁ can be independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; and (iii) at least one of R₁-R₅ and R₇ can be

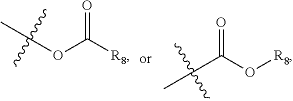

or a pharmaceutically acceptable salt thereof.

In some forms, the green tea catechin derivative can be represented by Formula II:

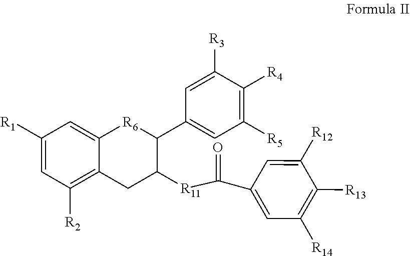

wherein: (i) R₁-R₅ and R₁₂-R₁₄ can be independently hydrogen, hydroxyl,

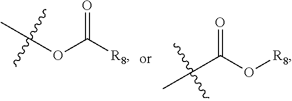

R₈ can be a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; (ii) R₆ and R₁₁ can be independently oxygen, —NR₉R₁₀, or sulfur, R₉ and R₁₀ can be independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, or an unsubstituted C₁-C₃₀ alkynyl; and (iii) least one of R₁-R₅ and R₁₂-R₁₄ can be

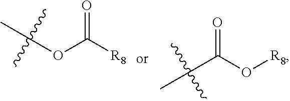

or a pharmaceutically acceptable salt thereof.

In some forms of Formula II, wherein R₆ and/or R₁₁ are oxygen. In some forms of Formula I and/or II, wherein R₁ and R₂ are hydroxyl. In some forms of Formula I and/or II, at least one of R₃-R₅ is

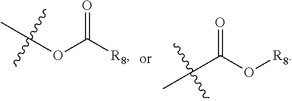

In some forms of Formula II, R₁₂-R₁₄ are independently hydroxyl,

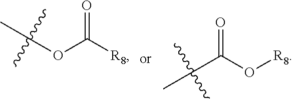

In some forms of Formula I and/or II, R₈ is a linear or branched substituted C₁-C₃₀ alkyl or a linear or branched unsubstituted C₁-C₃₀ alkyl, such as a linear or branched substituted C₁₄-C₂₅ alkyl or a linear or branched unsubstituted C₁₄-C₂₅ alkyl, for example, C₁₅H₃₁.

In some forms, the green tea catechin derivative of the pharmaceutical formulation can be an epigallocatechin-3-gallate-palmitate. For example, the green tea catechin derivative has the structure of:

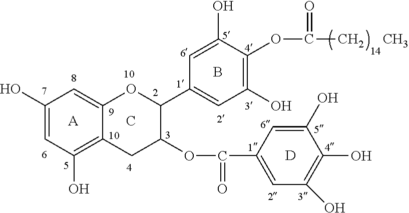

The carbohydrate can be a sugar alcohol or a saccharide. In some forms, the carbohydrate is a sugar alcohol. In some forms, the carbohydrate is a saccharide, such as a monosaccharide, disaccharide, oligosaccharide, or a polysaccharide, or a combination thereof. In some forms, the saccharide is a polysaccharide, such as a cellulose (e.g., sodium carboxymethyl cellulose), a derivative thereof, or a salt thereof, or a combination thereof.

Typically, the green tea catechin derivative is present in the pharmaceutical formulation in an amount effective to inhibit or reduce viral replication in nasal epithelia and/or neuroinvasion of CNS, and/or repair damaged nasal epithelial cells, neuroepithelial cells, vascular cells, and/or neurons and restore their functions in a subject in need thereof. For example, the amount of green tea catechin derivative can be from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation.

In some forms, the carbohydrate, such as a polysaccharide, of the pharmaceutical formulation can increase the solubility of the green tea catechin derivative and/or adjust the viscosity of the pharmaceutical formation to enhance the attachment of the green tea catechin derivate to cell membranes such that the duration of the green tea catechin derivative in contact with nasal epithelial cells for a therapeutically effective amount of time, optionally but preferably at least 30 minutes, in the presence of mucus.

In some forms, the carbohydrate is a sugar alcohol, such as glycerol. In some forms, the carbohydrate is a saccharide, such as a monosaccharide, disaccharide, oligosaccharide, or a polysaccharide, or a combination thereof. In some forms, the carbohydrate is a polysaccharide, such as a cellulose, a complex polysaccharide, a derivative thereof, or a salt thereof, or a combination thereof. In some forms, the polysaccharide of the pharmaceutical formulation can be one or more of carboxymethylcellulose sodium, microcrystalline cellulose, xyloglucan, hypomellose, glyceryl polymethacrylate, xanthan gum, hydroxyethylcellulose, guar gum, locust bean gum, carboxymethylcellulose, and hydroxypropylmethylcellulose. For example, in some forms, the polysaccharide of the pharmaceutical formulation is xyloglucan, glyceryl polymethacrylate, xanthan gum, hydroxyethylcellulose, carboxymethylcellulose sodium, hypomellose, or microcrystalline cellulose, or a combination thereof, such as xyloglucan.

Typically, the carbohydrate, such as a polysaccharide, is present in the pharmaceutical formulation in an amount to provide a suitable viscosity for the pharmaceutical formulation such that the green tea catechin derivative remains in contact with nasal epithelial cells for example, for at least 30 minutes in the presence of mucus. For example, the amount of carbohydrate, such as the polysaccharide, is from about 0.01% (w/v) to about 8% (w/v), from about 0.1% (w/v) to about 8% (w/v), from about 0.5% (w/v) to about 8% (w/v), from about 1% (w/v) to about 8% (w/v), from about 2% (w/v) to about 8% (w/v), from about 0.01% (w/v) to about 6% (w/v), from about 0.1% (w/v) to about 6% (w/v), from about 0.5% to about 6% (w/v), from about 1% to about 6% (w/v), from about 2% to about 6% (w/v), from about 0.01% (w/v) to about 4% (w/v), from about 0.1% (w/v) to about 4% (w/v), from about 0.5% to about 4% (w/v), from about 1% to about 4% (w/v), or from about 2% (w/v) to about 4% (w/v) of the pharmaceutical formulation. The precise amount of carbohydrate in the pharmaceutical formulation can vary according to a variety of factors such as subject-dependent variables (e.g., age, immune system health, etc.), the disease or disorder being treated, as well as the route of administration and the pharmacokinetics of the agent being administered.

The pharmaceutical formulation may further contain one or more pharmaceutically acceptable excipients, such as an emulsifier, an emollient, a buffering agent, a thickening agent, a chelating agent, or a preservative, or a combination thereof, for example, one or more of polysorbate 80, glycerin, eucalyptol, propolene glycol, povidone, and benzalkonium chloride.

The pharmaceutical formulation may contain one or more active agents, in addition to the green tea catechin derivative, such as an anti-inflammatory agent, an antioxidant, or antiviral agent, or a combination thereof.

The disclosed pharmaceutical formulation is typically in a liquid form, such as an emulsion or a suspension. The suspension can be an aqueous or minimally aqueous suspension. A minimally aqueous suspension typically contains less than 10 wt % of water in the formulation. Typically, the liquid pharmaceutical formulation has a pH in a range from 3 to 7.4, from 4 to 7, from 4 to 6, from 4 to 5, from 5 to 7, or from 5 to 6, such as about 6 and can be isotonic or hypertonic.

When the disclosed pharmaceutical formulation is an emulsion (also referred to herein as “emulsion formulation”), such as a nanoemulsion, at least two pharmaceutically acceptable carriers and optionally an emulsifier, can be used for forming the emulsion. For example, the pharmaceutical formulation can contain a first pharmaceutically acceptable carrier that is an aqueous solution or water (such as distilled water, deionized water, and/or tap water), and a second pharmaceutically acceptable carrier that is an oil (such as an animal or vegetable oil). The emulsion can be a water-in-oil emulsion, an oil-in-water emulsion, a water-in-oil-in-water emulsion, or an oil-in-water-in-oil emulsion. Optionally, the pharmaceutical formulation further contains a third pharmaceutically acceptable carrier that is an organic solvent, such as DMSO or an alcohol (e.g., ethanol).

In some forms, the disclosed pharmaceutical formulation is an oil-in-water emulsion, wherein the oil forms oil droplets dispersed in the aqueous solution or water. Generally, the amount of oil forming the oil droplets in the emulsion can be present in an amount up to about 10 vol % or up to about 5 vol %, such as in a range from about 0.1 vol % to about 10 vol %, from about 0.5 vol % to about 10 vol %, from about 1 vol % to about 10 vol %, from about 0.1 vol % to about 5 vol %, from about 0.5 vol % to about 5 vol %, from about 1 vol % to about 5 vol %, from about 0.1 vol % to about 4 vol %, from about 0.5 vol % to about 4 vol %, from about 1 vol % to about 4 vol %, or from about 0.1 vol % to about 2 vol % of the pharmaceutical formulation. Generally, the oil droplets of the emulsion can have an average diameter of less than 1 micron, less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 150 nm, less than or equal to about 100 nm, or less than or equal to about 50 nm, such as in a range from about 100 nm to about 600 nm, from about 150 nm to about 600 nm, from about 200 nm to about 600 nm, from about 250 nm to about 600 nm, from about 300 nm to about 600 nm, from about 300 nm to about 500 nm, or from about 300 nm to about 400 nm. In these forms, the green tea catechin derivative of the pharmaceutical formulation is encapsulated in the oil droplets.

In some forms, the disclosed pharmaceutical formulation is a suspension (also referred to herein as “suspension formulation”). The suspension can be an aqueous or minimally aqueous suspension. Typically, the pharmaceutically acceptable carrier of the suspension formulation contains glycerol or a liquid poly(ethylene glycol), or a combination thereof; and optionally one or more of the following: an alcohol (e.g., methanol, ethanol, n-propanol, isopropanol, etc.), water, and an aqueous solution (e.g., normal saline, phosphate buffered saline, etc.). A liquid poly(ethylene glycol) typically has a molecular weight of ≤600 Da. When an alcohol is present in the suspension formulation, the alcohol is typically in a trace amount, i.e., ≤1 wt %. The specific amount of each pharmaceutically acceptable carrier in the suspension formulation depends on the desired concentration of the green tea catechin derivative, the specific solvent, etc. In these forms, the green tea catechin derivative of the pharmaceutical formulation is in the form of particles, such as nanoparticles. These green tea catechin derivative particles can have an average diameter of <5 μm, <4 μm, <3.5 μm, <3 μm, <2.5 μm, or <2 μm. For example, the green tea catechin derivative particles in the disclosed pharmaceutical formulation, in the form of a suspension, have a median diameter of less than 500 nm, less than 400 nm, less than 300 nm, or less than 200 nm, such as ranging from about 10 nm to about 500 nm, from about 10 nm to about 400 nm, from about 10 nm to about 300 nm, from about 10 nm to about 200 nm, from about 10 nm to about 150 nm, from about 50 nm to about 500 nm, from about 50 nm to about 400 nm, from about 50 nm to about 300 nm, from about 50 nm to about 200 nm, or from about 50 nm to about 150 nm. In some forms, the green tea catechin derivative particles of the pharmaceutical formulation have a fine particle fraction of >40%, >45%, >50%, >55%, or >60%.

Delivery devices and kits including one or more of the pharmaceutical formulations are also disclosed. Exemplary delivery devices include dropper and spray bottles and inhalers filled with the disclosed pharmaceutical formulation. The inhaler can be any inhaler suitable for delivering a liquid formulation. In a particular example, the device is a pressurized metered dose inhaler (i.e., a nebulizer) that is configured to deliver a unit dosage of the pharmaceutical formulation per puff. An exemplary kit can include one or more nasal swabs (such as sterile nasal swabs) and the disclosed pharmaceutical formulation. The nasal swab and the pharmaceutical formulation can be packaged separately, or the nasal swab can be impregnated or saturated with the pharmaceutical formulation.

Methods for using the disclosed pharmaceutical formulations are also disclosed. For example, the disclosed pharmaceutical formulations can be used for preventing or reducing neurological damage caused by exposure to a virus, for repairing nasal epithelial cells, neuroepithelial cells, vascular cells, and/or neurons damaged by a virus, and/or for treating one or more symptoms of long COVID including, but not limited to, anosmia, in a subject in need thereof.

Generally, the method for preventing or reducing neurological damage caused by exposure to a virus, particularly a respiratory virus such as SARS-CoV-2 or a variant thereof (e.g., B.1.617.2 (Delta) and/or B.1.1.529 (Omicron)), in a subject in need thereof, includes (i) administering the disclosed pharmaceutical formulation to the nasal vestibule or passages of the subject. The administration step can be performed before and/or after the subject is exposed to the virus. For example, the subject is a mammal who was exposed, is exposed to, and/or is expected to be exposed to an individual having one or more symptoms of a viral infection, such as with fever, cough, shortness of breath, diarrhea, sneezing, runny nose, and/or sore throat. The administration step may be repeated once, twice, or three times, per day, for a period of one day, three days, one week, two weeks, one month, three months, six months, or one year. Typically, following the administration or all of the administrations of the disclosed pharmaceutical formulation, the subject shows no or less symptoms associated with neurological damages caused by exposure to the virus, such as headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, or gastrointestinal issues, or a combination thereof, compared to a control. A “control” refers to a subject administered with a nasal formulation without the green tea catechin derivative, such as a normal saline nasal formulation.

Generally, the method for repairing nasal epithelial cells, neuroepithelial cells, vascular cells, and/or neurons damaged by a virus, particularly a respiratory virus such as SARS-CoV-2 or a variant thereof (e.g., B.1.617.2 (Delta) and/or B.1.1.529 (Omicron)), in a subject in need thereof, includes (i) administering the disclosed pharmaceutical formulation to the nasal vestibule or passages of the subject. The administration step can be performed after the subject is recovered from a viral infection, as indicated by a negative test result for the virus. Although recovered from the viral infection, the subject shows one or more symptoms associated with neurological damages caused by exposure to the virus, such as one or more of headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues. The administration step may be repeated once, twice, or three times, per day, for a period of one day, three days, one week, two weeks, one month, three months, six months, or one year. Typically, following the administration or all of the administrations of the disclosed pharmaceutical formulation, the subject shows less or none of the symptoms associated with neurological damages caused by exposure to the virus, compared to the subject prior to the administration or first administration and/or compared to a similarly exposed untreated subject.

### BRIEF DESCRIPTION OF THE FIGURES

FIGS. 1A-1D are light microscope images of F18 stock (FIG. 1A); F18MM (FIG. 1B); F18 Saline (FIG. 1C); F18 PBS (FIG. 1D). FIG. 1E is a graph showing the size distribution of particles in saline-diluted F18 (one representative sample determined by NTA).

FIG. 2 is a graph showing the dose response of antiviral activity of F18 EC16 nasal formulation. The formulations were diluted from an F18 glycerol stock of EC16 (1%) into serum-free EMEM to a concentration of 0.05, 0.125, 0.25, 0.625, and 1.25 mM, incubated with OC43 (HCoV-OC43) virus at a 1:9 ratio (virus to formulation) for 30 min prior to a series of 10× dilutions, and then subjected to TCID50 assay. The contact inhibition antiviral activity was calculated and expressed as log₁₀ reduction with standard deviation. Results are from three independent experiments.

FIG. 3 is a graph showing the time response of contact inhibition antiviral activity of F18 EC16 nasal formulation. The formulation was diluted from an F18 glycerol stock of EC16 (1%) in serum-free EMEM, to a concentration of 1.25 mM EC16. This formulation was incubated with OC43 virus at a 1:9 ratio (virus to formulation) for 5, 15, 30, and 60 min before 10× serial dilutions, and then subjected to TCID50 assay. The antiviral activity was calculated and expressed as log₁₀ reduction with standard deviation. Results are from three independent experiments.

FIG. 4 is a bar graph showing contact inhibition antiviral activity of F18 EC16 suspensions in three diluents. The glycerol-based F18 EC16 nasal formulation stock (1%) was diluted 10× with serum-free EMEM (MM), normal saline, or phosphate buffered saline (PBS) to 1.25 mM. The suspensions were incubated with OC43 virus for 30 min at a 1:9 ratio (virus: formulation) prior to serial 10× dilutions, and then subjected to TCID50 assay. Results were obtained from three independent experiments with standard deviation (the saline test result values were identical).

FIG. 5 is a bar graph showing antiviral activities of EC16m and Remdesivir using post-infection tests with OC43 virus. Results were obtained from four independent experiments.

FIG. 6 is a graph showing the dose response of antiviral activity of F18m (EC16m) nasal formulation. The formulations were diluted from an F18m glycerol stock of EC16m (1%) to a concentration of 4.7, 14, 47, 140, and 467 μM.

FIG. 7 is a graph showing the time response of antiviral activity of F18m (EC16m) nasal formulation. The formulation was diluted and then incubated with virus for 1, 5, 15, and 30 min.

FIGS. 8A and 8B are EM images of virus after 1-min incubation with diluted F18. The viruses appear deformed by binding to EC16.

FIG. 9 is a bar graph showing the time response of contact inhibition antiviral activity of F18D EC16 nasal formulation diluted with normal saline, to a concentration of 1.25 mM EC16. The antiviral activity was calculated and expressed as log₁₀ reduction with standard deviation. Results are from three independent experiments and the control is the formulation without EC16 incubated with virus for 30 min.

FIG. 10 is a bar graph showing MTT assay results. The primary cells were allowed to form a monolayer prior to incubation with F18m (orange bars) and normal saline (blue bars) for 60 min. The solutions were replaced with cell culture medium. The MTT assay was performed the next day using CytoSelect MTT Cell Proliferation Assay kit (Cell Biolabs, Inc).

FIG. 11 is a graph showing size distribution of particles in saline-diluted F18D. The size distribution profile for one representative sample determined by NTA is shown.

FIGS. 12A-12D are representative TEM images of Control (FIG. 12B, F18D formulation without EC16) and F18D EC16 nanoformulation treated OC43 virus (FIG. 12D). The Control treated virus showing intact structure with viral coat spike-like appearance (FIG. 12A). The F18D EC16 nanoformulation treated viral particles collapsed and disfigured (FIG. 12C).

FIGS. 13A-13F are representative TEM images of 30-minute exposure to Control (saline without EC16, FIG. 13C) and F18D EC16 nanoformulation of OC43 virus (FIG. 13F). The Control treated virus showing intact structure (FIG. 13A) with viral coat spike-like appearance (FIG. 13B). The F18D EC16 nanoformulation treated viral particles collapsed (FIG. 13E) with debris (FIG. 13D).

FIGS. 14A-14B are bar graphs showing the MTT cell viability assay results demonstrates that both formulations showed comparable cell viability with saline-treated cells for 60 minutes. When the F18BC (FIG. 14A) was diluted 2 or 5 times with basal medium, or F18DC (FIG. 14B) was diluted 5 times, the cell viability was higher than saline-treated cells.

FIGS. 15A-15F are EVOS images taken after 7-day post-infection of HNPEC by OC43 virus. The upper row are viral titer wells infected by 10⁻⁶, 10⁻⁷, 10⁻⁸ of virus without intervention. At 10⁻⁶ (FIG. 15A), 10⁻⁷ (FIG. 15C) concentrations, the cells were infected as died. At 10⁻⁸ dilution (FIG. 15E), the cells showed stress in morphology. In contrast, no cell infection was observed in the lower row (FIG. 15B, FIG. 15D, FIG. 15F), which was treated to 50 μM of EC16m in F18m formulation. This equals to a >2 log₁₀ reduction of viral replication in the cells.

FIGS. 16A-16B are graphs showing the cytotoxicity results of F100S (FIG. 16A) and F100SD (FIG. 16B) with HCT cells. These results show that EC16 in the F100 formulation did not have a cytotoxic effect on HCT cells, as measured by MTT, beyond the effect of the excipient formulation.

FIGS. 17A-17B are graphs showing the cytoxicity results of F100S (FIG. 17A) and F100SD (FIG. 17B) with MRC-5 cells. These results show that EC16 in the F100 formulation did not have a cytotoxic effect, as measured by MTT, beyond the modest effect of the excipient formulation.

FIG. 18 is a graph showing the effect of SHMP on F100 cytotoxicity with MRC-5 cells. When SHMP was included, the MTT values were not changed significantly. In the absence of SHMP, F18 was significantly higher than 66.3% saline and Saline+Glycerol, indicating a stimulation of cellular activity by F18 relative to the controls, but with no further increase due to SHMP.

FIG. 19 is a bar graph showing the result of normal saline/glycerol and F18 diluted in cell culture medium. Undiluted F18 has higher cell viability than undiluted saline, as well as medium diluted F18 1:1 (0.05% EC16m). Formulations diluted 1 to 5 and 1 to 10 have similar cell viability levels.

FIG. 20 is a bar graph showing the result of SHMP/saline/glycerol and F18D diluted in cell culture medium. Undiluted F18D has higher cell viability than undiluted SHMP/saline/glycerol. Formulations diluted 1 to 1, 1 to 5, and 1 to 10 have similar cell viability levels.

### DETAILED DESCRIPTION OF THE INVENTION

### I. Pharmaceutical Formulations

Described are pharmaceutical formulations for nasal administration. The pharmaceutical formulation contains a green tea catechin derivative; and optionally a carbohydrate; and a pharmaceutically acceptable carrier. In some forms, the pharmaceutical formulation contains a liquid pharmaceutically acceptable carrier and is in a liquid form, such as an emulsion or a suspension. The suspension can be an aqueous or minimally aqueous suspension. In some forms, the pharmaceutical formulation is a suspension containing nanoparticles of a green tea catechin derivative. In these forms, the pharmaceutically acceptable carrier(s) forming the suspension formulation can be/contain glycerol or a liquid poly(ethylene glycol), or a combination thereof; and optionally one or more of the following: an alcohol (e.g., methanol, ethanol, n-propanol, isopropanol, etc.), water, and an aqueous solution (e.g., normal saline, phosphate buffered saline, etc.). In some forms, the pharmaceutically acceptable carrier(s) forming the suspension formulation can be/contain glycerol; and optionally one or more of the following: an alcohol (e.g., methanol, ethanol, n-propanol, isopropanol, etc.), water, and an aqueous solution (e.g., normal saline, phosphate buffered saline, etc.).

The disclosed pharmaceutical formulations, in particular suspension formulations, are featured with high antiviral activity (i.e., at least 2 log₁₀ reduction of viral infectivity within 1 min), low cytotoxicity (e.g., as shown by an MTT value similar to (i.e., ±20%) a control treated with pharmaceutically acceptable carrier(s) only, tested using human nasal epithelial cells), and stability (e.g., no or minimal flocculation, precipitation, or creaming).

In some forms, the pharmaceutical formulation has a viscosity sufficient to maintain the green tea catechin derivative in contact with nasal epithelial cells for a therapeutically effective amount of time (and thus is also referred to as “mucoadhesive nasal formulation”). In some embodiments, the pharmaceutical formulation has a viscosity sufficient to maintain the green tea catechin derivative in contact with nasal epithelial cells for at least about 30 minutes in the presence of mucus. For example, the pharmaceutical formulation has a viscosity of at least about 15 cps, at least about 20 cps, at least about 25 cps, or at least about 30 cps and up to about 150 cps, such as in a range from about 15 cps to about 150 cps, from about 15 cps to about 125 cps, from about 15 cps to about 95 cps, from about 15 cps to about 75 cps, from about 15 cps to about 65 cps, from about 20 cps to about 65 cps, from about 25 cps to about 65 cps, from about 30 cps to about 65 cps, from about 35 cps to about 65 cps, from about 40 cps to about 65 cps, from about 45 cps to about 65 cps, from about 30 cps to about 60 cps, from about 35 cps to about 60 cps, from about 40 cps to about 60 cps, from about 35 cps to about 55 cps, from about 40 cps to about 55 cps, or from about 45 cps to about 55 cps, such as about 50 cps.

The disclosed nasal formulations are particularly useful for inhibiting or reducing viral replication in nasal epithelia and/or repairing viral-damaged nasal epithelial cells, neuroepithelial cells, vascular cells, and/or neurons, such as olfactory epithelium and central nervous system (CNS) tissues, optionally within minutes.

Nasal epithelia are the major initial site of entry for respiratory viruses, such as SARS-CoV-2, prior to spreading to upper respiratory tissues and invade into CNS. For example, multiciliated cells in the nasal respiratory epithelium serve as a reservoir for SARS-CoV-2 replication. Without being bound to any theories, it is believed that the disclosed pharmaceutical formulations can inhibit or reduce viral replication in nasal epithelia and/or neuroinvasion of CNS (e.g., inhibit or reduce viral replication by >99.99% within minutes), and thereby prevent or reduce neurological damage. It is also believed that the disclosed pharmaceutical formulations can promote cell differentiation, reduce inflammation/apoptosis, and/or reduce oxidative stress of nasal epithelial cells, neuroepithelial cells, vascular cells, and/or neurons caused by exposure to virus, and thereby repair the damaged nasal epithelial cells, neuroepithelial cells, vascular cells, and/or neurons and restore their functions.

For example, the human nasal cavity is made of the respiratory epithelium (RE), containing ciliated cells, basal cells, brush cells, and secretory cells, and the olfactory epithelium (OE). The olfactory epithelium contains olfactory sensory neurons, sustentacular cells, microvillar cells, globose basal cells, and horizontal basal cells. Entry of virus, such as a respiratory virus, e.g., SARS-CoV-2, into the nasal cavity results in infection and initial replication in the RE, during the early stage of viral infection, mainly in the ciliated cells. The rapid accumulation of virus in RE can cause concomitant infection in the OE, leading to OE destruction and anosmia (and other neuronal dysfunctions in the CNS). Persistent viral residence in the olfactory epithelium (months to years) is associated with olfactory dysfunction. Thus, active viral replication in RE, OE, and the olfactory bulb can be the cause of acute anosmia, and persistent presence of the virus in the RE and OE cells could be associated with chronic anosmia. It is believed that by promoting cell differentiation, reducing inflammation, and/or reducing oxidative stress of nasal epithelial cells caused by exposure to a virus such as SARS-CoV-2, the disclosed pharmaceutical formulations can restore the olfactory function of these cells.

Optionally, the disclosed pharmaceutical formulation further contains one or more pharmaceutically acceptable excipients, such as one or more of emulsifiers, dispersing agents, emollients, buffering agents, thickening agents, chelating agents, and preservatives; and/or one or more active agents (in addition to the green tea catechin derivative). In some forms, the disclosed pharmaceutical formulation does not contain a quaternary ammonium salt, such as benzalkonium chloride, cetylpyridimium chloride, benzethonium chloride, dioctadecyl dimethyl ammonium chloride, or octenidine dihydrochloride.

### A. Green Tea Catechin Derivatives

The pharmaceutical formulation disclosed herein contains a green tea catechin or derivative thereof, preferably a green tea catechin derivative and optionally in combination with a green tea catechin. In some embodiments, the pharmaceutical formulation disclosed herein contains a combination of two or more green tea catechin derivatives. Exemplary green tea catechins and derivatives thereof include, but are not limited to, epigallocatechin-3-gallate, epicatechin, epigallocatechin, and epicatechin-3-gallate, and their derivative thereof.

In preferred embodiments, the pharmaceutical formulation disclosed herein contains a green tea catechin derivative, such as a derivative of epigallocatechin-3-gallate, epicatechin, epigallocatechin, or epicatechin-3-gallate, or a combination thereof. The derivative of green tea catechin can be green tea catechins having chemical modifications to increase stability, lipid-solubility, and/or bioavailability in a host. For example, these chemical modifications to a green tea catechin include the addition of chemical groups having a charge under physiological conditions, and/or the conjugation of the green tea catechin to other biological moieties, such as polypeptides, carbohydrates, lipids, or a combination thereof.

Preferred modifications to the green tea catechin include modifications with one or more hydrocarbon chains having C1 to C30 groups. For example, the green tea catechin derivative of the pharmaceutical formulation contains a fatty/aromatic acyl group, such as a long chain aliphatic acyl group (i.e., having an aliphatic group with at least five carbon atoms). For example, the green tea catechin derivative of the pharmaceutical formulation contains one or more acyloxy groups, wherein the acyl group contains 1-30 carbon atoms, 5-30 carbon atoms, 8-30 carbon atoms, 10-30 carbon atoms, 8-25 carbon atoms, 10-25 carbon atoms, 13-21 carbon atoms, 14-25 carbon atoms, 14-20 carbon atoms, or 14-18 carbon atoms, such as 14, 15, or 16 carbon atoms.

It is believed that the addition of alkyl, alkenyl, or alkynyl chains/rings, aromatic rings, and/or lipids, such as via fatty acid esterification, to green tea catechins can increase the stability of the green tea catechins (e.g., prevent auto-oxidation of the catechin moieties) and/or the solubility of the green tea catechins in hydrophobic media including lipids, fats, soaps, detergents, surfactants, or oils, compared to unmodified green tea catechins. The increased stability and oil-solubility of the green tea catechin derivatives can improve the compounds' attachment to cell membranes at the delivery site (e.g., in contact with the target cells for a longer period of time) to perform antiviral, anti-inflammatory, and/or anti-oxidative actions, and thereby inhibit or reduce viral replication in nasal epithelia, and/or neuroinvasion of CNS, and/or repair damaged nasal epithelial cells, neuroepithelial cells, vascular cells, and/or neurons and restore their functions.

In some forms, the green tea catechin derivative or each green tea catechin derivative in the pharmaceutical formulation can have the structure of Formula I:

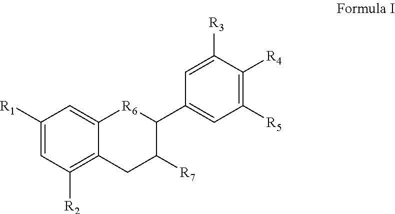

wherein: (i) R₁-R₅ and R₇ can be independently hydrogen, hydroxyl,

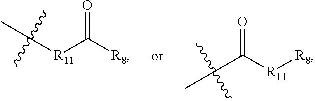

R₈ can be a substituted C₁-C₃₀ alkyl, an unsubstituted C₁-C₃₀ alkyl, a substituted C₂-C₃₀ alkenyl, an unsubstituted C₂-C₃₀ alkenyl, a substituted C₂-C₃₀ alkynyl, an unsubstituted C₂-C₃₀ alkynyl, a substituted aryl, an unsubstituted aryl, a substituted polyaryl, or an unsubstituted polyaryl; (ii) R₆ and R₁₁ can be independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; and (iii) at least one of R₁-R₅ and R₇ can be

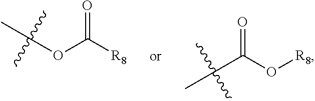

or a pharmaceutically acceptable salt thereof.

In some forms, the green tea catechin derivative or each green tea catechin derivative in the pharmaceutical formulation can have the structure of Formula II:

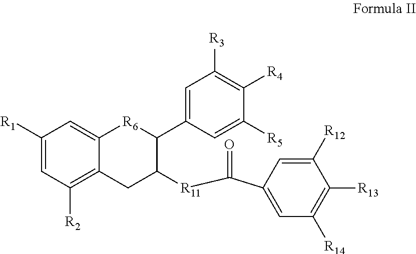

wherein: (i) R₁-R₅ and R₁₂-R₁₄ can be independently hydrogen, hydroxyl,

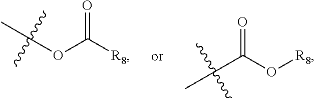

R₈ can be a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; (ii) R₆ and R₁₁ can be independently oxygen, —NR₉R₁₀, or sulfur, R₉ and R₁₀ can be independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, or an unsubstituted C₁-C₃₀ alkynyl; and (iii) least one of R₁-R₅ and R₁₂-R₁₄ can be

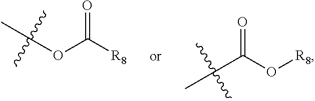

or a pharmaceutically acceptable salt thereof.

In some forms, the green tea catechin derivative can be esterified with at least two fatty acids, at least three fatty acids, or at least four fatty acids. For example, for Formula I, at least two, at least three, or at least four of R₁-R₅ and R₇ can be

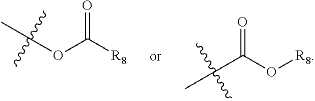

For Formula II, at least two, at least three, or at least four of R₁-R₅ and R₁₂-R₁₄ can be

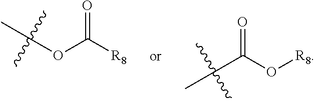

For any of Formulae I and II, R₆ and/or R₁₁ can be oxygen. For any of Formulae I and II, R₁ and R₂ can be hydroxyl. For any of Formulae I and II, at least one of R₃-R₅ can be

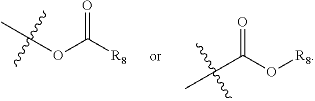

In some forms of Formula II, R₁₂-R₁₄ can be independently hydroxyl,

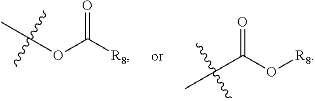

For any of Formulae I and II, R₈ can be a linear or branched substituted C₁-C₃₀ alkyl or a linear or branched unsubstituted C₁-C₃₀ alkyl, such as a linear or branched substituted C₁₄-C₂₅ alkyl or a linear or branched unsubstituted C₁₄-C₂₅ alkyl, for example, butanoic acid, hexanoic acid, octanoic acid, decanoic acid, dodecanoic acid, tetradecanoic acid, hexadecanoic acid (palmitic acid), 9-hexadecenoic acid, octadecanoic acid (stearic acid), 9-octadecenoic acid, 11-octadecenoic acid, 9,12-octadecadienoic acid, 9,12,15-octadecatrienoic acid, 6,9,12-octadecatrienoicacid, eicosanoic acid, 9-eicosenoic acid, 5,8,11,14-eicosatetraenoic acid, 5,8,11,14,17-eicosapentaenoic acid, docosanoic acid, 13-docosenoic acid, 4,7,10,13,16,19-docosahexaenoic acid, and tetracosanoic acid. In some forms, R₈ is a linear or branched C₁₅H₃₁.

For any of Formulae I and II, when one or more substituents are present, the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a carbonyl, an alkoxy, a halogen, a hydroxyl, a phenoxy, a thiol, an alkylthio, a phenylthio, an arylthio, a cyano, an isocyano, a nitro, a carboxyl, an amino, an amido, an oxo, a silyl, a sulfinyl, a sulfonyl, a sulfonic acid, a phosphonium, a phosphanyl, a phosphoryl, or a phosphonyl.

For any of Formulae I and II, when one or more substituents are present, the substituents can be independently a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted alkylaryl (e.g. benzyl), a carbonyl (e.g. carboxyl, ester, etc.), an alkoxy (e.g. methoxy, ethoxy, aryloxy, benzoether, etc.), a halide, a hydroxyl, or a haloalkyl, or a combination thereof.

For any of Formulae I and II, the alkyl can be a linear alkyl, a branched alkyl, or a cyclic alkyl (either monocyclic or polycyclic). The terms “cyclic alkyl” and “cycloalkyl” are used interchangeably herein. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkyl group.

For any of Formulae I and II, the alkenyl can be a linear alkenyl, a branched alkenyl, or a cyclic alkenyl (either monocyclic or polycyclic). The terms “cyclic alkenyl” and “cycloalkenyl” are used interchangeably herein. Exemplary alkenyl include a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃, C₂ alkenyl group, a branched C₃-C₉, C₃-C₉, C₃-C₈, C₃-C₇, C₃-C₆, C₃-C₅, C₃-C₄ alkenyl group, or a cyclic C₃-C₉, C₃-C₉, C₃-C₈, C₃-C₇, C₃-C₆, C₃-C₅, C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocylcic or polycyclic alkenyl group.

For any of Formulae I and II, the alkynyl can be a linear alkynyl, a branched alkynyl, or a cyclic alkynyl (either monocyclic or polycyclic). The terms “cyclic alkynyl” and “cycloalkynyl” are used interchangeably herein. Exemplary alkynyl include a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₂-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃, C₂ alkynyl group, a branched C₃-C₉, C₃-C₉, C₃-C₈, C₃-C₇, C₃-C₆, C₃-C₅, C₃-C₄ alkynyl group, or a cyclic C₃-C₉, C₃-C₉, C₃-C₈, C₃-C₇, C₃-C₆, C₃-C₅, C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkynyl group.

For any of Formulae I and II, the aryl group can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl. For any of Formulae I and II, the polyaryl group can be a C₁₀-C₃₀ polyaryl, a C₁₀-C₂₀ polyaryl, a C₁₀-C₁₂ polyaryl, a C₁₀-C₁₁ polyaryl, or a C₁₂-C₂₀ polyaryl. It is understood that the aryl can be a polyheteroaryl, such as a C₁₀-C₃₀ polyheteroaryl, a C₁₀-C₂₀ polyheteroaryl, a C₁₀-C₁₂ polyheteroaryl, a C₁₀-C₁₁ polyheteroaryl, or a C₁₂-C₂₀ polyheteroaryl.

See also U.S. Published Application No. 2020/0214290 and U.S. Published Application No. 2021/0196673, which are specifically incorporated by reference herein in its entirety.

### 1. Epigallocatechin-3-Gallate-Palmitate (“EGCG16” or “EC16”)

In some forms, the green tea catechin derivative of the pharmaceutical formulation is formed by the addition of acyl chains, for example via C₁-C₃₀ fatty acid esterification, to epigallocatechin-3-gallate (“EGCG”), which increases the stability and the solubility of EGCG in hydrophobic media. For example, the green tea catechin derivative of the pharmaceutical formulation contains EGCG esterified with palmitic acid or stearic acid at one or more of the 7, 5, 5′, 4′, 3′, 5″, 4″, and 3″ positions.

In some forms, the EGCG contained in the pharmaceutical formulation is esterified with palmitic acid at one (i.e., EGCG-mono-palmitate), two (i.e., EGCG-di-palmitate), or three (i.e., EGCG-tri-palmitate) of the 7, 5, 5′, 4′, 3′, 5″, 4″, and 3″ positions (collectively referred to herein as “epigallocatechin-3-gallate-palmitate,” “EGCG16,” or “EC16”). Thus, “EGCG16” or “EC16” typically refers to a mixture of EGCG-mono-palmitate, EGCG-di-palmitate, and EGCG-tri-palmitate, with the majority being EGCG-mono-palmitate, followed by EGCG-di-palmitates, and then EGCG-tri-palmitates. For example, the pharmaceutical formulation disclosed herein contains EGCG16, which is a mixture of EGCG-mono-palmitate, EGCG-di-palmitate, and EGCG-tri-palmitate, with EGCG-mono-palmitate being the majority. In these forms, the EGCG-mono-palmitate contained in the pharmaceutical formulation can be EGCG esterified with palmitic acid at the 4′ position (and thus is an EGCG-mono-palmitate, which is also referred to herein as “EC16m”), having the following structure.

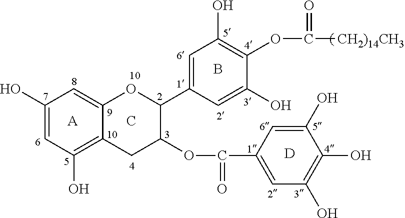

In preferred forms, the pharmaceutical formulation disclosed herein contains EGCG-mono-palmitate as the only form of ester, such as EC16m having the structure shown above.

The green tea catechin derivatives may be neutral or may be one or more pharmaceutically acceptable salts, crystalline forms, non-crystalline forms, hydrates, or solvates, or a combination thereof. References to the c green tea catechin derivatives may refer to the neutral molecule, and/or those additional forms thereof collectively and individually from the context.

Pharmaceutically acceptable salts of the compounds include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminum, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

### 2. Amount of Green Tea Catechin Derivatives

Typically, the green tea catechin derivative is present in the pharmaceutical formulation in an amount or concentration effective to inhibit or reduce viral replication in nasal epithelia and/or neuroinvasion of CNS, and/or repair damaged nasal epithelial cells, neuroepithelial cells, vascular cells, and/or neurons and restore their functions in a subject in need thereof.

For example, the green tea catechin derivative is present in the pharmaceutical formulation in an amount or concentration effective to kill ≥80%, ≥85%, ≥90%, ≥95%, ≥98%, ≥99%, ≥99.9%, ≥99.99%, ≥99.999%, or ≥99.9999% of virus, such as OC43, within 30 mins, within 20 mins, within 15 mins, within 10 mins, within 5 mins, within 2 mins, or within 1 min. For example, the green tea catechin derivative is present in the pharmaceutical formulation in an amount or concentration effective to reduce viral titer by at least 2 log₁₀, at least 3 log₁₀, at least 4 logo, at least 5 log₁₀, or at least 6 log₁₀, within 30 mins, within 20 mins, within 15 mins, within 10 mins, within 5 mins, within 2 mins, or within 1 min.

Additionally or alternatively, the green tea catechin derivative is present in the pharmaceutical formulation in an amount or concentration effective to reduce viral infectivity of nasal epithelia cells by ≥80%, ≥85%, ≥90%, ≥95%, ≥98%, ≥99%, ≥99.9%, or ≥99.9999% with ≤30 min, ≤20 min, ≤15 min, ≤10 min, ≤5 min, ≤2 min, or ≤1 min cell exposure to the pharmaceutical formulation before cell exposure to a virus. For example, the green tea catechin derivative is present in the pharmaceutical formulation in an amount or concentration effective to reduce viral infectivity by at least 2 log₁₀, at least 3 log₁₀, at least 4 log₁₀, at least 5 log₁₀, or at least 6 log₁₀, with ≤30 min, ≤20 min, ≤15 min, ≤10 min, ≤5 min, ≤2 min, or ≤1 min cell exposure to the pharmaceutical formulation before cell exposure to a virus. For example, the green tea catechin derivative (such as EC16 or EC16m) is present in the pharmaceutical formulation in an amount or concentration effective to reduce viral infectivity by at least 6 log₁₀, with ≤2 min or ≤1 min cell exposure to the pharmaceutical formulation before cell exposure to a virus.

In some forms, the effective amount of the green tea catechin derivative is from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation. For example, the pharmaceutical formulation contains EGCG-mono-palmitate (i.e., as the only form of ester), such as EC16m, in an effective amount (to inhibit or reduce viral replication in nasal epithelia and/or neutroinvation of CNS, and/or neurons and restore their functions in a subject thereof, such as those described above) from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation. For example, the pharmaceutical formulation contains EGCG16, where the total amount of EGCG16 (that is effective to inhibit or reduce viral replication in nasal epithelia and/or neutroinvation of CNS, and/or neurons and restore their functions in a subject thereof, such as those described above) is from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation. The term “total amount of EGCG16” refers to the total weight of EGCG16, which typically includes EGCG-mono-palmitate, EGCG-di-palmitate, and EGCG-tri-palmitate, in the pharmaceutical formulation divided by the total volume of the pharmaceutical formulation.

In some forms, the effective concentration of the green tea catechin derivative of the pharmaceutical formulation is from about 5 μM to about 5 mM, from about 10 μM to about 1.5 mM, from about 12.5 μM to about 1.25 mM, from about 12.5 μM to about 50 μM, or from about 50 μM to about 1.25 mM. For example, the pharmaceutical formulation contains EGCG-mono-palmitate (i.e., as the only form of ester), such as EC16m, at an effective concentration (to inhibit or reduce viral replication in nasal epithelia and/or neutroinvation of CNS, and/or neurons and restore their functions in a subject thereof, such as those described above) from about 5 μM to about 50 μM, from about 10 μM to about 50 μM, or from about 12.5 μM to about 50 μM. For example, the pharmaceutical formulation contains EGCG16, where the total concentration of EGCG16 (that is effective to inhibit or reduce viral replication in nasal epithelia and/or neutroinvation of CNS, and/or neurons and restore their functions in a subject thereof, such as those described above) is from about 10 μM to about 5 mM, from about 50 μM to about 1.5 mM, or from about 50 μM to about 1.25 mM. The term “total concentration of EGCG16” refers to the total mole of EGCG16, which typically includes EGCG-mono-palmitate, EGCG-di-palmitate, and EGCG-tri-palmitate, in the pharmaceutical formulation divided by the total volume of the pharmaceutical formulation.

When the pharmaceutical formulation disclosed herein contains a combination of two or more green tea catechin derivatives, such as two or more green tea catechin derivatives of Formula I or Formula II, each of the green tea catechin derivative can be in a suitable amount. For example, the pharmaceutical formulation disclosed herein contains three green tea catechin derivatives, wherein a first green tea catechin derivative can be in an amount of at least 50 wt %, at least 60 wt %, at least 70 wt %, at least 80 wt %, or at least 90 wt %; a second green tea catechin derivative can be in an amount of less than 40 wt %, less than 30 wt %, or less than 20 wt %; and a third green tea catechin derivative can be in an amount of less than 10 wt %, less than 5 wt %, or less than 2 wt % in the mixture.

For example, when the pharmaceutical formulation disclosed herein contains EGCG16, the EGCG-mono-palmitate (such as EC16m having the structure shown above) is in an amount of at least 50 wt %, at least 60 wt %, at least 70 wt %, at least 80 wt %, or at least 90 wt %; the EGCG-di-palmitate is in an amount of less than 40 wt %, less than 30 wt %, or less than 20 wt %, and the EGCG-tri-palmitate is in an amount of less than 10 wt %, less than 5 wt %, or less than 2 wt % in the mixture.

The precise amount of green tea catechin derivative in the pharmaceutical formulation can vary according to a variety of factors such as subject-dependent variables (e.g., age, immune system health, etc.), the virus infection or symptoms being treated, as well as the pharmacokinetics of the agents being administered.

### B. Carbohydrates

The pharmaceutical formulation disclosed herein may contain a carbohydrate, such as a sugar alcohol or a saccharide, or a combination thereof. In some forms, the carbohydrate of the pharmaceutical formulation is a sugar alcohol, such as glycerol. In some forms, the carbohydrate of the pharmaceutical formulation is a saccharide, such as a monosaccharide, disaccharide, oligosaccharide, or a polysaccharide, or a combination thereof. In some forms, the carbohydrate of the pharmaceutical formulation is a polysaccharide, such as a cellulose, a derivative thereof, or a salt thereof, or a combination thereof.

In some forms, the carbohydrate, such as a polysaccharide, of the pharmaceutical formulation can increase the solubility of the green tea catechin derivative and/or adjust the viscosity of the pharmaceutical formation to enhance the attachment of the green tea catechin derivate to cell membranes. For example, the disclosed pharmaceutical formulation contains a suitable polysaccharide, resulting in a viscosity of at least about 15 cps, at least about 20 cps, at least about 25 cps, or at least about 30 cps and up to about 65 cps, such as in a range from about 15 cps to about 150 cps, from about 15 cps to about 125 cps, from about 15 cps to about 150 cps, from about 15 cps to about 95 cps, from about 15 cps to about 75 cps, from about 15 cps to about 65 cps, from about 20 cps to about 65 cps, from about 25 cps to about 65 cps, from about 30 cps to about 65 cps, from about 35 cps to about 65 cps, from about 40 cps to about 65 cps, from about 45 cps to about 65 cps, from about 30 cps to about 60 cps, from about 35 cps to about 60 cps, from about 40 cps to about 60 cps, from about 35 cps to about 55 cps, from about 40 cps to about 55 cps, or from about 45 cps to about 55 cps, such as about 50 cps, for the pharmaceutical formulation. Such a viscosity of the pharmaceutical formulation allows the green tea catechin derivative to be in contact with nasal epithelial cells for a therapeutically effective amount of time, e.g., at least 30 minutes in the presence of mucus, following administration to the target site.

Generally, the amount of the carbohydrate is in a range from about 0.01% (w/v) to about 8% (w/v), from about 0.1% (w/v) to about 8% (w/v), from about 0.5% (w/v) to about 8% (w/v), from about 1% (w/v) to about 8% (w/v), from about 2% (w/v) to about 8% (w/v), from about 0.01% (w/v) to about 6% (w/v), from about 0.1% (w/v) to about 6% (w/v), from about 0.5% to about 6% (w/v), from about 1% to about 6% (w/v), from about 2% to about 6% (w/v), from about 0.01% (w/v) to about 4% (w/v), from about 0.1% (w/v) to about 4% (w/v), from about 0.5% to about 4% (w/v), from about 1% to about 4% (w/v), or from about 2% (w/v) to about 4% (w/v) of the pharmaceutical formulation, such as from about 0.01% (w/v) to about 0.2% (w/v).

### 1. Exemplary Polysaccharides

In some forms, the carbohydrate of the pharmaceutical formulation is a polysaccharide. Suitable polysaccharides for use in the disclosed pharmaceutical formulation include, but are not limited to, carboxymethylcellulose sodium, microcrystalline cellulose, xyloglucan, dendrobium officinale polysaccharide (DOP), hypomellose, glyceryl polymethacrylate, xanthan gum, hydroxyethylcellulose, guar gum, locust bean gum, carboxymethylcellulose, and hydroxypropylmethylcellulose.

For example, the polysaccharide of the pharmaceutical formulation is xyloglucan.

### 2. Amount of Polysaccharides

Typically, the polysaccharide is present in the pharmaceutical formulation in an amount effective to achieve a viscosity of at least 15 cPs and optionally up to about 65 cps for the nasal formulation, as determined by a viscometer.

For example, the amount of the polysaccharide is in a range from about 0.01% (w/v) to about 8% (w/v), from about 0.1% (w/v) to about 8% (w/v), from about 0.5% (w/v) to about 8% (w/v), from about 1% (w/v) to about 8% (w/v), from about 2% (w/v) to about 8% (w/v), from about 0.01% (w/v) to about 6% (w/v), from about 0.1% (w/v) to about 6% (w/v), from about 0.5% to about 6% (w/v), from about 1% to about 6% (w/v), from about 2% to about 6% (w/v), from about 0.01% (w/v) to about 4% (w/v), from about 0.1% (w/v) to about 4% (w/v), from about 0.5% to about 4% (w/v), from about 1% to about 4% (w/v), or from about 2% (w/v) to about 4% (w/v) of the pharmaceutical formulation.

The precise amount of polysaccharide in the pharmaceutical formulation can vary according to a variety of factors such as subject-dependent variables (e.g., age, immune system health, etc.), the virus infection or symptoms being treated, as well as the pharmacokinetics of the agents being administered.

### C. Pharmaceutically Acceptable Carriers

In some forms, the pharmaceutical formulation disclosed herein contains a pharmaceutically acceptable carrier, optionally more than one pharmaceutically acceptable carrier, depending on the form of the formulation.

For example, the disclosed pharmaceutical formulation contains at least one liquid pharmaceutically acceptable carrier and is in a liquid form, such as an emulsion or a suspension.

When the disclosed pharmaceutical formulation is an emulsion (also referred to herein as “emulsion formulation”), such as a nanoemulsion, it contains at least two pharmaceutically acceptable carriers. For example, the pharmaceutical formulation contains a first pharmaceutically acceptable carrier that is an aqueous solution or water (such as distilled water, deionized water, and/or tap water), a second pharmaceutically acceptable carrier that is an oil (such as an animal or vegetable oil), and optionally a third pharmaceutically acceptable carrier that is an organic solvent. Specific examples of aqueous solutions, oil, and organic solvents are described below.

When the disclosed pharmaceutical formulation is a suspension (also referred to herein as “suspension formulation”), it contains one or more pharmaceutically acceptable carriers. The pharmaceutically acceptable carrier or each pharmaceutically acceptable carrier in the suspension formulation can be any suitable solvent (organic or non-organic), as long as the solvent or the mixture of solvent (when more than one solvent is included) forms stable nanoparticles of a green tea catechin derivative. For example, the pharmaceutically acceptable carriers of a suspension formulation contain glycerol or a liquid poly(ethylene glycol), or a combination thereof; and optionally one or more of the following: an alcohol (e.g., methanol, ethanol, n-propanol, isopropanol, etc.), water, and an aqueous solution (e.g., normal saline, phosphate buffered saline, etc.). For example, the pharmaceutically acceptable carriers of a suspension formulation contain glycerol; and optionally one or more of the following: an alcohol (e.g., methanol, ethanol, n-propanol, isopropanol, etc.), water, and an aqueous solution (e.g., normal saline, phosphate buffered saline, etc.). When an alcohol is present in the suspension formulation, the alcohol is typically in a trace amount, i.e., ≤1 wt %.

The specific amount of the pharmaceutically acceptable carrier or each pharmaceutically acceptable carrier in the suspension formulation depends on the desired concentration of the green tea catechin derivative, the specific solvent, etc. For example, the suspension formulation contains a mixture of glycerol, an alcohol, and water/aqueous solution as pharmaceutically acceptable carriers, where the amount of glycerol range from 0.1 wt % to 99.5 wt %, the amount of the alcohol is ≤1 wt %, and the amount of water/aqueous solution range from 0.1 wt % to 99 wt %. More specific exemplary pharmaceutically acceptable carriers and the amount of each in exemplary suspension formulations are described in the Examples below.

The aqueous solution or water of the pharmaceutical formulation, either in an emulsion formulation or a suspension formulation, may be buffered or unbuffered. For example, the aqueous solution as a pharmaceutically acceptable carrier in the nasal formulation is water (such as distilled water, deionized water, and/or tap water) or a saline solution (normal, isotonic, or hypertonic). For example, the aqueous solution as a pharmaceutically acceptable carrier in the nasal formulation is a phosphate-buffered saline (PBS), a citrate buffer solution, EMEM medium (e.g., serum-free EMEM), MEM medium (e.g., serum-free MEM), and/or Hanks balanced salt solution, having a pH in a range from 3 to 7.4, from 4 to 7, from 4 to 6, from 4 to 5, from 5 to 7, or from 5 to 6, such as about 6. For example, the aqueous solution as a pharmaceutically acceptable carrier in the nasal formulation is a bicarbonate buffer. When the pharmaceutically acceptable carrier of the nasal formulation is water, one or more buffering agents can be dissolved in the water to maintain a constant pH in a range from 3 to 7.4, from 4 to 7, from 4 to 6, from 4 to 5, from 5 to 7, or from 5 to 6, such as about 6. When the pharmaceutically acceptable carrier of the nasal formulation is a saline solution (including normal, isotonic, or hypertonic saline), the pH of the formulation can be in a range from 3 to 8.5, from 4 to 8.2, or from 6 to 7.4. In some forms, the pH of the formulation containing saline solution as a pharmaceutically acceptable carrier has a near neutral pH, such as in a range from 5.6 to 7.5. In some forms, the aqueous solution or water of the pharmaceutical formulation may contain a small amount (such as less than 20 vol %, less than 10 vol %, less than 5 vol %, or less than 1 vol %) of a water-miscible organic solvent, such as ethanol or propanol, or a combination thereof.

The buffered solutions also affect the tonicity of the nasal formulations. For example, the total concentration of salts and/or buffering agents in the buffered solution is adjusted so that the nasal formulation is isotonic or hypertonic. The “total concentration of salts and/or buffering agents” refers to the total number of mole of the salts and/or buffering agents in the aqueous solution of the pharmaceutical formulation divided by the total volume of the aqueous solution of the pharmaceutical formulation. “Isotonic” refers to an osmotic pressure that is the same as the fluid present in the nasal membranes. “Hypertonic” refers to an osmotic pressure that is higher than the fluid present in the nasal membranes.

### D. Excipients

The pharmaceutical formulation disclosed herein may further contain one or more pharmaceutically acceptable excipients, such as one or more of emulsifiers, dispersing agents, emollients, buffering agents, thickening agents, chelating agents, and preservatives. In some forms, the disclosed pharmaceutical formulations, in particular suspension formulations, contain a dispersing agent, such as a carbohydrate or salt thereof (e.g., sodium carboxymethyl cellulose), or a metaphosphate (e.g., trimetaphosphate, hexametaphosphate, etc.) or salt thereof (e.g., sodium trimetaphosphate, sodium hexametaphosphate, etc.), or a combination thereof.

When a carbohydrate, such as any one of the polysaccharides described above, is included in the disclosed pharmaceutical formulation, it can increase the solubility of the green tea catechin derivative and/or adjust the viscosity of the pharmaceutical formation to enhance the attachment of the green tea catechin derivate to cell membranes, as described above, and may also act as a dispersing agent in the formulation. For example, in a suspension formulation, the carbohydrate, such as sodium carboxymethyl cellulose, can act as a dispersing agent to improve the separation of nanoparticles of the green tea catechin derivative and to prevent settling or clumping of the nanoparticles.

In some forms, the disclosed pharmaceutical formulations, in particular suspension formulations, contain a metaphosphate (such as trimetaphosphate, hexametaphosphate, etc.) or salt thereof (such as sodium trimetaphosphate, sodium hexametaphosphate, etc.). In some forms, the metaphosphate or salt thereof in the suspension formulations can further enhance the antiviral activity of the formulation. For example, the antiviral activity of a suspension formulation containing a metaphosphate (such as sodium hexametaphosphate) is higher than the antiviral activity of a suspension formulation containing the same components expect for the metaphosphate. For example, the viral titer reduction (log₁₀) of a suspension formulation containing a metaphosphate (such as sodium hexametaphosphate) is at least 1.1-time higher, at least 1.2-time higher, at least 1.5-time higher, at least 2-time higher, at least 3-time higher, at least 4-time higher, at least 5-time higher, or at least 10-time higher than the viral titer reduction (log₁₀) of a suspension formulation containing the same components expect for the metaphosphate, measured under the same conditions (e.g., same assay, same virus, same temperature, same pressure, same time period, etc.).

The total amount of the pharmaceutically acceptable excipients can be in a range from about 1% (w/v) to about 20% (w/v), from about 2% (w/v) to about 20% (w/v), from about 1% (w/v) to about 15% (w/v), from about 2% (w/v) to about 15% (w/v), from about 1% (w/v) to about 10% (w/v), from about 2% (w/v) to about 10% (w/v), from about 1% (w/v) to about 8% (w/v), from about 2% to about 8% (w/v), from about 0.5% to about 20% (w/v), or from about 0.5% (w/v) to about 15% (w/v) of the pharmaceutical formulation. The term “total amount of the pharmaceutically acceptable excipients” refers to the total weight of emulsifiers, emollients, buffering agents, thickening agents, chelating agents, and/or preservatives in the pharmaceutical formulation divided by the total volume of the pharmaceutical formulation. In some forms, the amount of each pharmaceutical acceptable excipient in the pharmaceutical formulation is up to 20% (w/v), up to 10% (w/v), or up to 5% (w/v), such as in a range from about 0.01% (w/v) to about 20% (w/v), from about 0.1% (w/v) to about 20% (w/v), from about 0.5% (w/v) to about 20% (w/v), from about 0.01% (w/v) to about 10% (w/v), from about 0.1% (w/v) to about 10% (w/v), from about 0.5% (w/v) to about 10% (w/v), from about 1% (w/v) to about 10% (w/v), from about 0.01% (w/v) to about 5% (w/v), from about 0.1% (w/v) to about 5% (w/v), from about 0.5% (w/v) to about 5% (w/v), from about 0.01% (w/v) to about 3% (w/v), from about 0.1% (w/v) to about 3% (w/v), from about 0.5% (w/v) to about 3% (w/v), from about 0.01% (w/v) to about 2% (w/v), from about 0.1% (w/v) to about 2% (w/v), or from about 0.5% (w/v) to about 2% (w/v).

### 1. Emulsifiers

Optionally, the disclosed pharmaceutical formulation contains one or more emulsifiers, in particular when the pharmaceutical formulation is in the form of emulsions, such as nanoemulsion. The emulsifier may also be included in a pharmaceutical acceptable carrier, such as water, saline (normal, isotonic, or hypertonic), or any one of the buffered solutions described above, when the pharmaceutical formulation is the form of aqueous suspensions. Any suitable emulsifiers can be used in the disclosed pharmaceutical formulations, such as non-ionic emulsifiers, anionic emulsifiers, cationic emulsifiers, or amphiphilic emulsifiers, or a combination thereof.

Exemplary emulsifiers suitable for use in the pharmaceutical formulation include, but are not limited to, metaphosphates (such as trimetaphosphate, hexametaphosphate, etc.) and salts thereof (such as sodium trimetaphosphate, sodium hexametaphosphate, etc.), poloxamers (such as poloxamer 407, poloxamer 101, poloxamer 105, poloxamer 108, poloxamer 122, poloxamer 123, poloxamer 124, poloxamer 181, poloxamer 182, poloxamer 183, poloxamer 184, poloxamer 185, poloxamer 188, poloxamer 212, poloxamer 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, poloxamer 407, poloxamer 105 benzoate, and poloxamer 182 dibenzoate), polysorbates (such as polysorbate 20, polysorbate 21, polysorbate 40, polysorbate 60, polysorbate 61, polysorbate 65, polysorbate 80, polysorbate 81, and polysorbate 85), Triton® X-100, nonoxynol-9, an ethoxylated surfactants (such as an alcohol ethoxylated, an alkyl phenol ethoxylated, a fatty acid ethoxylated, a monoalkaol amide ethoxylated, a sorbitan ester ethoxylated, a fatty amino ethoxylated, and an ethylene oxide-propylene oxide copolymer), bis(polyethylene glycol bis [imidazoyl carbonyl]), nonoxynol-9, bis(polyethylene glycol bis [imidazoyl carbonyl]), Brij® 35, Brij® 56, Brij® 72, Brij® 76, Brij® 92V, Brij® 97, Brij® 58P, Cremophor® EL, decaethylene glycol monododecyl ether, N-Decanoyl N-methylglucamine, n-Decyl alpha-D-glucopyranoside, decyl beta-D-maltopyranoside, n-Dodecanoyl-N methylglucamide, n-Dodecyl alpha-D-maltoside, n-dodecyl beta-D-maltoside, n-dodecyl beta-D-maltoside, heptaethylene glycol monodecyl ether, heptaethylene glycol monododecyl ether, heptaethylene glycol monotetradecyl ether, n-hexadecyl beta-D-maltoside, hexaethylene glycol monododecyl ether, hexaethylene glycol monohexadecyl ether, hexaethylene glycol monooctadecyl ether, hexaethylene glycol monotetradecyl ether, Igepal CA-630, Igepal CA-630, methyl-60-(N-heptylcarbamoyl)-alpha-D-glucopyranoside, nonaethylene glycol monododecyl ether, N—N nonanoyl-N-methylglucamine, octaethylene glycol monodecyl ether, octaethylene glycol monododecyl ether, octaethylene glycol monohexadecyl ether, octaethylene glycol monooctadecyl ether, octaethylene glycol monotetradecyl ether, octyl-beta-D-glucopyranoside, pentaethylene glycol monodecyl ether, pentaethylene glycol monododecyl ether, pentaethylene glycol monohexadecyl ether, pentaethylene glycol, polyethylene glycol ether, pentaethylene glycol monohexadecyl ether, pentaethylene glycol monohexyl ether, pentaethylene glycol monooctadecyl ether, pentaethylene glycol monooctyl ether, polyethylene glycol diglycidyl ether, polyethylene glycol ether W-1, polyoxyethylene 10 tridecyl ether, polyoxyethylene 100 stearate, polyoxyethylene 20 isohexadecyl ether, polyoxyethylene 20 oleyl ether, polyoxyethylene 40 stearate, polyoxyethylene 50 stearate, polyoxyethylene 8 stearate, polyoxyethylene bis(imidazolyl carbonyl), polyoxyethylene 25 propylene glycol stearate, saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Type 15-S-7, Type 15-S-9, Type NP-10, Type NP-4, Type NP-40, Type NP-7, Type NP-9, Type TMN-10, Type TMN-6, tetradecyl-beta-D-maltoside, tetraethylene glycol monodecyl ether, tetraethylene glycol monododecyl ether, tetraethylene glycol monotetradecyl ether, triethylene glycol monodecyl ether, triethylene glycol monododecyl ether, triethylene glycol monohexadecyl ether, triethylene glycol monooctyl ether, triethylene glycol monotetradecyl ether, Triton® CF-21, Triton® CF-32, Triton® DF-12, Triton® DF-16, Triton® GR-5M, Triton® QS-15, Triton® QS-44, Triton® X-100, Triton® X-102, Triton® X-15, Triton® X-151, Triton® X-200, Triton® X-207, Triton® X-114, Triton® X-165, Triton® X-305, Triton® X-405, Triton® X-45, Triton® X-705-70, tyloxapol, n-undecyl beta-D-glucopyranoside, semisynthetic cetylpyridimium chloride, benzalkonium chloride, benzethonium chloride, dioctadecyl dimethyl ammonium chloride, and octenidine dihydrochloride, and combinations thereof.

For example, the disclosed pharmaceutical formulation contains one or more emulsifiers, such as one or more of poloxamers, polysorbates (such as polysorbate 80), Triton® X-100, nonoxynol-9, cetylpyridimium chloride, benzalkonium chloride, benzethonium chloride, dioctadecyl dimethyl ammonium chloride, glycerin, propolene glycol, povidone, and octenidine dihydrochloride.

The amount of the emulsifiers in the pharmaceutical formulation can be in a range from about 0.01% (w/v) to about 5% (w/v), from about 0.1% (w/v) to about 5% (w/v), from about 0.5% (w/v) to about 5% (w/v), or from about 1% (w/v) to about 5% (w/v) of the pharmaceutical formulation.

### 2. Emollients

Optionally, the disclosed pharmaceutical formulation contains one or more emollients, to moisturize the mucous membrane and to prevent irritation. Any suitable emollients can be used in the disclosed pharmaceutical formulations, such as sorbitol, glycerin, eucalyptol, propolene glycol, polyethylene glycol, or macrogol glycerol fatty acid ester, or a mixture thereof. For example, the disclosed pharmaceutical formulation contains glycerin or propolene glycol, or a combination thereof.

The amount of the emollients in the pharmaceutical formulation can be in a range from about 1% (w/v) to about 20% (w/v), from about 1% (w/v) to about 15% (w/v), from about 1% (w/v) to about 10% (w/v), or from about 1% (w/v) to about 5% (w/v) of the pharmaceutical formulation.

### 3. Buffering Agents

Optionally, the disclosed pharmaceutical formulation contains one or more buffering agents, to maintain the pH of the pharmaceutical formulation at a value suitable for nasal administration, such as a pH in a range from 3 to 7.4, from 4 to 7, from 4 to 6, from 4 to 5, from 5 to 7, or from 5 to 6, such as about 6. When the pharmaceutical formulation contains a buffered aqueous solution as a pharmaceutically acceptable carrier, additional buffering agents are optional. When the pharmaceutical formulation contains water as a pharmaceutically acceptable carrier, one or more buffering agents are typically dissolved in the water to form a buffered solution.

Suitable buffering agents for use in the disclosed pharmaceutical formulation include, but are not limited to, diethyanolamine, lactic acid, monoethanolamine, triethylanolamine, citric acid, acetic acid, acetate, hydrogen phosphate, dihydrogen phosphate, carbonate, bicarbonate, borate, N-cyclohexyl-2-aminoethanesulfonic acid, sodium chloride, sodium hydroxide, sodium phosphate, semi-synthetic derivatives thereof, and combinations thereof.

For example, the disclosed pharmaceutical formulation contains citric acid, acetic acid, acetate, hydrogen phosphate, dihydrogen phosphate, carbonate, bicarbonate, borate, or N-cyclohexyl-2-aminoethanesulfonic acid, or a combination thereof.

The amount of the buffering agents in the pharmaceutical formulation depends on the particular pharmaceutically acceptable carrier(s) used in the formulation and the desired pH, and can be determined by a person skilled in the art.

### 4. Thickening Agents

Optionally, the disclosed pharmaceutical formulation contains one or more thickening agents, to further adjust the viscosity of the pharmaceutical formulation. Suitable thickening agents for use in the disclosed pharmaceutical formulation include, but are not limited to, carbomers, polyvinyl alcohol, povidone, colloidal silicon dioxide, cetyl alcohols, stearic acid, beeswax, petrolatum, triglycerides, phosphoglycerides, and lanolin, and combinations thereof.

The amount of the thickening agents in the pharmaceutical formulation depends on the particular pharmaceutically acceptable carrier(s) used in the formulation. Generally, the thickening agent(s) can be present in the pharmaceutical formulation in an amount from 0.5% (w/v) to about 10% (w/v), from about 0.5% (w/v) to about 5% (w/v), from about 1% (w/v) to about 10% (w/v), or from about 1% (w/v) to about 5% (w/v) of the pharmaceutical formulation.

### 5. Chelating Agents

Optionally, the disclosed pharmaceutical formulation contains one or more chelating agents. Suitable chelating agents for use in the disclosed pharmaceutical formulation include, but are not limited to, ethylenediamine, ethylenediaminetetraacetic acid (EDTA), ethylene glycol-bis(b-aminoethyl ether)-N,N,N′,N′-tetraacetic acid (EGTA), phytic acid, polyphosphoric acid, citric acid, gluconic acid, acetic acid, lactic acid, and dimercaprol, and combinations thereof. For example, the disclosed pharmaceutical formulation contains EDTA or EGTA, or a combination thereof.

The amount of the chelating agents in the pharmaceutical formulation depends on the particular pharmaceutically acceptable carrier(s) used in the formulation. Generally, the thickening agent(s) can be present in the pharmaceutical formulation in an amount from 0.0005% (w/v) to about 1% (w/v), from about 0.005% (w/v) to about 1% (w/v), from about 0.05% (w/v) to about 1% (w/v), or from about 0.005% (w/v) to about 0.5% (w/v) of the pharmaceutical formulation.

### 6. Preservatives

Optionally, the disclosed pharmaceutical formulation contains one or more preservatives, to maintain the shelf-stability of the pharmaceutical formulation, such as a shelf-life of at least 6 months, at least 1 year, at least 2 year, at least 3 years, or at least 5 years, at a temperature from 10 to 35° C. Suitable preservatives for use in the disclosed pharmaceutical formulation include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, benzyl alcohol, chlorhexidine (bis(p-chlorophenyldiguanido) hexane), chlorphenesin (3-(4-chloropheoxy)-propane-1,2-diol), Kathon CG (methyl and methyl chloroisothiazolinone), parabens (methyl, ethyl, propyl, butyl hydrobenzoates), phenoxyethanol (2-phenoxyethanol), sorbic acid (potassium sorbate, sorbic acid), phenonip (phenoxyethanol, methyl, ethyl, butyl, propyl parabens), phenoroc (phenoxyethanol 0.73%, methyl paraben 0.2%, propyl paraben 0.07%), Liquipar oil (isopropyl, isobutyl, butylparabens), Liquipar PE (70% phenoxyethanol, 30% liquipar oil), Nipaguard MPA (benzyl alcohol (70%), methyl & propyl parabens), Nipaguard MPS (propylene glycol, methyl, and propyl parabens), Nipasept (methyl, ethyl, and propyl parabens), Nipastat (methyl, butyl, ethyl, and propyel parabens), Elestab 388 (phenoxyethanol in propylene glycol plus chlorphenesin and methylparaben), and Killitol (7.5% chlorphenesin and 7.5% methyl parabens), and combinations thereof.

For example, the disclosed pharmaceutical formulation contains benzalkonium chloride or potassium sorbate, or a combination thereof. In some forms, the disclosed pharmaceutical formulation does not contain benzalkonium chloride, cetylpyridimium chloride, benzethonium chloride, dioctadecyl dimethyl ammonium chloride, or octenidine dihydrochloride.

The amount of the preservatives in the pharmaceutical formulation depends on the particular pharmaceutically acceptable carrier(s) used in the formulation. Generally, the preservatives can be present in the pharmaceutical formulation in an amount from 0.0005% (w/v) to about 1% (w/v), from about 0.005% (w/v) to about 1% (w/v), from about 0.05% (w/v) to about 1% (w/v), or from about 0.005% (w/v) to about 0.5% (w/v) of the pharmaceutical formulation.

### 7. Dispersing Agents

Optionally, the disclosed pharmaceutical formulation contains one or more dispersing agents. Suitable dispersing agents for use in the disclosed pharmaceutical formulation include, but are not limited to, polyvinyl pyrrolidone (PVP), sodium hexametaphosphate (SHP), sodium salt of EDTA (EDTA-Na), sodium dodecyl sulfonate (SDS), and sodium dodecyl benzene sulfonate (SDBS).

The amount of the dispersing agents in the pharmaceutical formulation depends on the particular pharmaceutically acceptable carrier(s) used in the formulation. Generally, the dispersing agent(s) can be present in the pharmaceutical formulation in an amount from 0.0005% (w/v) to about 1% (w/v), from about 0.005% (w/v) to about 1% (w/v), from about 0.05% (w/v) to about 1% (w/v), from about 0.005% (w/v) to about 0.5% (w/v), from about 0.01% (w/v) to about 5% (w/v), from about 0.1% (w/v) to about 5% (w/v), from about 0.5% (w/v) to about 5% (w/v), or from about 1% (w/v) to about 5% (w/v) of the pharmaceutical formulation.

### E. Additional Active Agents

The pharmaceutical formulation disclosed herein may contain, or be otherwise co-administered with, one or more active agents (in addition to the green tea catechin derivative), such as an anti-inflammatory agent, an antioxidant (such as vitamin E oil), or an antiviral agent, or a combination thereof. In some forms, the additional active agent is not a quaternary ammonium compound, such as benzalkonium chloride, cetylpyridimium chloride, benzethonium chloride, dioctadecyl dimethyl ammonium chloride, or octenidine dihydrochloride.

The total amount of the active agents in the pharmaceutical formulation can be in a range from about 0.01% (w/v) to about 10% (w/v); from about 0.01% (w/v) to about 1% (w/v); from about 0.01% (w/v) to about 0.75% (w/v); or from about 0.1% (w/v) to about 0.5% (w/v) of the pharmaceutical formulation. The term “total amount of the active agents” refers to the total weight of anti-inflammatory agents, antioxidants, and/or antiviral agents in the pharmaceutical formulation divided by the total volume of the pharmaceutical formulation.

### 1. Anti-Inflammatory Agents

Optionally, the disclosed pharmaceutical formulation contains one or more anti-inflammatory agents. Suitable anti-inflammatory agents for use in the disclosed pharmaceutical formulation include, but are not limited to, steroids, such as clobetasol, halobetasol, halcinonide, amcinonide, betamethasone, desoximetasone, diflucortolone, fluocinolone, fluocinonide, mometasone, clobetasone, desonide, hydrocortisone, prednicarbate, and triamcinolone, salts thereof, and combinations thereof; non-steroidal anti-inflammatory drugs, such as aceclofenac, aspirin, celecoxib, clonixin, dexibupáfen, dexketoprofen, diclofenac, diflunisal, droxicam, etodolac, etoricoxib, fenoprofen, flufenamic acid, flurbiprofen, ibuprofen, indomethacin, isoxicam, ketoprofen, ketorolac, licofelone, lornoxicam, loxoprofen, lumiracoxib, meclofenamic acid, mefenamic acid, meloxicam, nabumetone, naproxen, nimesulide, oxaprozin, parecoxib, phenylbutazone, piroxicam, rofecoxib, salsalate, sulindac, tenoxicam, tolfenamic acid, tolmetin, and valdecoxib, and combinations thereof; and combinations thereof.

### 2. Antioxidants

Optionally, the disclosed pharmaceutical formulation contains one or more antioxidants. Suitable antioxidants for use in the disclosed pharmaceutical formulation include, but are not limited to, vitamin C, vitamin E, beta-carotene, carotenoids, and minerals, such as selenium and manganese.

### 3. Antiviral Agents

Optionally, the disclosed pharmaceutical formulation contains one or more antiviral agents, such as those that can kill or inactivate respiratory virus such as a coronavirus, for example, SARS-CoV-2. Suitable antiviral agents for use in the disclosed pharmaceutical formulation include, but are not limited to, chloroquine, darunavir, galidesivir, interferon beta, lopinavir, ritonavir, remdesivir, and triazavirin, and combinations thereof.

### F. Forms of Formulation

Preferably, the pharmaceutical formulation disclosed herein is in a liquid form suitable for nasal administration, such as emulsion (for example, a nanoemulsion) or aqueous suspension. Although not illustrated herein, it is understood that the pharmaceutical formulation can omit the pharmaceutically acceptable carrier and be provided in a dry powder form for nasal administration.

### 1. Emulsion

In some forms, the disclosed pharmaceutical formulation is an emulsion, such as a nanoemulsion.

In these forms, at least two pharmaceutically acceptable carriers are used in the pharmaceutical formulation to form at least two phases, i.e., one or more aqueous phase and one or more oil phase. For example, the pharmaceutical formulation contains two pharmaceutically acceptable carriers, where a first pharmaceutically acceptable carrier is an aqueous solution or water (such as distilled water, deionized water, and/or tap water) that forms an aqueous phase, and where a second pharmaceutically acceptable carrier is an oil that forms an oil phase.

Optionally, the pharmaceutical formulation further contains a third pharmaceutically acceptable carrier that is an organic solvent, such as DMSO, an alcohol (for example, ethanol), glycerol, or an essential oil. The organic solvent can be miscible with water/aqueous solution or oil in the pharmaceutical formulation, and thus can increase the solubility of the green tea catechin derivative and/or one or more of the pharmaceutically acceptable excipients in the aqueous or oil phase, depending on the property of the organic solvent selected.

The two or more pharmaceutically acceptable carriers can form a water in oil emulsion, an oil in water emulsion, a water in oil in water emulsion, or an oil in water in oil emulsion.

In some forms, the emulsion is an oil in water emulsion, where the oil forms oil droplets in the aqueous solution or water, i.e., the oil phase dispersed in the aqueous phase is in a form of plurality of oil droplets. In these forms, the green tea catechin derivative is encapsulated in the oil droplets.

a. Oil Phase

An oil phase, in the form of a plurality of droplets, such as a plurality of nanodroplets, may be formed from any suitable lipid compound, such as an oil compound or a membrane lipid (e.g., a phosphoglyceride, for example, phosphatidylcholine). The lipid compounds forming the oil phase in the form of droplets, including oil compounds and membrane lipids, are referred to as “oil.” Typically, the droplets have an average diameter of less than 1 micron, less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 150 nm, less than or equal to about 100 nm, or less than or equal to about 50 nm, such as in a range from about 100 nm to about 600 nm, from about 150 nm to about 600 nm, from about 200 nm to about 600 nm, from about 250 nm to about 600 nm, from about 300 nm to about 600 nm, from about 300 nm to about 500 nm, or from about 300 nm to about 400 nm.

The oil forming the emulsion may be a membrane lipid (such as a phosphoglyceride, e.g., phosphatidylcholine) or any pharmaceutically acceptable oil suitable for nasal administration, such as animal oils, plant oils, vegetable oils, natural oils, synthetic oils, hydrocarbon oils, silicone oils, and combinations thereof. Examples of oils suitable for forming the oil in water emulsion include, but are not limited to, mineral oil, squalene oil, flavor oils, silicon oil, essential oils, water insoluble vitamins, isopropyl stearate, butyl stearate, octyl palmitate, cetyl palmitate, tridecyl behenate, diisopropyl adipate, dioctyl sebacate, menthyl anthranhilate, cetyloctanoate, octyl salicylate, isopropyl myristate, neopentyl glycol dicarpate cetols, Ceraphyls®, decyl oleate, diisopropyl adipate, C12-15 alkyl lactates, cetyl lactate, lauryl lactate, isostearyl neopentanoate, myristyl lactate, isocetyl stearoyl stearate, octyldodecyl stearoyl stearate, hydrocarbon oils, isoparaffin, fluid paraffins, isododecane, petrolatum, argan oil, canola oil, chile oil, coconut oil, corn oil, cottonseed oil, flaxseed oil, grape seed oil, mustard oil, olive oil, palm oil, palm kernel oil, peanut oil, pine seed oil, poppy seed oil, pumpkin seed oil, rice bran oil, safflower oil, tea oil, truffle oil, vegetable oil, apricot (kernel) oil, jojoba oil (Simmondsia chinensis seed oil), grapeseed oil, macadamia oil, wheat germ oil, almond oil, rapeseed oil, gourd oil, soybean oil, sesame oil, hazelnut oil, maize oil, sunflower oil, hemp oil, bois oil, kuki nut oil, avocado oil, walnut oil, fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, winter green leaf oil, flower oil, chamomile oil, clary sage oil, clove oil, geranium flower oil, hyssop flower oil, jasmine flower oil, lavender flower oil, manuka flower oil, marhoram flower oil, orange flower oil, rose flower oil, ylang-ylang flower oil, bark oil, cassia bark oil, cinnamon bark oil, sassafras bark oil, wood oil, camphor wood oil, cedar wood oil, rosewood oil, sandalwood oil, rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, oleic acid, linoleic acid, oleyl alcohol, and isostearyl alcohol, and combinations thereof.

For example, the oil forming the emulsion is soybean oil, avocado oil, squalene oil, olive oil, canola oil, corn oil, rapeseed oil, safflower oil, sunflower oil, fish oils, cinnamon bark, coconut oil, cottonseed oil, flaxseed oil, pine needle oil, silicon oil, mineral oil, essential oil, flavor oils, or water insoluble vitamins, or a combination thereof. For example, the oil forming the emulsion is a membrane lipid, such as a phosphoglyceride, for example, phosphatidylcholine.

The amount of oil in the emulsion depends on the aqueous phase, the amount of green tea catechin derivative, the type and amount of any additional active agents, and the properties of the particular oil forming oil droplets. Generally, the oil is present in an amount up to about 10 vol % or up to about 5 vol %, such as in a range from about 0.1 vol % to about 10 vol %, from about 0.5 vol % to about 10 vol %, from about 1 vol % to about 10 vol %, from about 0.1 vol % to about 5 vol %, from about 0.5 vol % to about 5 vol %, from about 1 vol % to about 5 vol %, from about 0.1 vol % to about 4 vol %, from about 0.5 vol % to about 4 vol %, from about 1 vol % to about 4 vol %, or from about 0.1 vol % to about 2 vol % of the pharmaceutical formulation. The amount of oil is determined by dividing the volume of oil used to form the oil droplet by the volume of oil, water/aqueous solution, and optionally organic solvents.

b. Aqueous Phase

The aqueous phase is formed by water or an aqueous solution in which the oil is able to disperse in the form of oil droplets. The water forming the aqueous phase may be purified water, filtered water, deionized water, distilled water, double distilled water, or water containing mineral impurities. The aqueous solution is typically a buffered aqueous solution, formed by dissolving one or more salts and/or buffering agents in water. For example, the aqueous solution of the emulsion is a phosphate-buffered saline (PBS) solution, a citrate buffer solution, MEM medium, normal saline, or Hanks balanced salt solution. Typically, the buffered aqueous solution has a pH in a range from 3 to 7.4, from 4 to 7, from 4 to 6, from 4 to 5, from 5 to 7, or from 5 to 6, such as about 6.

In some forms, the water used alone or for forming the aqueous solution of the emulsion is deionized water. In some forms, the water used alone or for forming the aqueous solution of the emulsion is purified water. In some forms, the water used alone or for forming the aqueous solution of the emulsion is sterile and/or pyrogen free.

The amount of water/aqueous solution in the emulsion depends on the oil phase, the desired amount of green tea catechin derivative, and the type and amount of any additional active agents. Generally, the water/aqueous solution is present in an amount of at least 85 vol %, such as in a range from about 85 vol % to about 99 vol %, from about 85 vol % to about 95 vol %, from about 85 vol % to about 90 vol %, from about 90 vol % to about 99 vol %, or from about 90 vol % to about 95 vol % of the pharmaceutical formulation. The amount of water/aqueous solution is determined by dividing the volume of water used to form the aqueous phase by the total volume of water/aqueous solution, oil, and optionally organic solvents.

c. Organic Solvents

The emulsions described herein can optionally include one or more organic solvents. The organic solvent can be miscible with water/aqueous solution or oil of the emulsion, and thus can increase the solubility of the green tea catechin derivative and/or one or more of the pharmaceutically acceptable excipients in the aqueous phase or oil phase, depending on the property of the organic solvent selected.

Exemplary organic solvents suitable for use in the emulsions include, but are not limited to, C1-C12 alcohols, diols, triols, such as ethanol, methanol, isopropyl alcohol, and glycerol; organic phosphate solvents, such as dialkyl phosphates and trialkyl phosphates having one to ten carbon atoms; medium chain triglycerides, diethyl ether, ethyl acetate, acetone, dimethyl sulfoxide (DMSO), acetic acid, n-butanol, butylene glycol, perfumers alcohol, isopropanol, n-propanol, formic acid, propylene glycol, glycerol, sorbitol, industrial methylated spirit, triacetin, hexane, benzene, toluene, diethyl ether, chloroform, 1,4-dioxane, tetrahydrofuran, dichloromethane, acetone, acetonitrile, dimethylformamide, dimethyl sulfoxide, and formic acid, and combinations thereof. The alkyl groups of the di- or tri-alkyl phosphate can all the same or the alkyl groups can be different, for example, tri-n-butyl phosphate.

When one or more organic solvents are included in the emulsion, the amount of the organic solvent in the emulsion depends on the property of the particular organic solvent used, the desired amount of green tea catechin derivative, the type and amount of any additional active agents, and the type and amount of any pharmaceutically acceptable excipients. Generally, the total amount of the one or more organic solvents is in a range from about 0.1 vol % to about 5 vol %, from about 0.1 vol % to about 1 vol %, or from about 1 vol % to about 5 vol % of the pharmaceutical formulation. The total amount of organic solvent(s) is determined by dividing the volume of organic solvent(s) in the emulsion by the total volume of water/aqueous solution, oil, and organic solvents.

d. Emulsifiers

Typically, when the pharmaceutical formulation is an emulsion, one or more emulsifiers are included in the emulsion to facilitate the formation of oil droplets and stabilize the oil droplets dispersed in the aqueous phase.

Any one of the above-described emulsifiers can be used for forming the emulsion. For example, the pharmaceutical formulation contains at least one emulsifier, such as a non-ionic emulsifier (e.g., poloxamer, polysorbate, Triton® X-100, and/or nonoxynol-9) and/or a cationic emulsifier (e.g., cetylpyridimium chloride, benzalkonium chloride, benzethonium chloride, dioctadecyl dimethyl ammonium chloride, or octenidine dihydrochloride, or a combination thereof). In some forms, the emulsifier is polysorbate 80. In some forms, the emulsifier is not a quaternary ammonium compound, such as cetylpyridimium chloride, benzalkonium chloride, or benzethonium chloride.

### 2. Suspensions

In some forms, the disclosed pharmaceutical formulation is an aqueous or a minimally aqueous suspension. In some forms, the suspension formulations are featured with high antiviral activity (i.e., at least 2 log 10 reduction of viral infectivity within 1 min), low cytotoxicity (e.g., as shown by an MTT value similar to a control treated with pharmaceutically acceptable carrier(s) only, tested using human nasal epithelial cells), and stability (e.g., no or minimal flocculation, precipitation, or creaming). Suspension formulations of green tea catechin derivative having a combination of these features were previously unobtainable. Without being bound to any theories, it may be that the green tea catechin derivative particles are formed from liquid crystals of the green tea catechin derivative.

The suspension formulation contains nanoparticles of a green tea catechin derivative dispersed in one or more suitable liquid pharmaceutically acceptable carrier(s), and optionally one or more pharmaceutically acceptable excipients, such as a dispersing agent (e.g., any one of those described above, such as a metaphosphate). Typically, the amount of the green tea catechin derivative in the suspension formulation is from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v). Such an amount of green tea catechin derivative in the suspension formulation can be effective to inhibit or reduce viral replication in nasal epithelia and/or neuroinvasion of the CNS, and/or repair damaged nasal epithelial cells, neuroepithelial cells, vascular cells, and/or neurons and restore their functions in a subject in need thereof.

The pharmaceutically acceptable carrier or each pharmaceutically acceptable carrier in the suspension formulation can be any suitable solvent (organic or non-organic), as long as the solvent or the mixture of solvent (when more than one solvent is included) forms stable nanoparticles of a green tea catechin derivative. In some forms, a suspension formulation contains glycerol or a liquid poly(ethylene glycol), or a combination thereof; and optionally one or more of the following: an alcohol (e.g., methanol, ethanol, n-propanol, isopropanol, etc.), water, and an aqueous solution (e.g., normal saline, phosphate buffered saline, etc.), as the pharmaceutically acceptable carriers. In some forms, a suspension formulation contains glycerol or a liquid poly(ethylene glycol), or a combination thereof; water or an aqueous solution, such as any one of those described above; and optionally an alcohol, as the pharmaceutically acceptable carriers. In some forms, a suspension formulation contains glycerol; and optionally one or more of the following: an alcohol (e.g., methanol, ethanol, n-propanol, isopropanol, etc.), water, and an aqueous solution (e.g., normal saline, phosphate buffered saline, etc.), as the pharmaceutically acceptable carriers. In some forms, a suspension formulation contains glycerol; water or an aqueous solution, such as any one of those described above; and optionally an alcohol, as the pharmaceutically acceptable carriers. When an alcohol is included in the suspension formulation as a pharmaceutically acceptable carrier, it is typically present at a trace amount, i.e., ≤1 wt %, such as from about 0.1 wt % to about 0.3 wt % of an alcohol.

For example, the suspension formulation contains glycerol, water, and a trace amount of alcohol (i.e., ≤1 wt %, such as from about 0.1 wt % to about 0.3 wt %). For example, the suspension formulation contains glycerol, a trace amount of alcohol (i.e., ≤1 wt %, such as from about 0.1 wt % to about 0.3 wt %), and an aqueous solution such as any one of those described above (e.g., saline solution (normal, isotonic, or hypertonic), phosphate buffered saline, a citrate buffer solution, MEM medium, normal saline, or Hanks balanced salt solution).

The different pharmaceutically acceptable carriers in the suspension formulation can have any suitable ratio. The specific amount of each pharmaceutically acceptable carrier in the suspension formulation depends on the desired concentration of the green tea catechin derivative, the specific solvent, etc.

For example, when the suspension formulation contains a mixture of glycerol, an alcohol, and water/aqueous solution as pharmaceutically acceptable carriers, the amount of glycerol can range from 0.1 wt % to 99.5 wt %, the amount of the alcohol is ≤1 wt %, and the amount of water/aqueous solution can range from 0.1 wt % to 99 wt %. In some forms, the amount of glycerol can range from 0.1 wt % to 35 wt %, and the amount of water/aqueous solution can range from 60 wt % to 99 wt % in the suspension formulation. In some forms, the amount of glycerol can range from 10 wt % to 99.5 wt % or from 40 wt % to 99.5 wt %, and the amount of water/aqueous solution can range from 0.1 wt % to 30 wt % or 0.1 wt % to 15 wt % in the suspension formulation. The ratio of glycerol:water/aqueous solution can vary from 10:1 to 1:100 or 1:1 to 1:100, such as 1:1, 1:5, 1:10, 1:20, 1:50, or 1:100. More specific exemplary pharmaceutically acceptable carriers and the amount of each in exemplary suspension formulations are described in the Examples below.

When the pharmaceutically acceptable carrier(s) of the suspension formulation is or contains an aqueous buffer or buffering agent, the formulation can have a pH in a range from 3 to 8.5, from 3 to 7.4, from 4 to 7, from 4 to 6, from 4 to 5, from 5 to 7, or from 5 to 6, such as about 6. When the pharmaceutically acceptable carrier(s) of the nasal formulation is or contains a saline solution (such as a normal saline solution), the pH of the formulation can be in a range from 3 to 8.5, from 4 to 8.2, or from 6 to 7.4. In some forms, the pH of the formulation containing saline solution as a pharmaceutically acceptable carrier has a near neutral pH, such as in a range from 5.6 to 7.5.

The pharmaceutically formulation in the form of a suspension may further include one or more of the pharmaceutical acceptable excipients described above, such as one or more emulsifiers, dispersing agents, emollients, buffering agents, thickening agents, chelating agents, and/or preservatives. In preferred forms, the suspension formulation includes a dispersing agent, such as a carbohydrate or salt thereof (e.g., sodium carboxymethyl cellulose), or a metaphosphate (e.g., trimetaphosphate, hexametaphosphate, etc.) or salt thereof (e.g., sodium trimetaphosphate, sodium hexametaphosphate, etc.), or a combination thereof. When a carbohydrate is included in the suspension formulation, it can increase the solubility of the green tea catechin derivative and/or adjust the viscosity of the formulation to enhance the attachment of the green tea catechin derivate to cell membranes, as described above, and may also act as a dispersing agent in the formulation. For example, in a suspension formulation, the carbohydrate, such as sodium carboxymethyl cellulose, can act as a dispersing agent to improve the separation of nanoparticles of the green tea catechin derivative and to prevent settling or clumping of the nanoparticles. In some forms, the suspension formulations contain a metaphosphate (such as trimetaphosphate, hexametaphosphate, etc.) or salt thereof (such as sodium trimetaphosphate, sodium hexametaphosphate, etc.).

The dispersing agent, such as a metaphosphate, can be present in an amount as described above, such as from 0.0005% (w/v) to about 1% (w/v), from about 0.005% (w/v) to about 1% (w/v), from about 0.05% (w/v) to about 1% (w/v), from about 0.005% (w/v) to about 0.5% (w/v), from about 0.01% (w/v) to about 0.5% (w/v), from about 0.1% (w/v) to about 0.5% (w/v), from about 0.005% (w/v) to about 2% (w/v), from about 0.01% (w/v) to about 2% (w/v), or from about 0.1% (w/v) to about 2% (w/v) of the suspension formulation.

In some forms, the metaphosphate or salt thereof in the suspension formulations can further enhance the antiviral activity of the formulation. For example, the antiviral activity of a suspension formulation containing a metaphosphate (such as sodium hexametaphosphate) is higher than the antiviral activity of a suspension formulation containing the same components expect for the metaphosphate. For example, the viral titer reduction (log₁₀) of a suspension formulation containing a metaphosphate (such as sodium hexametaphosphate) is at least 1.1-times higher, at least 1.2-times higher, at least 1.5-times higher, at least 2-times higher, at least 3-times higher, at least 4-times higher, at least 5-times higher, or at least 10-times higher than the viral titer reduction (log₁₀) of a suspension formulation containing the same components expect for the metaphosphate, measured under the same conditions (e.g., same assay, same virus, same temperature, same pressure, same time period, etc.).

The pharmaceutically formulation in the form of an aqueous suspension may also include one or more additional active agents as described above, such as one or more anti-inflammatory agents, antioxidants, and/or antiviral agents. When additional active agent(s) is present in the pharmaceutical formulation, the additional active agent is preferably not a quaternary ammonium compound, such as benzalkonium chloride, cetylpyridimium chloride, benzethonium chloride, dioctadecyl dimethyl ammonium chloride, or octenidine dihydrochloride.

The green tea catechin derivative is in the form of nanoparticles suspended in the liquid pharmaceutically acceptable carrier, such as a mixture of glycerol and water/aqueous solution, and optionally a trace amount of alcohol. These green tea catechin derivative particles typically have an average diameter of <5 μm, <4 μm, <3.5 μm, <3 μm, <2.5 μm, <2 μm, or <1 μm. In some forms, the green tea catechin derivative particles in the aqueous suspension can have a fine particle fraction of >40%, >45%, >50%, >55%, or >60%. The term “fine particle fraction” refers to the weight percentage of particles with a diameter less than 5 μm relative to the total weight of particles in the pharmaceutical formulation.

In some forms, the green tea catechin derivative particles suspended in the water or aqueous solution have a size distribution in a range from about 10 nm to about 1100 nm, from about 20 nm to about 1000 nm, or from about 40 nm to about 1000 nm, with a majority distributed in a range from about 100 nm to about 1000 nm. In these forms, the green tea catechin derivative particles can have an average diameter ranging from about 20 nm to about 1 μm, from about 50 nm to about 500 nm, from about 20 nm to about 500 nm, from about 20 nm to about 250 nm, from about 50 to about 250 nm, from about 100 nm to about 500 nm, from about 100 nm to about 550 nm, from about 100 nm to about 750 nm, or from about 20 nm to about 100 nm. Further, in these forms, the green tea catechin derivative particles in the aqueous suspension can have a fine particle fraction of >90%, >95%, or >99%, where at least >50%, >55%, >60%, >65%, >70%, >75%, >80%, or >90% particles have a diameter less than 1 μm. Further, in these forms, the green tea catechin derivative particles in the aqueous suspension can have a density of at least about 1×10⁷/ml, at least about 2×10⁷/ml, at least about 2.5×10⁷/ml, or at least about 3×10⁷/ml.

In preferred forms, the green tea catechin derivative particles are nanoparticles having a median diameter of less than 500 nm, less than 400 nm, less than 300 nm, or less than 200 nm, such as ranging from about 10 nm to about 500 nm, from about 10 nm to about 400 nm, from about 10 nm to about 300 nm, from about 10 nm to about 200 nm, from about 10 nm to about 150 nm, from about 50 nm to about 500 nm, from about 50 nm to about 400 nm, from about 50 nm to about 300 nm, from about 50 nm to about 200 nm, or from about 50 nm to about 150 nm. The mean diameter of the green tea catechin derivative can be determined using known methods, for example, by using nanoparticle tracking analysis (NTA). In these forms, the suspension formulation can be also referred to as a nanoformulation.

The green tea catechin derivative particles generally have a negative zeta potential, such as in a range from about-80 mV to about-10 mV or from about-50 mV to about-20 mV. Methods for measuring the zeta potential of the green tea catechin nanoparticles are known, such as by using a commercial zeta potential analyzer.

In some forms, the suspension formulation disclosed herein can be produced using the following method: (i) dissolving a green tea catechin derivative in a first pharmaceutically acceptable carrier to form a green tea catechin derivative solution; and (ii) mixing the green tea catechin derivative solution with a second pharmaceutically acceptable carrier to produce a suspension formulation. Optionally, the methods further include (iii) mixing the suspension formulation with a third pharmaceutically acceptable carrier. The optional step (iii) can be used to adjust the concentration of the green tea catechin derivative, the ratio of the different pharmaceutically acceptable carriers, and/or properties (e.g., viscosity) of the suspension formulation for a specific use. Each pharmaceutically acceptable carrier used in the method for producing the suspension formulation can be any one of those described above. For example, the first pharmaceutically acceptable carrier is a solvent that can dissolve the green tea catechin derivative, such as an alcohol (e.g., methanol, ethanol, n-propanol, isopropanol, etc.), or a mixture of water and an alcohol; the second pharmaceutically acceptable carrier is glycerol or a water mixture thereof; and the third pharmaceutically acceptable carrier is water or an aqueous solution (e.g., saline (normal, isotonic, or hypertonic), phosphate buffered saline, culture medium, etc.). In some forms, one or more of the pharmaceutically acceptable carriers used in the disclosed method contain(s) one or more pharmaceutically acceptable excipients described above, such as one or more emulsifiers, dispersing agents, emollients, buffering agents, thickening agents, chelating agents, and/or preservatives. For example, the third pharmaceutically acceptable carrier, such as water or an aqueous solution, contains one or more emulsifiers, dispersing agents, emollients, buffering agents, thickening agents, chelating agents, and/or preservatives, such as a dispersing agent, for example, a metaphosphate or salt thereof.

More specific exemplary pharmaceutically acceptable carriers that can be used for producing the suspension formulations are described in the Examples below.

### G. Viscosity

In some forms, the pharmaceutical formulation disclosed herein has a viscosity sufficient to maintain the green tea catechin derivative in contact with nasal epithelial cells for a therapeutically effective amount of time, e.g., at least about 30 minutes in the presence of mucus, following administration, and optionally without causing a stuffy nose. For example, the pharmaceutical formulation has a viscosity of at least about 15 cps, at least about 20 cps, at least about 25 cps, or at least about 30 cps and up to about 65 cps, such as in a range from about 15 cps to about 150 cps, from about 15 cps to about 125 cps, from about 15 cps to about 95 cps, from about 15 cps to about 75 cps, from about 15 cps to about 65 cps, from about 20 cps to about 65 cps, from about 25 cps to about 65 cps, from about 30 cps to about 65 cps, from about 35 cps to about 65 cps, from about 40 cps to about 65 cps, from about 45 cps to about 65 cps, from about 30 cps to about 60 cps, from about 35 cps to about 60 cps, from about 40 cps to about 60 cps, from about 35 cps to about 55 cps, from about 40 cps to about 55 cps, or from about 45 cps to about 55 cps, such as about 50 cps, as determined by a viscometer.

### H. Exemplary Formulations

Exemplary nasal formulations are described herein.

An exemplary nasal formulation contains one or more of the green tea catechin derivatives described herein; a surfactant, such as polysorbate 80 in an amount from about 0.3% (w/v) to about 1.5% (w/v). Optionally, a small amount of benzalkonium chloride (i.e., 0.01% (w/v) to 0.02% (w/v)) may be included as a preservative.

An exemplary method for forming an oil-in-water emulsion described herein includes adding an oil containing lipid (such as jojoba oil or vitamin E oil containing phosphatidylcholine, the oil is 0.5 vol % to 5 vol %) in water or an aqueous solution. The lipids can facilitate the formation of the oil-in-water emulsion. Such an oil-in-water emulsion can be further processed into a nanoemulsion by sonication and/or homogenization at a suitable strength for a suitable time period (such as sonication for 30 to 150 seconds). The size of the oil droplets can be measured by a ZetaView system. The viscosity can be adjusted using a carbohydrate, to a value under about 100 cps.

More specific exemplary formulations are described in the Examples below.

### II. Delivery Devices And Kits

Delivery devices and kits including one or more of the pharmaceutical formulations are also disclosed.

### A. Delivery Device

The pharmaceutical formulation disclosed herein can be delivered in any suitable container. Suitable containers for use in the delivery device can provide one or more single dosage or multi-dosages of the pharmaceutical formulation contained therein for the desired application. In preferred forms, the pharmaceutical formulation is in a liquid form, such as an emulsion or aqueous suspension, that can be delivered in a suitable container. Such suitable container can deliver the emulsion or aqueous suspension intranasally or via inhalation, such as dropper and spray bottles and any suitable unpressurized spray device or pressurized spray device, such as an inhaler or nebulizer.

Nasal delivery devices, such as sprays, nose droppers or needle-less syringes, may be employed to target the agent to different regions of the nasal cavity. OptiMist™ is a breath actuated device that targets liquid or powder nasal formulations to the nasal cavity, including the olfactory region, without deposition in the lungs or esophagus (Djupesland et al., Laryngoscope, 116:466 (2006)). The ViaNase™ device can also be used to target a nasal spray to the olfactory and respiratory epithelia of the nasal cavity. Nasal drops tend to deposit on the nasal floor and are subjected to rapid mucociliary clearance, while nasal sprays are distributed to the middle meatus of the nasal mucosa (Scheibe et al., Arch. Otolaryngol. Head Neck Surg., 134:643 (2008)). See also Djupesland, Drug Delivery and Translational Research volume 3, pages 42-62 (2013), which is specifically incorporated by reference herein in its entirety.

An exemplary delivery device includes a pressurized metered dose inhaler, having the pharmaceutical formulation contained therein. Such a metered dose inhaler can deliver a unit dosage of the pharmaceutical formulation per puff, which can be adjusted based on subject-dependent variables (e.g., age, immune system health, etc.), the disease or disorder being treated, as well as the pharmacokinetics of the agent being administered. For example, each puff of the pressurized metered dose inhaler is configured to deliver a unit dosage of the pharmaceutical formulation, which contains the green tea catechin derivative in an amount ranging from about 0.01 mg to about 20 mg, from about 0.01 mg to about 10 mg, from about 0.05 mg to about 20 mg, from about 0.05 mg to about 10 mg, from about 0.1 mg to about 20 mg, from about 0.1 mg to about 10 mg, from about 0.1 mg to about 5 mg, from about 0.5 mg to about 20 mg, from about 0.5 mg to about 10 mg, from about 0.5 mg to about 5 mg, from about 1 mg to about 20 mg, or from about 1 mg to about 10 mg.

### B. Kit

The pharmaceutical formulation disclosed herein may be included in a kit, along with a suitable application tool for applying the pharmaceutical formulation intranasally by an end user. The application tool may be packaged together or separately with the pharmaceutical formulation in the kit.

An exemplary kit includes one or more nasal swabs and the pharmaceutical formulation, such as an emulsion or aqueous suspension. The one or more nasal swabs are typically sterile. The nasal swab(s) and the pharmaceutical formulation may be packaged separately. For example, the pharmaceutical formulation is packaged in a suitable container and the nasal swab(s) are packaged in a pouch. Alternatively, the nasal swab may be impregnated or saturated with the pharmaceutical formulation. The impregnated/saturated nasal swab is packaged in a sealed pouch prior to use.

### III. Methods of Using the Pharmaceutical Formulations

Methods of using the pharmaceutical formulations disclosed herein including those with and without a carbohydrate and/or pharmaceutically acceptable carrier, are described. For example, the disclosed pharmaceutical formulations are used for preventing or reducing neurological damage caused by exposure to a virus, and/or for repairing nasal epithelial cells, neuroepithelial cells, vascular cells, and/or neurons damaged by a virus, in a subject in need thereof. In some embodiments, the compositions and methods reduce viral titer and/or migration of the virus in the subject, maintain or restore homeostasis and/or cell differentiation, reduce inflammation, and/or reduce oxidative stress to nasal epithelial cells, neuroepithelial cells, vascular cells, and/or neurons caused by a virus.

It will be appreciated the disclosed methods can be methods of treatment of the symptoms and conditions described herein. “Treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Thus, for example, methods of therapeutic and prophylactic methods of treating long COVID and symptoms thereof including, but not limited to, anosmia, are expressly provided.

The virus can be a respiratory virus, preferably a coronavirus, such as a SARS-CoV-2. Other exemplary respiratory viruses include, but are not limited to, respiratory syncytial virus, parainfluenza virus, adenovirus, rhinovirus, and other coronaviruses.

Generally, the methods can include (i) administering the disclosed pharmaceutical formulation to the nasal vestibule or passages of the subject. The administration step may be repeated one or more times for a period of time. In some forms, the administration step is repeated once, twice, or three times, per day, for a period of one day, three days, one week, two weeks, one month, three months, six months, or one year. For example, the administration step is performed twice a day for a period of one day, three days, one week, two weeks, one month, three months, six months, or one year.

In some forms, the disclosed methods is for reducing viral infection in a subject in need thereof. The subject may have not been exposed to the virus, have been exposed to the virus, or is infected with the virus. The virus is typically a respiratory virus, such as influenza virus (e.g., influenza A, influenza virus B, or influenza virus C), respiratory syncytial virus (RSV), human metapneumovirus, parainfluenza virus, adenovirus, rhinovirus, or a coronaviruses (e.g., a betacoronavirus, such as Human Coronavirus OC43 (HCoV-OC43), a Severe acute respiratory syndrome-related virus, optionally, SARS-CoV BtKY72, SARS-CoV-2, SARSr-CoV RaTG13, SARS-CoV PC4-227, or SARS-CoV, such as one that infects humans such as SARS-CoV or SARS-CoV-2, or a Middle East respiratory syndrome-related virus such as MERS-CoV), or a combination thereof.

Any of the methods can include administering an effective amount of a disclosed formulation and/or contacting the target nasal epithelial cells and/or neuroepithelial cells effective amount of time. An effective amount or therapeutically effective amount means a dosage and/or other element (e.g., amount of time) sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered.

For example, any of the methods can administer an effective amount or concentration of the disclosed pharmaceutical formulation to the nasal vestibule or passages of the subject (i.e., post-viral infection) to kill ≥80%, ≥85%, ≥90%, ≥95%, 98%, 99%, or ≥99.9%, such as ≥99%, ≥99.9%, ≥99.99%, ≥99.999%, ≥99.9999%, of a respiratory virus within 30 mins, within 20 mins, within 15 mins, within 10 mins, within 5 mins, within 2 mins, or within 1 min. For example, any of the methods can administer an effective amount or concentration of the disclosed pharmaceutical formulation to the nasal vestibule or passages of the subject (i.e., post-viral infection) to reduce viral titer by at least 2 log₁₀, at least 3 log₁₀, at least 4 log₁₀, at least 5 log₁₀, or at least 6 log₁₀, within 30 mins, within 20 mins, within 15 mins, within 10 mins, within 5 mins, within 2 mins, or within 1 min.

Additionally or alternatively, any of the method can administer an effective amount of the disclosed pharmaceutical formulation to the nasal vestibule or passages of the subject (i.e., pre-viral infection) to reduce viral infectivity of the target nasal epithelial cells and/or neuroepithelial cells by >80%, ≥85%, ≥90%, ≥95%, ≥98%, ≥99%, or ≥99.9%, such as ≥99%, ≥99.9%, ≥99.99%, ≥99.999%, ≥99.9999%, with ≤30 min, ≤20 min, ≤15 min, ≤10 min, ≤5 min, ≤2 min, or ≤1 min cell exposure to the pharmaceutical formulation before cell exposure to a virus. In some forms, any of the methods can administer an effective amount of the disclosed pharmaceutical formulation to the nasal vestibule or passages of the subject (i.e., pre-viral infection) to reduce viral infectivity of the target nasal epithelial cells and/or neuroepithelial cells by at least 2 log₁₀, at least 3 log₁₀, at least 4 log₁₀, at least 5 log₁₀, or at least 6 log₁₀, with ≤30 min, ≤20 min, ≤15 min, ≤10 min, ≤5 min, ≤2 min, or ≤1 min target cell exposure to the pharmaceutical formulation before cell exposure to a virus. For example, any of the methods can administer an effective amount of a disclosed pharmaceutical formulation containing EC16 or EC16m to the nasal vestibule or passages of the subject (i.e., pre-viral infection) to reduce viral infectivity of the target nasal epithelial cells and/or neuroepithelial cells by at least 6 log₁₀, with ≤2 min or ≤1 min target cell exposure to the pharmaceutical formulation before cell exposure to a virus.

The pharmaceutical formulation may be administered to the nasal vestibule or passages using any suitable method, such as by flushing or rinsing using a wash bottle, spraying using a spray bottle or a pressurized spray device, such as an inhaler, or swapping using a nasal swap. In some forms, the pharmaceutical formulation is provided in a pressured spray device, such as a pressurized metered dose inhaler, and is administrated by a puff into the nasal vestibule or passage. In some forms, the pharmaceutical formulation is administrated using a nasal swap that is saturated with the liquid formulation. Typically, following the administration or each administration, a unit dosage is delivered to the nasal epithelial cells of the subject, such as by each puff of the pressurized metered dose inhaler or each swap of a nasal swap pre-saturated with a determined amount of the pharmaceutical formulation. For example, each puff of the pressurized metered dose inhaler delivers a unit dosage of the pharmaceutical formulation, which contains an amount of the green tea catechin derivative (such as EC16) ranging from about 0.01 mg to about 20 mg, from about 0.01 mg to about 10 mg, from about 0.05 mg to about 20 mg, from about 0.05 mg to about 10 mg, from about 0.1 mg to about 20 mg, from about 0.1 mg to about 10 mg, from about 0.1 mg to about 5 mg, from about 0.5 mg to about 20 mg, from about 0.5 mg to about 10 mg, from about 0.5 mg to about 5 mg, from about 1 mg to about 20 mg, or from about 1 mg to about 10 mg.

In some forms, delivery to the CNS along neural pathways can be associated with delivery of the composition to the upper third of the nasal cavity (Hanson et al., BMC Neurosci., 9:S5 (2008)). Although a supine position may be employed another position for targeting the olfactory region is with the “praying to Mecca” position, with the head down-and-forward. A supine position with the head angle at 70° or 90° may also be suitable for efficient delivery to the CSF using a tube inserted into the nostrils to deliver the drug via intranasal administration (van den Berg et al., J. Neurosci. Methods, 116:99 (2002)).

In some forms of intranasal drug administration, nose drops may be administered over a period of time, e.g., 10-20 minutes to alternating nostrils every e.g., 1-2 minutes to allow the solution to be absorbed into the nasal epithelium (Thorne et al., Neuroscience, 127:481 (2004); Capsoni et al., Proc. Natl. Acad. Sci. USA, 99:12432 (2002); Ross et al., J. Neuroimmunol., 151:66 (2004); Ross et al., Neurosci. Lett., 439:30 (2008); Dhuria et al., J. Pharmacol. Exp. Ther., 328:312 (2009a); Dhuria et al., J. Pharm. Sci., 98:2501 (2009b); Francis et al., Brain, 131:3311 (2008); Martinez et al., Neuroscience, 157:908 (2008)). This noninvasive method does not involve inserting the device into the nostril, instead, drops are placed at the opening of the nostril, allowing the individual to sniff the drop into the nasal cavity. Other administration methods in anesthetized individual involve sealing the esophagus and inserting a breathing tube into the trachea to prevent the nasal formulation from being swallowed and to eliminate issues related to respiratory distress (Chow et al., J. Pharm. Sci., 88:754 (1999); Chow et al., J. Pharm. Sci., 90:1729 (2001); Fliedner et al., Endocrinology., 17:2088 (2006); Dahlin et al., Eur. J. Pharm. Sci., 14:75 (2001)). Flexible tubing can be inserted into the nostrils for localized delivery of a small volume of the drug solution to the respiratory or olfactory epithelia, depending on the length of the tubing (Chow et al., J. Pharm. Sci., 88:754 (1999); Van den Berg et al., J. Drug Target, 11:325 (2003); van den Berg et al., Pharm. Res., 21:799 (2004a); Banks et al., J. Pharmacol. Exp. Ther., 309:469 (2004); van den Berg et al., J. Neurosci. Methods, 116:99 (2002); Vyas et al., J. Pharm. Sci., 95:570 (2006a); Charlton et al., J. Drug Target, 15:370 (2007a); Gao et al., Int. J. Pharm., 340:207 (2007a)).

The formulation and delivery device can be selected and prepared to drive absorption through the nasal mucosa. The nasal mucosa is, compared to other mucous membranes, easily accessible and provides a practical entrance portal for small and large molecules (Bitter, et al., “Nasal Drug Delivery in Humans,” in Surber, et al., (eds): Topical Applications and the Mucosa. Curr Probl Dermatol. Basel, Karger, 2011, vol 40, pp 20-35, Pires, et al., J Pharm Pharmaceut Sci., 12(3) 288-311, 2009, and Djupesland, Drug Deliv, and Transl. Res., 3:42-62 (2013) DOI 10.1007/s13346-012-0108-9). Intranasal administration offers a rapid onset of therapeutic effects, reduced first-pass effect, reduced gastrointestinal degradation and lung toxicity, noninvasiveness, essentially painless application, and easy and ready use by patients—particularly suited for children—or by physicians in emergency settings. Flu Mist®, for example, is an exemplary effective nasal influenza vaccine spray.

Numerous delivery devices are available for intranasal administration. Devices vary in accuracy of delivery, dose reproducibility, cost and ease of use. Metered-dose systems provide dose accuracy and reproducibility. Differences also exist in force of delivery, spray patterns and emitted droplet size. The latter being important for drug deposition within the nasal cavity. Parameters can be can be modulated to enhance deposition while limiting the fraction of small particles able to bypass the nose and enter the lungs, or reduce deposition while increasing the fraction of small particles able to bypass the nose and enter the lungs.

The following aspects of nasal anatomy can influence drug delivery. During exhalation the soft palate closes automatically, separating the nasal and oral cavities. Thus, it becomes possible to use smaller particles in a nasal spray and still avoid lung deposition. Additionally, during closure of the soft palate there is a communication pathway between the two nostrils, located behind the walls separating the two passages. Under these circumstances, it is possible for airflow to enter via one nostril and leave by the other. This bidirectional delivery concept combines the two anatomical facts into one fully functional device. The device is inserted into one nostril by a sealing nozzle, and the patient blows into the mouthpiece. The combination of closed soft palate and sealed nozzle creates an airflow which enters one nostril, turns 180° through the communication pathway and exits through the other nostril (bidirectional flow). Since delivery occurs during exhalation, small particles cannot enter the lungs.

Particle size, flow rate and direction can be tuned for efficient delivery to the nasal mucosa. By adding an exit resistor to give additional control of the input pressure, it is possible to improve distribution to the sinuses and the middle ear. Manipulation of the flow pattern enables delivery to the olfactory region, thereby possibly achieving direct “nose-to-brain” delivery. The 180-degree turn behind the septum will trap particles still airborne, allowing targeted delivery of cargo to the adenoid.

Strategies for enhancing drug absorption via the nasal route are also known in the art and can be utilized in the disclosed formulations and methods of delivery. Such strategies include, for example, use of absorption enhancers such as surfactants, cyclodextrins, protease inhibitors, and tight junction modulators, as well as application of carriers such as liposomes and nanoparticles. See, e.g., Ghadiri, et al., Pharmaceutics, 11(3): 113 (2019). The method may further includes administering an additional active agent, such as an anti-inflammatory agent, an antioxidant, or antiviral agent, or a combination thereof to the subject, prior to, during, and/or after step (i) administration of the pharmaceutical formulation. Any active agents described above may be used in the method. The active agents may be included in the pharmaceutical formulation and thus administered together with the green tea catechin derivative. Alternatively, the active agents are administered separately, such as by oral administration, nasal administration, and/or intravenous administration.

The subject can be a mammal, such as human. The subject can be one who was exposed, is exposed to, and/or is expected to be exposed to a virus. For example, the subject was in close contact with an individual infected by the virus, as indicated by having a positive test result of the virus or having one or more symptoms of the viral infection, such as fever, cough, shortness of breath, diarrhea, sneezing, runny nose, and/or sore throat. The term “in close contact” can be, for example, being 6 feet of less away from an infected person for a total of 15 minutes or more over a 24-hour period, providing care at home to someone who is infected, having direct physical contact with the person (hugged or kissed them) who is infected, sharing eating or drinking utensils with an infected person, having been sneezed on, coughed on, or otherwise by contacted by respiratory droplets from an infected person.

In some embodiments, the subject has long COVID. In some embodiments, the subject has anosmia.

In some embodiments, the subject was identified in contact-tracing as having been exposed to the virus, or one or more subjects infected therewith.

In some embodiments, the subject will be exposed to the virus. An exemplary subject is a healthcare worker that will treat infected people.

The administration step can be performed before and/or after the subject is exposed to the virus.

In some forms, the administration step is performed after the subject is exposed to and infected by the virus. In some embodiments, treatment begins 1, 2, 3, 4, 5, or more hours, days, or weeks after exposure to the virus. In these forms, the pharmaceutical formulation can be administrated within 24 hours, within 18 hours, within 12 hours, within 6 hours, within 4 hours, within 2 hours, or within 1 hour after the subject is tested positive for a viral infection (counting from the time point that positive test result is received by the subject) or after the subject shows one or more symptoms of a viral infection, such as one or more of fever, cough, shortness of breath, diarrhea, sneezing, runny nose, and/or sore throat. Typically, the methods administer an effective amount or concentration of the disclosed pharmaceutical formulation to the nasal vestibule or passages of the subject (i.e., post-viral infection) to kill ≥80%, ≥85%, ≥90%, ≥95%, ≥98%, ≥99%, or ≥99.9%, such as ≥99%, ≥99.9%, ≥99.99%, ≥99.999%, ≥99.9999%, of a respiratory virus within 30 mins, within 20 mins, within 15 mins, within 10 mins, within 5 mins, within 2 mins, or within 1 min. In some forms, the methods administer an effective amount or concentration of the disclosed pharmaceutical formulation to the nasal vestibule or passages of the subject (i.e., post-viral infection) to reduce viral titer by at least 2 log₁₀, at least 3 log₁₀, at least 4 log₁₀, at least 5 log₁₀, or at least 6 log₁₀, within 30 mins, within 20 mins, within 15 mins, within 10 mins, within 5 mins, within 2 mins, or within 1 min.

In some forms, the administration step is performed before the subject is infected by the virus. In some embodiments, treatment begins 1, 2, 3, 4, 5, or more hours, days, or weeks prior to exposure to the virus. In these forms, the pharmaceutical formulation can be administrated within 24 hours, within 18 hours, within 12 hours, within 6 hours, within 4 hours, within 2 hours, or within 1 hour before the subject is exposed or expected to be exposed to the virus. In some forms, the pharmaceutical formulation can be administrated when the subject is exposed to the virus. In these forms, the methods administer an effective amount of the disclosed pharmaceutical formulation to the nasal vestibule or passages of the subject (i.e., pre-viral infection) to reduce viral infectivity of the target nasal epithelial cells and/or neuroepithelial cells by ≥80%, ≥85%, ≥90%, ≥95%, ≥98%, ≥99%, or ≥99.9%, such as ≥99%, ≥99.9%, ≥99.99%, ≥99.999%, ≥99.9999%, with ≤30 min, ≤20 min, ≤15 min, ≤10 min, ≤5 min, ≤2 min, or ≤1 min target cell exposure to the pharmaceutical formulation. In some forms, the methods administer an effective amount of the disclosed pharmaceutical formulation to the nasal vestibule or passages of the subject (i.e., pre-viral infection) to reduce viral infectivity of the target nasal epithelial cells and/or neuroepithelial cells by at least 2 log₁₀, at least 3 log₁₀, at least 4 log₁₀, at least 5 log₁₀, or at least 6 log₁₀, with ≤30 min, ≤20 min, ≤15 min, ≤10 min, ≤5 min, ≤2 min, or ≤1 min target cell exposure to the pharmaceutical formulation. For example, the methods administer an effective amount of a disclosed pharmaceutical formulation containing EC16 or EC16m to the nasal vestibule or passages of the subject (i.e., pre-viral infection) to reduce viral infectivity of the target nasal epithelial cells and/or neuroepithelial cells by at least 6 log₁₀, with ≤2 min or ≤1 min target cell exposure to the pharmaceutical formulation.

### A. Preventing or Reducing Neurological Damage

The pharmaceutical formulations disclosed herein can be used for preventing or reducing neurological damage caused by exposure to a virus, for example a respiratory virus such as a SARS-CoV-2 including but limited to variants of the original isolate (e.g., B.1.617.2 (Delta) and/or B.1.1.529 (Omicron)), in a subject in need thereof.

Without being bound to any theories, it is believed that the disclosed pharmaceutical formulations can inhibit or reduce viral replication in nasal epithelia and/or neuroinvasion of CNS, and thereby prevent or reduce neurological damage caused by the virus. For example, by preventing or reducing neurological damage caused by a virus such as a SARS-CoV-2, symptoms of long COVID, including headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, is minimized in subjects exposed to the virus.

Typically, following the administration or all of the administrations of the disclosed pharmaceutical formulation, an effective amount of the green tea catechin derivative is administered to the subject to prevent or reduce neurological damage caused by exposure to the virus, such as a SARS-CoV-2. For example, following the administration or all of the administrations of the disclosed pharmaceutical formulation, the subject (while still tested positive for the virus or after recovered from the infection as indicated by a negative positive for the virus) shows no symptom or less symptoms associated with neurological damages caused by exposure to the virus, such as headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, or gastrointestinal issues, or a combination thereof, compared to a control. The control is a subject administered with a nasal formulation without the green tea catechin derivative, such as a normal saline nasal formulation.

In some forms, the administration step is performed before an expected exposure of the subject to the virus. In these forms, administration of the pharmaceutical formulation may protect the subject from getting infected by the virus after exposure. Alternatively, the subject may become asymptomatically infected (i.e., having a positive test result for the virus without any symptoms) or show minor symptoms of a viral infection. For example, the subject may be infected by the virus, as indicated by a positive test result for the virus, while showing no symptom or less symptoms associated with neurological damages caused by the virus, such as headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, or gastrointestinal issues, or a combination thereof, compared to a control.

B. Repairing Damaged Nasal Epithelial, Neuroepithelial, and/or Vascular Cells, and/or Neurons

The pharmaceutical formulations disclosed herein can be used for repairing nasal epithelial cells, neuroepithelial cells, vascular cells, and/or neurons damaged by a virus, for example a respiratory virus such as a SARS-CoV-2 (including e.g., B.1.617.2 (Delta) and/or B.1.1.529 (Omicron)), in a subject in need thereof. Without being bound to any theories, it is believed that the disclosed pharmaceutical formulations can promote cell differentiation, reduce inflammation, and/or reduce oxidative stress of nasal epithelial cells, neuroepithelial cells, vascular cells, and/or neurons caused by a virus, and thereby repair the damaged nasal epithelial cells, neuroepithelial cells, vascular cells, and/or neurons and restore their functions.

Generally, the method includes (i) administering a disclosed pharmaceutical formulation to the nasal vestibule or passages of the subject. The administration can be administered while, or after the subject, is recovering/recovered from a viral infection. In some embodiments, the subject has a negative test result post-infection, but shows one or more symptoms associated with neurological damages caused by the virus.

The administration step may be repeated one or more times for a period of time, as described above. For example, the administration step is performed twice a day for a period of one day, three days, one week, two weeks, one month, three months, six months, or one year.

The pharmaceutical formulation may be administered to the nasal vestibule or passages using any suitable method, such as those described above.

The method may further includes administering an additional active agent to the subject, prior to, during, and/or after step (i) administration of the pharmaceutical formulation, as described above.

The subject is typically a mammal who was infected by a virus and recovered, as indicated by a negative test result for the virus. Although recovered from the viral infection, the subject shows one or more symptoms associated with neurological damage caused by the virus. Typically, following the administration or all of the administrations of the disclosed pharmaceutical formulation, an effective amount of the green tea catechin derivative (such as EC16) is administered to the subject to repair neurological damage caused by exposure to the virus, such as a SARS-CoV-2. For example, following the administration or all of the administration of the disclosed pharmaceutical formulation, the subject shows ameliorated or less symptoms or none of the symptoms associated with neurological damages caused by exposure to the virus, compared to the subject prior to the administration or first administration.

For example, the subject can be a mammal who was infected by a SARS-CoV-2 and shows long COVID symptoms after recovery (as indicated by a negative test result for SARS-CoV-2), including headache, persistent loss of smell, persistent loss of taste, memory loss, brain fog (such as difficulty concentrating, sense of confusion, and/or disorientation), dizziness, anxiety, depression, earache, hearing loss, ringing in ears (tinnitus), fatigue, and/or gastrointestinal issues. Following the administration or all of the administrations of the disclosed pharmaceutical formulation to the recovered subject, an effective amount of the green tea catechin derivative (such as EC16) is administered to the subject to ameliorate one or more of the long COVID symptoms. For example, following the administration or all of the administrations of the disclosed pharmaceutical formulation, one or more of the long COVID symptoms in the subject is/are ameliorated or disappear(s), compared to the subject prior to the administration or first administration. For example, by promoting cell differentiation, reducing inflammation, and/or reducing oxidative stress of nasal epithelial cells caused by exposure to a virus, for example a respiratory virus such as a SARS-CoV-2, the disclosed pharmaceutical formulations can restore the olfactory function of these cells.

### C. Exemplary Viruses

In preferred embodiments, the virus is a respiratory virus such as an influenza virus (e.g., influenza A, influenza virus B, or influenza virus C), respiratory syncytial virus (RSV), human metapneumovirus, parainfluenza virus, adenovirus, rhinovirus, or a coronaviruses. In some embodiments, the coronavirus is a betacoronavirus, such as Human Coronavirus OC43 (HCoV-OC43). In particularly preferred embodiments, the virus is a Severe acute respiratory syndrome-related virus, such as, SARSr-CoV BtKY72, SARS-CoV-2, SARS-CoV RaTG13, SARS-CoV PC4-227, or SARS-CoV, preferably one that infects humans such as SARS-CoV or SARS-CoV-2. In some embodiments, the virus is a Middle East respiratory syndrome-related virus such as MERS-CoV.

In the most preferred embodiments, the virus is a SARS-CoV-2. The sequence WIV04/2019, belonging to the GISAID S clade/PANGO A lineage/Nextstrain 19B clade, is thought to most closely reflect the sequence of the original SARS-CoV-2 infecting humans. It is known as “sequence zero”, and is widely used as a reference sequence. Subsequent to the initial isolate from Wuhan, China, numerous SARS-CoV-2 virus sequence variants of WIV04/2019 have been identified, some of which may be of particular importance due to their potential for increased transmissibility, increased virulence, and reduced effectiveness of vaccines against them. SARS-CoV-2 having a variation of the WIV04/2019 sequence include, but are not limited to:

B.1.1.7 lineage (a.k.a. 201/501Y.V1 Variant of Concern (VOC) 202012/01). This variant has a mutation in the receptor binding domain (RBD) of the spike protein at position 501, where the amino acid asparagine (N) has been replaced with tyrosine (Y). The shorthand for this mutation is N501Y. This variant also has several other mutations, including:

69/70 deletion: occurred spontaneously many times and likely leads to a conformational change in the spike protein.

P681H: near the S1/S2 furin cleavage site, a site with high variability in coronaviruses. This mutation has also emerged spontaneously multiple times.

B.1.351 lineage (a.k.a. 20H/501Y.V2). This variant has multiple mutations in the spike protein, including K417N, E484K, N501Y. Unlike the B.1.1.7 lineage detected in the UK, this variant does not contain the deletion at 69/70.

P.1 lineage (a.k.a. 20J/501Y.V3). The P.1 variant is a branch off the B.1.1.28 lineage that was first reported by the National Institute of Infectious Diseases (NIID) in Japan in four travelers from Brazil, sampled during routine screening at Haneda airport outside Tokyo. The P.1 lineage contains three mutations in the spike protein receptor binding domain: K417T, E484K, and N501Y.

Other in some embodiments, the SARS-CoV-2 is another two-mutation (e.g., N501T-G142D), or three-mutation (e.g., N501T-G142D-F486L) variants in the Spike protein.

In some embodiments, the SARS-CoV-2 is of the B.1.1.7 or a Q lineage (Alpha), B.1.351 or a descendent lineage thereof (Beta), P.1 or a descendent lineage thereof (Gamma), B.1.617.2 or an AY lineages (Delta) B.1.1.207, B.1.429 or B.1.427 (Epsilon), B.1.525 (Eta) lineage, B.1.617.1 (Kappa), 1.617.3, B.1.621 or B.1.621.1 (Mu), P.2 (Zeta), or a B.1.1.529, BA.1, BA.1.1, BA.2, BA.3, BA.4 or BA.5 lineage (Omicron). See also, “SARS-CoV-2 Variant Classifications and Definitions”, the Centers for Disease Control website, updated Apr. 26, 2022.

All of these lineages and sequence alternatives and variants relative to the WIV04/2019 strain are SARS-CoV-2 viruses.

In some embodiments, SARS-CoV-2 is a strain or isolate with elevated or potentially elevated risk for causing human disease relative to WIV04/2019. See, e.g., Science Brief, Emerging SARS-CoV-2 variants (CDC website, Updated Jan. 28, 2021), Horby, et al., “NERVTAG note on B.1.1.7 severity.” SAGE meeting report. Jan. 21, 2021; Wu, et al. “mRNA-1273 vaccine induces neutralizing antibodies against spike mutants from global SARS-CoV-2 variants.” bioRxiv. Posted Jan. 25, 2021; Xie, et al., “Neutralization of N501Y mutant SARS-CoV-2 by BNT162b2 vaccine-elicited sera.” bioRxiv. Posted Jan. 7, 2021; Greaney, et al. “Comprehensive mapping of mutations to the SARS-CoV-2 receptor-binding domain that affect recognition by polyclonal human serum antibodies.” bioRxiv. [Preprint posted online Jan. 4, 2021]; Weisblum, et al., “Escape from neutralizing antibodies by SARS-CoV-2 spike protein variants.” eLife 2020; 9:e61312; Resende, at al. “Spike E484K mutation in the first SARS-CoV-2 reinfection case confirmed in Brazil,” 2020. [Posted on virological.org on Jan. 10, 2021].

Any strain or isolate of SARS-CoV-2 can be treated with the disclosed compositions and according to the disclosed methods, including, but not limited to, those discussed in Coronaviridae Study Group of the International Committee on Taxonomy of Viruses, Nat Microbiol 2020. DOI: 10.1038/s41564-020-0695-z), and NCBI and GISAID which collectively provide hundreds of SARS-CoV-2 sequences.

An exemplary SARS-CoV-2 spike protein sequence is:

(SEQ ID NO: 1)
MFVFLVLLPLVSSQCVNLITRTQLPPAYTNSFTRGVYYPDKVFRSSVLHSTQDLFLPFFSNVTWFHAI [symbol] S
GTNGTKRFDNPVLPFNDGVYFASTEKSNIIRGWIFGTTLDSKTQSLLIVNNATNVVIKVCEFQFCNDPFL [symbol]
VYYHKNNKSWMESEFRVYSSANNCTFEYVSQPELMDLEGKQGNEKNLREFVEKNIDGYFKIYSKIITINLV
RDLPQGFSALEPLVDLPIGINITRFQTLLALHRSYLTPGDSSSGWTAGAAAYYVGYLQPRTFLLKYNENGT
ITDAVDCALDPLSETKCTLKSFTVEKGIYQTSNERVQPTESIVRFPNITNLCPFGEVENATRFASVYAWNR
KRISNCVADYSVLYNSASFSTFKCYGVSPTKLNDLCFTNVYADSFVIRGDEVRQIAPGQTG [symbol] IADYNYKLP
DDFTGCVIAWNSNNLDSKVGGNYNYLYRLERKSNLKPFERDISTEIYQAGSTPCNGV [symbol] G [symbol] NCYFPLQSYGF
QPT [symbol] GVGYQPYRVVVLSFELLHAPATVCGPKKSTNLVKNKCVNFNFNGLTGTGVLTESNKKFLPFQQFGRD
IADTTDAVRDPQTLEILDITPCSFGGVSVITPGTNTSNQVAVLYQDVNCTEVPVAIHADQLTPIWRVYSTG
SNVFQTRAGCLIGAEHVNNSYECDIPIGAGICASYQTQTNSPRRARSVASQSIIAYTMSLGAENSVAYSNN
SIAIPTNFTISVTTEILPVSMTKTSVDCTMYICGDSTECSNLLLQYGSFCTQLNRALTGIAVEQDKNTQEV
FAQVKQIYKTPPIKDFGGFNFSQILPDPSKPSKRSFIEDLLENKVTLADAGFIKQYGDCLGDIAARDLICA
QKFNGLTVLPPLLIDEMIAQYTSALLAGTITSGWTFGAGAALQIPFAMQMAYRENGIGVTQNVLYENQKLI
ANQFNSAIGKIQDSLSSTASALGKLQDVVNQNAQALNTLVKQLSSNFGAISSVLNDILSRLDKVEAEVQID
RLITGRLQSLQTYVTQQLIRAAEIRASANLAATKMSECVLGQSKRVDFCGKGYHLMSFPQSAPHGVVFLHV
TYVPAQEKNFTTAPAICHDGKAHFPREGVFVSNGTHWFVTQRNFYEPQIITTDNTFVSGNCDVVIGIVNNT
VYDPLQPELDSFKEELDKYFKNHTSPDVDLGDISGINASVVNIQKEIDRLNEVAKNLNESLIDLQELGKYE
QYIKWPWYIWLGFIAGLIAIVMVTIMLCCMTSCCSCLKGCCSCGSCCKFDEDDSEPVLKGVKLHYT,
which is encoded by 21563 . . . 25384 of GenBank: MN908947.3, /gene =
“S”, /note = “structural protein”, /codon_start = 1 /product = “surface
glycoprotein”, /protein_id = “QHD43416.1”).

In some embodiments, the spike protein of the SARS-CoV-2 has at least 70%, 75%, 80%, 85%, 86%, 87%, 88%, 89%, 90%, 91%, 92%, 93%, 94%, 95%, 96%, 97%, 98%, 99%, or 100% sequence identity to SEQ ID NO:1.

Mutations can be substitutions, insertions, deletions, or a combination thereof. Exemplary mutations include those discussed herein, e.g., one or more of the following mutations in the spike protein sequence:
H69, optionally deletion thereof;
V70, optionally deletion thereof;
G142, optionally G142D;
K417, optionally K417N or K417T;
E484, optionally E484K;
F486, optionally F486L; and/or
N501 mutation, optionally N501Y or N501T.
   These mutations are provided individually and in all combinations. These residues are illustrated with bold/italics/shading in SEQ ID NO:1 above.

In some embodiments, the subject was diagnosed with a positive SARS-CoV-2 viral test result and has or had at least one mild or moderate COVID-19 symptom (i.e. fever or chills, cough, shortness of breath or difficulty breathing, fatigue, muscle or body aches, headache, new loss of taste or smell, sore throat, congestion or runny nose, nausea, vomiting or diarrhea).

See also U.S. Published Application No. 2021/0196673, which are specifically incorporated by reference herein in its entirety.

The disclosed pharmaceutical formulations, devices, kits, and method of using can be further understood through the following numbered paragraphs.
1. A pharmaceutical formulation for nasal administration, comprising:
   a green tea catechin derivative;
   a carbohydrate; and
   a pharmaceutically acceptable carrier,
   wherein the pharmaceutical formulation has a viscosity sufficient to maintain the green tea catechin derivative in contact with nasal epithelial cells for a therapeutically effective amount of time, optionally for at least about 30 minutes in the presence of mucus.
2. The pharmaceutical formulation of paragraph 1, wherein the pharmaceutical formulation has a viscosity of at least about 15 cps, at least about 20 cps, at least about 25 cps, or at least about 30 cps and up to about 65 cps, such as in a range from about 15 cps to about 150 cps, from about 15 cps to about 125 cps, from about 15 cps to about 150 cps, from about 15 cps to about 95 cps, from about 15 cps to about 75 cps, from about 15 cps to about 65 cps, from about 20 cps to about 65 cps, from about 25 cps to about 65 cps, from about 30 cps to about 65 cps, from about 35 cps to about 65 cps, from about 40 cps to about 65 cps, from about 45 cps to about 65 cps, from about 30 cps to about 60 cps, from about 35 cps to about 60 cps, from about 40 cps to about 60 cps, from about 35 cps to about 55 cps, from about 40 cps to about 55 cps, or from about 45 cps to about 55 cps, such as about 50 cps.
3. The pharmaceutical formulation of paragraph 1 or 2, wherein the green tea catechin derivative is a derivative of epigallocatechin-3-gallate, epicatechin, epigallocatechin, or epicatechin-3-gallate, or a combination thereof.
4. The pharmaceutical formulation of any one of paragraphs 1-3, wherein the green tea catechin derivative is represented by Formula I:

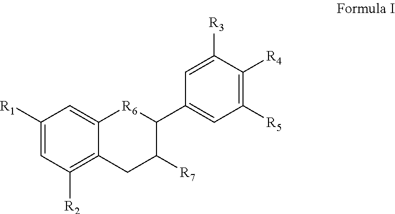

wherein:
(i) R₁-R₅ and R₇ are independently hydrogen, hydroxyl,

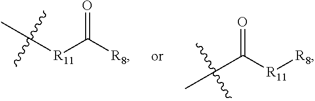

R₈ is a substituted C₁-C₃₀ alkyl, an unsubstituted C₁-C₃₀ alkyl, a substituted C₂-C₃₀ alkenyl, an unsubstituted C₂-C₃₀ alkenyl, a substituted C₂-C₃₀ alkynyl, an unsubstituted C₂-C₃₀ alkynyl, a substituted aryl, an unsubstituted aryl, a substituted polyaryl, or an unsubstituted polyaryl;
(ii) R₆ and R₁₁ is independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; and
(iii) at least one of R₁-R₅ and R₇ is

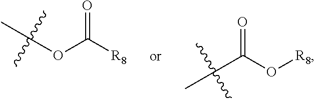

or
a pharmaceutically acceptable salt thereof.
5. The pharmaceutical formulation of any one of paragraphs 1-4, wherein the green tea catechin derivative is represented by Formula II:

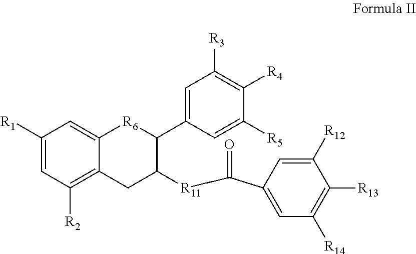

wherein:
(i) R₁-R₅ and R₁₂-R₁₄ are independently hydrogen, hydroxyl,

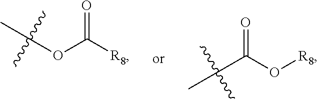

R₈ is a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl;
(ii) R₆ and R₁₁ are independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, or an unsubstituted C₁-C₃₀ alkynyl; and
(iii) least one of R₁-R₅ and R₁₂-R₁₄ is

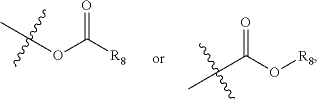

or
a pharmaceutically acceptable salt thereof.
6. The pharmaceutical formulation of paragraph 4 or 5, wherein R₆ and/or R₁₁ are oxygen.
7. The pharmaceutical formulation of any one of paragraphs 4-6, wherein R₁ and R₂ are hydroxyl.
8. The pharmaceutical formulation of any one of paragraphs 4-7, wherein at least one of R₃-R₅ is

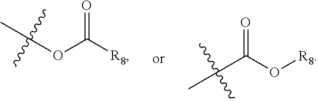

9. The pharmaceutical formulation of any one of paragraphs 5-8, wherein R₁₂-R₁₄ are independently hydroxyl,

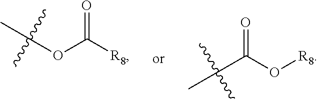

10. The pharmaceutical formulation of any one of paragraphs 4-9, wherein R₈ is a linear or branched substituted C₁-C₃₀ alkyl or a linear or branched unsubstituted C₁-C₃₀ alkyl.
11. The pharmaceutical formulation of paragraph 10, wherein R₈ is a linear or branched substituted C₁₄-C₂₅ alkyl or a linear or branched unsubstituted C₁₄-C₂₅ alkyl.
12. The pharmaceutical formulation of paragraph 11, wherein R₈ is C₁₅H₃₁.
13. The pharmaceutical formulation of any one of paragraphs 1-12, wherein the green tea catechin derivative is an epigallocatechin-3-gallate-palmitate, preferably wherein the green tea catechin derivative has the structure of:

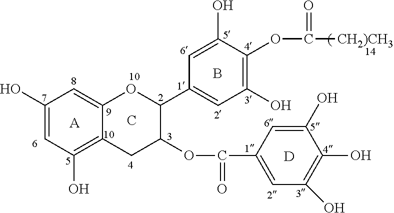

14. The pharmaceutical formulation of any one of paragraphs 1-13, wherein the carbohydrate is a polysaccharide, and wherein the polysaccharide is a cellulose, a derivative thereof, or a salt thereof, or a combination thereof.
15. The pharmaceutical formulation of paragraph 14, wherein the polysaccharide is selected from the group consisting of carboxymethylcellulose sodium, microcrystalline cellulose, xyloglucan, dendrobium officinale polysaccharide (DOP), hypomellose, glyceryl polymethacrylate, xanthan gum, hydroxyethylcellulose, guar gum, locust bean gum, carboxymethylcellulose, and hydroxypropylmethylcellulose.
16. The pharmaceutical formulation of any one of paragraphs 1-15, wherein the pharmaceutical formulation further comprises an emulsifier, a dispersing agent, an emollient, a buffering agent, a thickening agent, a chelating agent, or a preservative, or a combination thereof.
17. The pharmaceutical formulation of paragraph 16, wherein the emulsifier is a metaphosphate or salt thereof, polysorbate 80, glycerin, propolene glycol, or povidone, or a combination thereof.
18. The pharmaceutical formulation of paragraph 16 or 17, wherein the emollient is eucalyptol, glycerin, or propolene glycol, or a combination thereof.
19. The pharmaceutical formulation of any one of paragraphs 16-18, wherein the buffering agent is citric acid, acetic acid, acetate, hydrogen phosphate, dihydrogen phosphate, carbonate, bicarbonate, borate, or N-cyclohexyl-2-aminoethanesulfonic acid, or a combination thereof.
20. The pharmaceutical formulation of any one of paragraphs 16-19, wherein the thickening agent is carbomers, polyvinyl alcohol, povidone, colloidal silicon dioxide, cetyl alcohols, stearic acid, beeswax, petrolatum, triglycerides, or lanolin, or a combination thereof.
21. The pharmaceutical formulation of any one of paragraphs 16-20, wherein the chelating agent is ethylenediaminetetraacetic acid (EDTA) or ethylene glycol-bis(-aminoethyl ether)-N,N,N′,N′-tetraacetic acid (EGTA), or a combination thereof.
22. The pharmaceutical formulation of any one of paragraphs 16-21, wherein the preservative is benzalkonium chloride or potassium sorbate, or a combination thereof.
23. The pharmaceutical formulation of any one of paragraphs 1-22, wherein the pharmaceutical formulation further comprises an anti-inflammatory agent, an antioxidant, or antiviral agent, or a combination thereof.
24. The pharmaceutical formulation of any one of paragraphs 1-23, wherein the green tea catechin derivative is present in an amount from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation, or in a concentration from about 5 μM to about 5 mM, from about 10 μM to about 1.5 mM, from about 12.5 μM to about 1.25 mM, from about 12.5 μM to about 50 μM, or from about 50 μM to about 1.25 mM.
25. The pharmaceutical formulation of any one of paragraphs 1-24, wherein the carbohydrate is present in an amount from about 0.01% (w/v) to about 8% (w/v), from about 0.1% (w/v) to about 8% (w/v), from about 0.5% (w/v) to about 8% (w/v), from about 1% (w/v) to about 8% (w/v), from about 2% (w/v) to about 8% (w/v), from about 0.01% (w/v) to about 6% (w/v), from about 0.1% (w/v) to about 6% (w/v), from about 0.5% to about 6% (w/v), from about 1% to about 6% (w/v), from about 2% to about 6% (w/v), from about 0.01% (w/v) to about 4% (w/v), from about 0.1% (w/v) to about 4% (w/v), from about 0.5% to about 4% (w/v), from about 1% to about 4% (w/v), or from about 2% (w/v) to about 4% (w/v) of the pharmaceutical formulation.
26. The pharmaceutical formulation of any one of paragraphs 1-25, wherein the pharmaceutical formulation is an emulsion, optionally wherein the pharmaceutical formulation is a nanoemulsion.
27. The pharmaceutical formulation of paragraph 26, wherein the pharmaceutical formulation comprises at least two pharmaceutically acceptable carriers, wherein a first pharmaceutically acceptable carrier is an aqueous solution or water (such as distilled water, deionized water, and/or tap water), and wherein a second pharmaceutically acceptable carrier is an oil.
28. The pharmaceutical formulation of paragraph 27, wherein the oil forms oil droplets dispersed in the aqueous solution or water, and wherein the green tea catechin derivative is encapsulated in the oil droplets.
29. The pharmaceutical formulation of paragraph 28, wherein the oil droplets have an average diameter of less than 1 micron, less than or equal to about 900 nm, less than or equal to about 800 nm, less than or equal to about 700 nm, less than or equal to about 600 nm, less than or equal to about 500 nm, less than or equal to about 400 nm, less than or equal to about 300 nm, less than or equal to about 200 nm, less than or equal to about 150 nm, less than or equal to about 100 nm, or less than or equal to about 50 nm, such as in a range from about 100 nm to about 600 nm, from about 150 nm to about 600 nm, from about 200 nm to about 600 nm, from about 250 nm to about 600 nm, from about 300 nm to about 600 nm, from about 300 nm to about 500 nm, or from about 300 nm to about 400 nm.
30. The pharmaceutical formulation of any one of paragraphs 27-29, wherein the oil is present in an amount up to about 10 vol % or up to about 5 vol %, such as in a range from about 0.1 vol % to about 10 vol %, from about 0.5 vol % to about 10 vol %, from about 1 vol % to about 10 vol %, from about 0.1 vol % to about 5 vol %, from about 0.5 vol % to about 5 vol %, from about 1 vol % to about 5 vol %, from about 0.1 vol % to about 4 vol %, from about 0.5 vol % to about 4 vol %, from about 1 vol % to about 4 vol %, or from about 0.1 vol % to about 2 vol % of the pharmaceutical formulation.
31. The pharmaceutical formulation of any one of paragraphs 27-30, wherein the oil is soybean oil, mineral oil, avocado oil, squalene oil, olive oil, canola oil, corn oil, rapeseed oil, safflower oil, sunflower oil, fish oil, flavor oil, cinnamon bark, coconut oil, cottonseed oil, flaxseed oil, pine needle oil, silicon oil, essential oils, water insoluble vitamins, or phosphoglyceride, or a combination thereof.
32. The pharmaceutical formulation of any one of paragraphs 27-31, wherein the aqueous solution is a buffered aqueous solution, such as a phosphate-buffered saline (PBS) solution, a citrate buffer solution, MEM medium, phosphate buffer saline, and/or Hanks balanced salt solution.
33. The pharmaceutical formulation of any one of paragraphs 26-32, wherein the pharmaceutical formulation further comprises at least one emulsifier, optionally wherein the emulsifier is a non-ionic emulsifier selected from the group consisting of poloxamer, polysorbate, Triton® X-100, or nonoxynol-9, or a combination thereof; and/or a cationic emulsifier selected from the group consisting of cetylpyridimium chloride, benzalkonium chloride, benzethonium chloride, dioctadecyl dimethyl ammonium chloride, or octenidine dihydrochloride, or a combination thereof.
34. The pharmaceutical formulation of any one of paragraphs 26-33, wherein the pharmaceutical formulation further comprises a third pharmaceutically acceptable carrier that is an organic solvent, optionally wherein the organic solvent is ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohol, isopropanol, n-propanol, formic acid, propylene glycol, glycerol, sorbitol, industrial methylated spirit, triacetin, hexane, benzene, toluene, diethyl ether, chloroform, 1,4-dioxane, tetrahydrofuran, dichloromethane, acetone, acetonitrile, dimethylformamide, dimethyl sulfoxide, or formic acid, or a combination thereof.
35. The pharmaceutical formulation of any one of paragraphs 1-25, wherein the pharmaceutical formulation is an aqueous suspension.
36. The pharmaceutical formulation of paragraph 35, wherein the green tea catechin derivative is in the form of nanoparticles.
37. The pharmaceutical formulation of paragraph 36, wherein the particles have an average diameter of <5 μm, <4 μm, <3.5 μm, <3 μm, <2.5 μm, <2 μm, in a range from about 20 nm to about 1 μm, from about 50 nm to about 1 μm, or from about 100 nm to about 1 μm.
38. The pharmaceutical formulation of paragraph 36 or 37, wherein the particles have a fine particle fraction (the weight percentage of particles with a diameter less than 5 μm relative to the total weight of particles in the pharmaceutical formulation) of ≥90%, ≥95%, ≥98%, or ≥99%.
39. The pharmaceutical formulation of any one of paragraphs 35-38, wherein the pharmaceutically acceptable carrier is water, saline, phosphate buffered saline, a citrate buffer solution, EMEM medium, MEM medium, or Hanks balanced salt solution, or a combination thereof.
40. The pharmaceutical formulation of any one of paragraphs 1-39, wherein the pharmaceutical formulation has a pH in a range from 3 to 7.4, from 4 to 7, from 4 to 6, from 4 to 5, from 5 to 7, or from 5 to 6, such as about 6.
41. The pharmaceutical formulation of any one of paragraphs 1-39, wherein the pharmaceutical formulation is isotonic or hypertonic.
42. A delivery device comprising an inhaler and the pharmaceutical formulation of any one of paragraphs 1-41.
43. The delivery device of paragraph 42, wherein the inhaler is a pressurized metered dose inhaler that is configured to deliver a unit dosage of the pharmaceutical formulation per puff.
44. The delivery device of paragraph 43, wherein the amount of the green tea catechin derivative in the unit dosage is in a range from about 0.01 mg to about 20 mg, from about 0.01 mg to about 10 mg, from about 0.05 mg to about 20 mg, from about 0.05 mg to about 10 mg, from about 0.1 mg to about 20 mg, from about 0.1 mg to about 10 mg, from about 0.1 mg to about 5 mg, from about 0.5 mg to about 20 mg, from about 0.5 mg to about 10 mg, from about 0.5 mg to about 5 mg, from about 1 mg to about 20 mg, or from about 1 mg to about 10 mg.
45. A kit comprising one or more nasal swabs and the pharmaceutical formulation of any one of paragraphs 1-41.
46. The kit of paragraph 45, wherein the nasal swab and the pharmaceutical formulation are packaged separately.
47. The kit of paragraph 45, wherein the nasal swab is impregnated or saturated with the pharmaceutical formulation.
48. The kit of any one of paragraphs 45-47, wherein the nasal swab is sterile.
49. A method for preventing or reducing neurological damage caused by exposure to a virus in a subject in need thereof, comprising:
   (i) administering the pharmaceutical formulation of any one of paragraphs 1-41 to the nasal vestibule or passages of the subject.
50. The method of paragraph 49, wherein the subject is exposed to or is expected to be exposed to an individual having one or more symptoms of a viral infection, such as with fever, cough, shortness of breath, diarrhea, sneezing, runny nose, and/or sore throat.
51. The method of paragraph 49 or 50, wherein the administration is performed before and/or after the subject is exposed to the virus.
52. The method of paragraph 51, wherein the administration is performed within 24 hours, within 18 hours, within 12 hours, within 6 hours, within 4 hours, within 2 hours, or within 1 hour after the subject is tested positive for a viral infection (counting from the time point that positive test result is received by the subject).
53. The method of any one of paragraphs 49-52, wherein the method comprises repeating step (i).
54. The method of paragraph 53, wherein the administration step is repeated once, twice, or three times, per day, for a period of one day, three days, one week, two weeks, one month, three months, six months, or one year.
55. The method of any one of paragraphs 49-54, wherein following the administration or all of the administrations, the subject shows no or less symptoms associated with neurological damages caused by exposure to the virus, compared to a control.
56. The method of any one of paragraphs 49-55, wherein the virus is a respiratory virus, preferably a SARS-CoV-2 optionally B.1.617.2 (Delta) and/or B.1.1.529 (Omicron).
57. The method of paragraph 56, wherein the symptoms associated with neurological damages caused by exposure to the respiratory virus such as SARS-CoV-2 are selected from the group consisting of headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
58. The method of any one of paragraphs 49-57, wherein the administration is performed using a pressurized metered dose inhaler or a nasal swap, and wherein following the administration or each administration, a unit dosage is delivered to the nasal epithelial cells of the subject.
59. The method of any one of paragraphs 49-58, wherein the method further comprises administering an anti-inflammatory agent, an antioxidant, or antiviral agent, or a combination thereof to the subject, optionally by oral administration, nasal administration, and/or intravenous administration.
60. A method for repairing nasal epithelial cells, neuroepithelial cells, vascular cells, and/or neurons damaged by a virus in a subject in need thereof, comprising:
   (i) administering the pharmaceutical formulation of any one of paragraphs 1-41 to the nasal vestibule or passages of the subject.
61. The method of paragraph 60, wherein the administration is performed after the subject is recovered from a viral infection, as indicated by a negative test result for the virus.
62. The method of paragraph 61, wherein the subject shows one or more symptoms associated with neurological damages caused by exposure to the virus.
63. The method of paragraph 61 or 62, wherein the virus is a respiratory virus such as a SARS-CoV-2 optionally B.1.617.2 (Delta) and/or B.1.1.529 (Omicron).
64. The method of paragraph 63, wherein the symptoms associated with neurological damages caused by exposure to the respiratory virus such as the SARS-CoV-2 are selected from the group consisting of headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
65. The method of any one of paragraphs 60-64, wherein the method comprises repeating step (i).
66. The method of paragraph 65, wherein the administration step is performed once, twice, or three times, per day, for a period of one day, three days, one week, two weeks, one month, three months, six months, or one year.
67. The method of any one of paragraphs 62-66, wherein following the administration or all of the administrations, the subject shows less or none of the symptoms associated with neurological damages caused by exposure to the virus, compared to the subject prior to the administration or first administration.
68. The method of any one of paragraphs 60-67, wherein the administration is performed using a pressurized metered dose inhaler or a nasal swap, and wherein following the administration or each administration, a unit dosage is delivered to the nasal epithelial cells of the subject.
69. The method of any one of paragraphs 60-68, wherein the method further comprises administering an anti-inflammatory agent, an antioxidant, or antiviral agent, or a combination thereof to the subject, optionally by oral administration, nasal administration, and/or intravenous administration.
70. A method for treating anosmia in a subject in need thereof, comprising:
   (i) administering the pharmaceutical formulation of any one of paragraphs 1-41 to the nasal vestibule or passages of the subject.
71. A method for treating long COVID in a subject in need thereof, comprising:
   (i) administering the pharmaceutical formulation of any one of paragraphs 1-41 to the nasal vestibule or passages of the subject.
72. The method of paragraph 71, the long COVID comprises one or more symptoms selected from the group consisting of headache, persistent loss of smell (i.e. anosmia), persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
73. The method of any one of paragraphs 70-72, wherein the subject was exposed to, is exposed to, or is expected to be exposed to an individual having one or more symptoms of COVID infection, such as with fever, cough, shortness of breath, diarrhea, sneezing, runny nose, and/or sore throat.
74. The method of any one of paragraphs 70-73, wherein the administration is performed before the subject is exposed to a respiratory virus such as a SARS-CoV-2, after the subject is exposed to a respiratory virus such as a SARS-CoV-2, and/or after the subject is recovered from a COVID infection, as indicated by a negative test result for the virus.
75. The method of any one of paragraphs 70-74, wherein the administration is performed within 24 hours, within 18 hours, within 12 hours, within 6 hours, within 4 hours, within 2 hours, or within 1 hour after the subject is tested positive for a COVID infection (counting from the time point that positive test result is received by the subject).
76. The method of any one of paragraphs 70-75, wherein the method comprises repeating step (i).
77. The method of paragraph 76, wherein the administration step is repeated once, twice, or three times, per day, for a period of one day, three days, one week, two weeks, one month, three months, six months, or one year.
78. The method of any one of paragraphs 71-77, wherein following the administration or all of the administrations, the subject shows no or less symptoms associated with long COVID, compared to a control.
79. The method of any one of paragraphs 70-78, wherein following the administration or all of the administrations, the subject regain at least a portion of the sense of smell, compared to the subject before the administration or first administration.
80. The method of any one of paragraphs 70-79, wherein the administration is performed using a pressurized metered dose inhaler or a nasal swap, and wherein following the administration or each administration, a unit dosage is delivered to the nasal epithelial cells of the subject.
81. The method of any one of paragraphs 70-80, wherein the method further comprises administering an anti-inflammatory agent, an antioxidant, or antiviral agent, or a combination thereof to the subject, optionally by oral administration, nasal administration, and/or intravenous administration.
82. A method for reducing viral infection, comprising administering the pharmaceutical formulation of any one of paragraphs 1-41 to a subject in need thereof.
83. The method of paragraph 82, wherein the subject has not been exposed to the virus.
84. The method of paragraph 82, wherein the subject has been exposed to the virus.
85. The method of paragraph 84, wherein the subject is infected with the virus.
86. The method of any one of paragraphs 82-85, wherein the pharmaceutical formulation is administered to the nasal vestibule or passages of the subject.
87. The method of any one of paragraphs 82-86, wherein the virus is a respiratory virus.
88. The method of paragraph 87, wherein the respiratory virus is selected from the group consisting of influenza virus (e.g., influenza A, influenza virus B, or influenza virus C), respiratory syncytial virus (RSV), human metapneumovirus, parainfluenza virus, adenovirus, rhinovirus, or a coronaviruses.
89. The method of paragraph 88, wherein the coronavirus is a betacoronavirus, optionally Human Coronavirus OC43 (HCoV-OC43), a Severe acute respiratory syndrome-related virus, optionally, SARS-CoV BtKY72, SARS-CoV-2, SARS-CoV RaTG13, SARS-CoV PC4-227, or SARS-CoV, preferably one that infects humans such as SARS-CoV or SARS-CoV-2, or a Middle East respiratory syndrome-related virus such as MERS-CoV.

The disclosed pharmaceutical formulations, devices, kits, and methods can be further understood through the following numbered paragraphs.
1. A pharmaceutical formulation for nasal administration, comprising:
   nanoparticles of a green tea catechin derivative; and
   a pharmaceutically acceptable carrier.
2. The pharmaceutical formulation of paragraph 1, wherein the green tea catechin derivative is a derivative of epigallocatechin-3-gallate, epicatechin, epigallocatechin, or epicatechin-3-gallate, or a combination thereof.
3. The pharmaceutical formulation of paragraph 1 or 2, wherein the green tea catechin derivative is represented by Formula I:

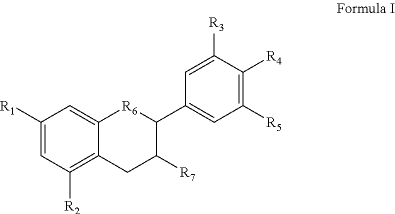

wherein:
(i) R₁-R₅ and R₇ are independently hydrogen, hydroxyl,

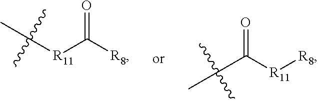

R₈ is a substituted C₁-C₃₀ alkyl, an unsubstituted C₁-C₃₀ alkyl, a substituted C₂-C₃₀ alkenyl, an unsubstituted C₂-C₃₀ alkenyl, a substituted C₂-C₃₀ alkynyl, an unsubstituted C₂-C₃₀ alkynyl, a substituted aryl, an unsubstituted aryl, a substituted polyaryl, or an unsubstituted polyaryl;
(ii) R₆ and R₁₁ is independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl; and
(iii) at least one of R₁-R₅ and R₇ is

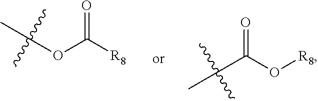

or
a pharmaceutically acceptable salt thereof.
4. The pharmaceutical formulation of any one of paragraphs 1-3, wherein the green tea catechin derivative is represented by Formula II:

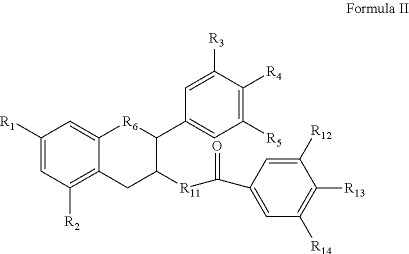

wherein:
(i) R₁-R₅ and R₁₂-R₁₄ are independently hydrogen, hydroxyl,

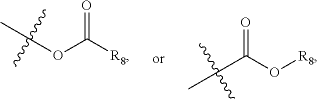

R₈ is a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, an unsubstituted C₁-C₃₀ alkynyl, a substituted aryl, an unsubstituted aryl, a substituted polyaryl, or an unsubstituted polyaryl;
(ii) R₆ and R₁₁ are independently oxygen, —NR₉R₁₀, or sulfur, R₉ and R₁₀ are independently hydrogen, a substituted C₁-C₃₀ alkyl, an unsubstituted C₁-C₃₀ alkyl, a substituted C₁-C₃₀ alkenyl, an unsubstituted C₁-C₃₀ alkenyl, a substituted C₁-C₃₀ alkynyl, or an unsubstituted C₁-C₃₀ alkynyl; and
(iii) least one of R₁-R₅ and R₁₂-R₁₄ is

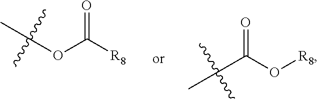

or
a pharmaceutically acceptable salt thereof.
5. The pharmaceutical formulation of paragraph 3 or 4, wherein R₆ and/or R₁₁ are oxygen.
6. The pharmaceutical formulation of any one of paragraphs 3-5, wherein R₁ and R₂ are hydroxyl.
7. The pharmaceutical formulation of any one of paragraphs 3-6, wherein at least one of R₃-R₅ is

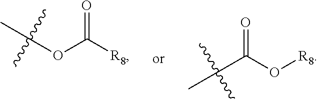

8. The pharmaceutical formulation of any one of paragraphs 4-7, wherein R₁₂-R₁₄ are independently hydroxyl,

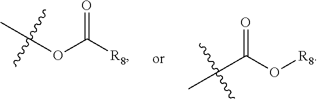

9. The pharmaceutical formulation of any one of paragraphs 3-8, wherein R₈ is a linear or branched substituted C₁-C₃₀ alkyl or a linear or branched unsubstituted C₁-C₃₀ alkyl.
10. The pharmaceutical formulation of paragraph 9, wherein R₈ is a linear or branched substituted C₁₄-C₂₅ alkyl or a linear or branched unsubstituted C₁₄-C₂₅ alkyl.
11. The pharmaceutical formulation of paragraph 10, wherein R₈ is C₁₅H₃₁.
12. The pharmaceutical formulation of any one of paragraphs 1-11, wherein the green tea catechin derivative is an epigallocatechin-3-gallate-palmitate, preferably wherein the green tea catechin derivative has the structure of:

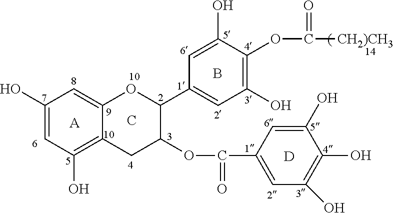

13. The pharmaceutical formulation of any one of paragraphs 1-12, wherein the nanoparticles have a median diameter of less than 500 nm, less than 400 nm, less than 300 nm, or less than 200 nm, such as ranging from about 10 nm to about 500 nm, from about 10 nm to about 400 nm, from about 10 nm to about 300 nm, from about 10 nm to about 200 nm, from about 10 nm to about 150 nm, from about 50 nm to about 500 nm, from about 50 nm to about 400 nm, from about 50 nm to about 300 nm, from about 50 nm to about 200 nm, or from about 50 nm to about 150 nm.
14. The pharmaceutical formulation of any one of paragraphs 1-13, wherein the green tea catechin derivative is present in an amount from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation.
15. The pharmaceutical formulation of any one of paragraphs 1-13, wherein the nanoparticles have a zeta potential in a range from about-80 mV to about-10 mV or from about-50 mV to about-20 mV.
16. The pharmaceutical formulation of any one of paragraphs 1-13, wherein the pharmaceutically acceptable carrier is a mixture of (a) glycerol and (b) water or an aqueous solution, and optionally (c) an alcohol; and optionally wherein the glycerol is present in an amount from 0.1 wt % to 99.5 wt %, from 0.1 wt % to 35 wt %, from 10 wt % to 99.5 wt % or from 40 wt % to 99.5 wt %; or the total of glycerol and ethylene glycol is present in an amount from 0.1 wt % to 99.5 wt %, from 0.1 wt % to 35 wt %, from 10 wt % to 99.5 wt %, or from 40 wt % to 99.5 wt % in the pharmaceutical formulation.
17. The pharmaceutical formulation of paragraph 15 or 16, wherein the water or aqueous solution is present in an amount from 0.1 wt % to 99 wt %, from 60 wt % to 99 wt %, from 0.1 wt % to 30 wt %, or from 0.1 wt % to 15 wt % in the pharmaceutical formulation.
18. The pharmaceutical formulation of any one of paragraphs 15-17, wherein the alcohol, when present, is in an amount ≤1 wt %, such as from about 0.1 wt % to about 0.3 wt %, in the pharmaceutical formulation.
19. The pharmaceutical formulation of any one of paragraphs 15-18, wherein the aqueous solution is a saline solution (normal, isotonic, or hypertonic), phosphate buffered saline, a citrate buffer solution, MEM medium, or Hanks balanced salt solution, such as a normal saline solution.
20. The pharmaceutical formulation of any one of paragraphs 15-19, wherein the alcohol is ethanol, n-propanol, or isopropanol, or a combination thereof.
21. The pharmaceutical formulation of any one of paragraphs 1-20, having a pH in a range from 3 to 8.5, from 3 to 7.4, from 4 to 7, from 4 to 6, from 4 to 5, from 5 to 7, from 5 to 6, or from 6.5 to 7.4.
22. The pharmaceutical formulation of any one of paragraphs 1-21, wherein the pharmaceutical formulation is isotonic or hypertonic.
23. The pharmaceutical formulation of any one of paragraphs 1-22, further comprising a dispersing agent, optionally wherein the dispersing agent is a carbohydrate or salt thereof (e.g., sodium carboxymethyl cellulose), or a metaphosphate (e.g., trimetaphosphate, hexametaphosphate, etc.) or salt thereof (e.g., sodium trimetaphosphate, sodium hexametaphosphate, etc.), or a combination thereof.
24. The pharmaceutical formulation of paragraph 23, wherein the dispersing agent is present in an amount from about 0.0005% (w/v) to about 1% (w/v), from about 0.005% (w/v) to about 1% (w/v), from about 0.05% (w/v) to about 1% (w/v), from about 0.005% (w/v) to about 0.5% (w/v), from about 0.01% (w/v) to about 0.5% (w/v), from about 0.1% (w/v) to about 0.5% (w/v), from about 0.005% (w/v) to about 2% (w/v), from about 0.01% (w/v) to about 2% (w/v), or from about 0.1% (w/v) to about 2% (w/v) in the pharmaceutical formulation.
25. The pharmaceutical formulation of paragraph 23 or 24, wherein the dispersing agent is sodium hexametaphosphate.
26. The pharmaceutical formulation of any one of paragraphs 1-25, further comprising an emulsifier, an emollient, a buffering agent, a thickening agent, a chelating agent, or a preservative, or a combination thereof.
27. The pharmaceutical formulation of any one of paragraphs 1-26, further comprising an anti-inflammatory agent, an antioxidant, or antiviral agent, or a combination thereof.
28. A delivery device comprising an inhaler and the pharmaceutical formulation of any one of paragraphs 1-27.
29. The delivery device of paragraph 28, wherein the inhaler is a pressurized metered dose inhaler that is configured to deliver a unit dosage of the pharmaceutical formulation per puff.
30. The delivery device of paragraph 29, wherein the amount of the green tea catechin derivative in the unit dosage is in a range from about 0.01 mg to about 20 mg, from about 0.01 mg to about 10 mg, from about 0.05 mg to about 20 mg, from about 0.05 mg to about 10 mg, from about 0.1 mg to about 20 mg, from about 0.1 mg to about 10 mg, from about 0.1 mg to about 5 mg, from about 0.5 mg to about 20 mg, from about 0.5 mg to about 10 mg, from about 0.5 mg to about 5 mg, from about 1 mg to about 20 mg, or from about 1 mg to about 10 mg.
31. A kit comprising one or more nasal swabs and the pharmaceutical formulation of any one of paragraphs 1-27.
32. The kit of paragraph 31, wherein the nasal swab and the pharmaceutical formulation are packaged separately.
33. The kit of paragraph 31, wherein the nasal swab is impregnated or saturated with the pharmaceutical formulation.
34. The kit of any one of paragraphs 31-33, wherein the nasal swab is sterile.
35. A method of making the pharmaceutical formulation of paragraph 1, comprising:
   (i) dissolving the green tea catechin derivative in a first pharmaceutically acceptable carrier to form a green tea catechin derivative solution; and
   (ii) mixing the green tea catechin derivative solution with a second pharmaceutically acceptable carrier to produce a suspension formulation comprising nanoparticles of the green tea catechin derivative.
36. The method of paragraph 35, further comprising (iii) mixing the suspension formulation with a third pharmaceutically acceptable carrier.
37. The method of paragraph 35 or 36, wherein the first pharmaceutically acceptable carrier is an alcohol or a water mixture thereof; the second pharmaceutically acceptable carrier is glycerol or a water mixture thereof; the third pharmaceutically acceptable carrier is water or an aqueous solution.
38. The method of any one of paragraphs 35-37, wherein the aqueous solution comprises a dispersing agent, such as a metaphosphate.
39. A method for preventing or reducing neurological damage caused by exposure to a virus in a subject in need thereof, comprising:
   (i) administering the pharmaceutical formulation of any one of paragraphs 1-27 to the nasal vestibule or passages of the subject.
40. The method of paragraph 39, wherein the subject is exposed to or is expected to be exposed to an individual having one or more symptoms of a viral infection, such as with fever, cough, shortness of breath, diarrhea, sneezing, runny nose, and/or sore throat.
41. The method of paragraph 39 or 40, wherein the administration is performed before and/or after the subject is exposed to the virus.
42. The method of paragraph 41, wherein the administration is performed within 24 hours, within 18 hours, within 12 hours, within 6 hours, within 4 hours, within 2 hours, or within 1 hour after the subject is tested positive for a viral infection (counting from the time point that positive test result is received by the subject).
43. The method of any one of paragraphs 39-42, wherein the method comprises repeating step (i).
44. The method of paragraph 43, wherein the administration step is repeated once, twice, or three times, per day, for a period of one day, three days, one week, two weeks, one month, three months, six months, or one year.
45. The method of any one of paragraphs 39-44, wherein following the administration or all of the administrations, the subject shows no or less symptoms associated with neurological damages caused by exposure to the virus, compared to a control.
46. The method of any one of paragraphs 39-45, wherein the virus is a respiratory virus such as SARS-CoV-2, optionally B.1.617.2 (Delta) and/or B.1.1.529 (Omicron).
47. The method of paragraph 46, wherein the symptoms associated with neurological damages caused by exposure to the respiratory virus such as the SARS-CoV-2 are selected from the group consisting of headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
48. The method of any one of paragraphs 39-47, wherein the administration is performed using a pressurized metered dose inhaler or a nasal swap, and wherein following the administration or each administration, a unit dosage is delivered to the nasal epithelial cells of the subject.
49. The method of any one of paragraphs 39-48, wherein the method further comprises administering an anti-inflammatory agent, an antioxidant, or antiviral agent, or a combination thereof to the subject, optionally by oral administration, nasal administration, and/or intravenous administration.
50. A method for repairing nasal epithelial cells, neuroepithelial cells, vascular cells, and/or neurons damaged by a virus in a subject in need thereof, comprising:
   (i) administering the pharmaceutical formulation of any one of paragraphs 1-27 to the nasal vestibule or passages of the subject.
51. The method of paragraph 50, wherein the administration is performed after the subject is recovered from a viral infection, as indicated by a negative test result for the virus.
52. The method of paragraph 51, wherein the subject shows one or more symptoms associated with neurological damages caused by exposure to the virus.
53. The method of paragraph 51 or 52, wherein the virus is a respiratory virus such as a SARS-CoV-2 optionally B.1.617.2 (Delta) and/or B.1.1.529 (Omicron).
54. The method of paragraph 53, wherein the symptoms associated with neurological damages caused by exposure to the respiratory virus such as the SARS-CoV-2 are selected from the group consisting of headache, persistent loss of smell, persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
55. The method of any one of paragraphs 50-54, wherein the method comprises repeating step (i).
56. The method of paragraph 55, wherein the administration step is performed once, twice, or three times, per day, for a period of one day, three days, one week, two weeks, one month, three months, six months, or one year.
57. The method of any one of paragraphs 52-56, wherein following the administration or all of the administrations, the subject shows less or none of the symptoms associated with neurological damages caused by exposure to the virus, compared to the subject prior to the administration or first administration.
58. The method of any one of paragraphs 50-57, wherein the administration is performed using a pressurized metered dose inhaler or a nasal swap, and wherein following the administration or each administration, a unit dosage is delivered to the nasal epithelial cells of the subject.
59. The method of any one of paragraphs 50-58, wherein the method further comprises administering an anti-inflammatory agent, an antioxidant, or antiviral agent, or a combination thereof to the subject, optionally by oral administration, nasal administration, and/or intravenous administration.
60. A method for treating anosmia in a subject in need thereof, comprising:
   (i) administering the pharmaceutical formulation of any one of paragraphs 1-27 to the nasal vestibule or passages of the subject.
61. A method for treating long COVID in a subject in need thereof, comprising:
   (i) administering the pharmaceutical formulation of any one of paragraphs 1-27 to the nasal vestibule or passages of the subject.
62. The method of paragraph 61, the long COVID comprises one or more symptoms selected from the group consisting of headache, persistent loss of smell (i.e. anosmia), persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.
63. The method of any one of paragraphs 60-62, wherein the subject was exposed to, is exposed to, or is expected to be exposed to an individual having one or more symptoms of COVID infection, such as with fever, cough, shortness of breath, diarrhea, sneezing, runny nose, and/or sore throat.
64. The method of any one of paragraphs 60-63, wherein the administration is performed before the subject is exposed to a respiratory virus such as a SARS-CoV-2, after the subject is exposed to a respiratory virus such as a SARS-CoV-2, and/or after the subject is recovered from a COVID infection, as indicated by a negative test result for the virus.
65. The method of any one of paragraphs 60-64, wherein the administration is performed within 24 hours, within 18 hours, within 12 hours, within 6 hours, within 4 hours, within 2 hours, or within 1 hour after the subject is tested positive for a COVID infection (counting from the time point that positive test result is received by the subject).
66. The method of any one of paragraphs 60-65, wherein the method comprises repeating step (i).
67. The method of paragraph 66, wherein the administration step is repeated once, twice, or three times, per day, for a period of one day, three days, one week, two weeks, one month, three months, six months, or one year.
68. The method of any one of paragraphs 61-67, wherein following the administration or all of the administrations, the subject shows no or less symptoms associated with long COVID, compared to a control.
69. The method of any one of paragraphs 60-68, wherein following the administration or all of the administrations, the subject regain at least a portion of the sense of smell, compared to the subject before the administration or first administration.
70. The method of any one of paragraphs 60-69, wherein the administration is performed using a pressurized metered dose inhaler or a nasal swap, and wherein following the administration or each administration, a unit dosage is delivered to the nasal epithelial cells of the subject.
71. The method of any one of paragraphs 60-70, wherein the method further comprises administering an anti-inflammatory agent, an antioxidant, or antiviral agent, or a combination thereof to the subject, optionally by oral administration, nasal administration, and/or intravenous administration.
72. A method for reducing viral infection, comprising administering the pharmaceutical formulation of any one of paragraphs 1-27 to a subject in need thereof.
73. The method of paragraph 72, wherein the subject has not been exposed to the virus.
74. The method of paragraph 72, wherein the subject has been exposed to the virus.
75. The method of paragraph 74, wherein the subject is infected with the virus.
76. The method of any one of paragraphs 72-75, wherein the pharmaceutical formulation is administered to the nasal vestibule or passages of the subject.
77. The method of any one of paragraphs 72-76, wherein the virus is a respiratory virus.
78. The method of paragraph 77, wherein the respiratory virus is selected from the group consisting of influenza virus (e.g., influenza A, influenza virus B, or influenza virus C), respiratory syncytial virus (RSV), human metapneumovirus, parainfluenza virus, adenovirus, rhinovirus, or a coronaviruses.
79. The method of paragraph 78, wherein the coronavirus is a betacoronavirus, optionally Human Coronavirus OC43 (HCoV-OC43), a Severe acute respiratory syndrome-related virus, optionally, SARSr-CoV BtKY72, SARS-CoV-2, SARS-CoV RaTG13, SARS-CoV PC4-227, or SARS-CoV, preferably one that infects humans such as SARS-CoV or SARS-CoV-2, or a Middle East respiratory syndrome-related virus such as MERS-CoV.

### EXAMPLES

### Example 1. Evaluation of Epigallocatechin-3-Gallate-Palmitate (EC16) in Nasal Formulations Against Human Coronavirus

### Materials and Methods

### Virus and Cell Line

OC43 human coronavirus (ATCC VR-1558) and MCR-5 human respiratory fibroblast cells (ATCC CCL-171) were purchased from ATCC.

### EC16 and Other Supplies

Epigallocatechin-3-Gallate-Palmitates (EC16; a mixture of mono-di- and tri-palmitates) and Epigallocatechin-3-Gallate-mono-palmitate (EC16m) were provided by Camellix, LLC (Evans, GA). Eagle's Minimal Essential Medium (EMEM) was purchased from ATCC (30-2003). Fetal bovine serum (FBS) was from Neuromics (Edina, MN). Trypsin (0.25%)-EDTA was provided by Fisher Scientific. Penicillin, streptomycin, and amphotericin B solution (100×) was ordered from Corning (Glendale, AR). Plasticwares were purchased from Southern Labware (Cumming, GA).

### EC16 Formulations

EC16 and EC16m were initially dispersed as stable glycerol-based stocks (F18 and F18m respectively) at 1% w/v. Working formulations were made by a 10× dilution with Eagle's Minimum Essential Medium (EMEM) (serum-free EMEM, also referred to herein as MM, for cell-based studies), normal saline solution (for future clinical studies), or phosphate buffer saline (PBS, for animal studies). These 0.1% EC16 or EC16m formulations were equal to approximately 1.25 mM (EC16) and 1.40 mM (EC16m), respectively, from which they were diluted to lower concentrations for the experiments. Two additional formulations (F18D and F18Dm) were prepared by diluting the stock 10× with normal saline. Sodium hexametaphosphate stock (10%) was added into the diluted suspension at a 1:10 ratio. The tests of components for exemplary formulations of EC16 and the observation is shown in Table 1. The test of control formulations without EC16 is shown in Table 2. Additionally, F18BC was formed from F18 basic formulation with 1% sodium carboxymethyl cellulose (CMC) and 0.1% EC16, and F18DC was formed from F18D formulation with 1% CMC and 0.1% EC16. F100S basic formulation is 0.04% w/v EC16, 0.138% ethanol, 31.7% glycerol (v/v), 66.1% saline (0.595% saline) (v/v), and 0.2% water or 0.2% HMP w/v in water for F100SD (Table 5).

TABLE 1
Tests of Components for Exemplary Formulations of EC16 and Observations

| Pharmaceutically | Dispersing | Formation of | Antiviral | | |
|---|---|---|---|---|---|
| Acceptable Carriers | Agent | Nanoparticles? | Activity* | Stability | Cytotoxicity |
| DMSO, water | PS80 | No | NA | Low | NA |
| ethanol, glycerol, | PA | Yes | Enhanced | Modest | Modest |
| normal saline | | | | | |
| ethanol, glycerol, | PVP | Yes | Reduced | Yes | High |
| normal saline | | | | | |
| ethanol, glycerol, | PVA | Yes | Reduced | Yes | High |
| normal saline | | | | | |
| ethanol, glycerol, | CMC | Yes | No change | Yes | Low |
| normal saline | | | | | |
| ethanol, glycerol, | SHMP | Yes | Enhanced** | Yes | Low |
| normal saline | | | | | |

*The antiviral activity was compared to a formulation containing the same components expect for the dispersing agent.
**The formulation containing SHMP showed the highest antiviral activity among all formulations tested.

TABLE 2
Test of Control Formulations without EC16

| Pharmaceutically | Dispersing | Antiviral | |
|---|---|---|---|
| Acceptable Carriers | Agent | activity | Cytotoxicity |
| saline | none | No | Moderate |
| glycerol | none | No | Low |
| normal saline | SHMP | No | Modest |
| glycerol | SHMP | No | Modest |
| glycerol, normal saline | SHMP | No | Modest |
| glycerol, normal saline | PVP | No | High |
| glycerol, normal saline | PVA | No | High |

### Quantitation of EC16 Polyphenol in Mixtures

After dilution of F18 in aqueous buffers, the flocculent material was separated by centrifugation (700 rpm for 3 min). The liquid under the cream was removed and the cream was reconstituted by vigorous vortexing in the same volume of liquid. The distribution of polyphenols in the fractions was determined by Folin-Ciocalteu reaction as described in Everette, et al. J Agric Food Chem 2010, 58, 8139-8144 with minor modifications, using EC16 dissolved in ethanol as the standard.

### Evaluation of Particle Size Distribution

Zeta View nanoparticle tracking analysis was performed according to a method described in Helwa, et al. PLOS One 2017, 12, e0170628. The particle size distribution and concentration were measured using the Zetaview ×20 (Particle Metrix, Meerbusch, Germany) and corresponding software. The measuring range for particle diameter is 10-2000 nm. The samples were diluted by the same volume of 1×PBS and then loaded into a compartment. The instrument collected particle information from 11 different positions across the compartment, with two cycles of readings respectively. The standard operating procedure was set to a temperature of 23° C., a sensitivity of 70, a frame rate of 30 frames per second, and a shutter speed of 100. The post-acquisition parameters were set to a minimum brightness of 20, a maximum area of 1000, a minimum area of 10, and a tracelength of 15.

### Antiviral Activity Tests

Infection of cells by OC43 virus, and viral titer: MRC-5 cells was cultured in EMEM Medium supplemented with 10% FBS and 1% penicillin, streptomycin, and amphotericin B. The viral infection assay and viral titering were performed in 96 well cell culture plates when the cells had reached 90% confluency. A 10-fold series dilution of OC43 virus in serum-free EMEM was loaded into wells in quadruplicates per dilution. After one-hour absorption, the viral dilutions were removed and 100 μl serum-free EMEM was added, followed by incubation at 33° C. with 5% CO₂ for >4 days to allow a CPE (cytopathic effect) to become visible. Viral titer was calculated by a TCID50 protocol and software (Reed, et al. American Journal of Epidemiology 1938, 27, 493-497). A minimum of three independent experiments were performed and results recorded. To compare EC16 activity to an antiviral in clinical use, exposure of cells to 50 μM remdesivir was performed.

### Antiviral Activity Tests of F18m

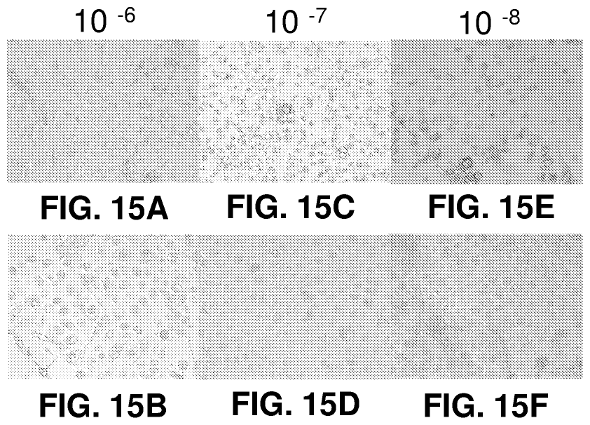

Formulation F18m was diluted 10× and 25× by basal medium (concentrations without cytotoxicity, see FIGS. 15A-15F). HNpEC were grown in 24-well plate, and then the cells were infected by OC43 virus in series dilutions for 10 minutes. Virus was removed and dilutions of F18m was placed in the wells for 1 h before medium-change. After 7 days, the plate was scored according to TCID50 assay and calculated.

HNpEC were grown in 24-well plate. Cells were infected by OC43 virus in series dilutions for 10 minutes. Virus was removed and dilutions of F18m was placed in the wells for 1 h before medium-change. After 7 days, the plate was scored according to TCID50 assay and calculated, and EVOS images were taken.

Dose and Time Tests of Direct Contact with Virus

EC16 nasal formulations were used with different concentrations or incubation times in direct contact with OC43 virus in serum free EMEM. To determine the dose effect of EC16, in three independent tests the F18 stock formulation was diluted with serum-free EMEM to a series of concentrations from 1.25 mM down to 0.05 mM, and incubated with OC43 (titer log 9.25/ml diluted in serum-free EMEM) for 30 min. To determine exposure effects at different times, a 1.25 mM dilution of F18 in serum-free EMEM was mixed with virus for 5, 15, 30 or 60 min, followed by rapid serial dilution with serum-free EMEM and loaded onto 96-well plates containing 90% confluent MRC-5 cells for one-hour viral absorption, followed by media change. For testing of F18D, the F18D EC16 nasal formulation was diluted with normal saline to a concentration of 1.25 mM EC16. This working formulation was incubated with OC43 virus at a 1:9 ratio (virus to formulation) for 1, 5, and 15 min before 10× serial dilutions and then subjected to TCID50 assay. The infectivity rate was determined by TCID50 method after 4-7 days of incubation.

### Pre-infection Test

EC16m nasal formulations were incubated with MRC-5 cells in 96 well plates for 10 min, and then removed. A series dilution of OC43 in serum-free EMEM was added to the cells and incubated for 1 h. The viral dilutions were then replaced with serum-free EMEM, and TCID50 infection rate was determined after 4-7 days of incubation.

### Post-Infection Test

To test if EC16m nasal formulations possess a post-infection effect, MRC-5 cells in 96 well cell culture plate were infected for one hour with the virus in series dilutions before removal. Then, 100 μl of either EC16 formulations or Remdesivir solutions of various concentrations were applied onto the cells for 10 min before being replaced by serum-free EMEM. The TCID50 values were determined after incubation for 4-7 days.

### Photograph of the Formulations

EC16 particles in the three F18 diluted suspension formulations (MM, PBS and saline) were photographed using transmitted light microscope at 40× magnification with DIC illumination under phase contrast, and the images were recorded.

### Cytotoxicity Studies of F18BC and F18DC

To determine cell viability of formulations F18BC and F18DC with saline-treated cells, F18BC was diluted 2, 5, 10, 15, 20 times by basal medium and F18DC diluted 2, 5, 10, 15, 20 times by basal medium (Table 3). Each dilution of F18BC and F18DC was incubated with HNpEC, after 60 minutes, and then the medium changed with Complete airway medium overnight. Next day, MTT assays were performed using CytoSelect MTT Cell Proliferation Assay kit (Cell Biolabs, Inc).

TABLE 3
Study Design of Cytotoxic Studies
using HNpEC for F18BC and F18DC

| F18 | | | | | | | | |
|---|---|---|---|---|---|---|---|---|
| BC | MC | C | 0 | 2 | 5 | 10 | 15 | 20 |
| BC | MC | C | 0 | 2 | 5 | 10 | 15 | 20 |
| BC | MC | C | 0 | 2 | 5 | 10 | 15 | 20 |
| DC | MC | C | 0 | 2 | 5 | 10 | 15 | 20 |
| DC | MC | C | 0 | 2 | 5 | 10 | 15 | 20 |
| DC | MC | C | 0 | 2 | 5 | 10 | 15 | 20 |

### Cytotoxic Studies of F18m and F18Dm in HCT-8 Cells

To determine cell viability of formulations F18m and F18Dm in HCT-8 cells, control formulations in saline with 10% glycerol, 1% SHMP in saline with 10% glycerol, and test formulations, F18 with 0.1% EC16m and 10% glycerol, saline, and F18D with 0.1% EC16m, 10% glycerol, SHMP, and saline, were made.

One 96-well plate was used with confluent HCT-8 cells, dilute each formulation with cell culture medium at 0, 1:1, 1:5 and 1:10 ratio before loading onto the wells with 3 repeats. The plate was incubated with the formulations for 60 minutes before medium change and overnight incubation. MTT assay was performed with the plate using 540 nm wavelength.

### Cytotoxicity Study of F18m in Normal Saline

The F18m glycerol stock was diluted 10× with normal saline to 1.4 mM of EC16m. A series dilution of this nanoformulation in Airway Growth Medium was applied to a monolayer of Human NaF18sal Primary Epithelial Cells (HNPEC, PromoCell) and incubated for 1 h before medium change and overnight incubation, followed by straining with an MTT assay.

### Cytotoxicity Studies of F18/F100 Formulations

To test for cytotoxicity, formulations and controls were prepared with a base of 66.3% saline v/v (i.e., 0.6% saline; 66.3% Saline control), and the balance being water or a mix of water and other components for a total of 100%. For controls and samples without SHMP: a Saline+Glycerol control contained 66.3% saline, 31.7% v/v glycerol, 0.17% ethanol, 1.93% water; samples contained 0.04% w/v EC16, 66.3% saline, 31.49% v/v glycerol, 0.17% v/v ethanol, 2% water. For samples with SHMP, the small volume of water was replaced with 10% w/v SHMP (0.2% final concentration) (Table 5). As a control for manipulation of cells, the media was replaced with fresh 100% MM.

TABLE 5
Properties of F100 formulation

| Mean ± SD | F100S | F100SD |
|---|---|---|
| pH | 4.76 ± 0.14 | 6.25 ± 0.04 |
| Particles/ml × 10⁹ | 2.67 ± 1.69 | 2.92 ± 1.14 |
| Median size (nm) | 142.0 ± 16.3 | 161.1 ± 21.3 |
| X10 (nm) | 65.0 ± 12.5 | 52.2 ± 15.0 |
| X90 (nm) | 391.2 ± 167.9 | 414.8 ± 52.2 |
| Zeta potential (mV) | −54.3 ± 23.3 | −43.0 ± 5.0 |
| Log titer reduction | 4.66 ± 0.00 | 5.35 ± 0.32 |
| Log titer reduction CV (%) | 0 | 6.0% |

Saline was found to be highly cytotoxic to MRC-5 cells, with the majority of the cells detaching from the plastic surface after just a few minutes exposure. Therefore, all cell treatments with Samples and controls were diluted 1:5 with MM (i.e., 80% MM final). Cells were exposed for 1 hr (equivalent to a viral infection procedure), and then the treatment was removed and replaced with 100% MM. After 24 hr recovery, cell viability was assessed by MTT staining, quantified by absorption spectroscopy at 540 nm.

### Time Response of Contact Inhibition Antiviral Activity of F18D

To determine the time response of contact inhibition antiviral activity of F18D EC16, a nasal formulation in saline containing 1.25 mM EC16 (n=3) with an OC43 viral titer of log 7.75-9.5 was incubated with virus at a 1:9 ratio (virus to formulation) for 1, 5, and 15 min before 10× serial dilutions and TCID50 assay.

### Transmission Electron Microscopy (TEM)

To acquire images of Coronovirus integrity with F18D or saline after 1 minute exposure, virus suspensions (either treated with F18D or saline for 1 minute) were fixed in 4% paraformaldehyde by adding an equal volume of 8% paraformaldehyde and 5 μl was transferred to a Formvar/Copper 200 mesh grid (Electron Microscopy Sciences, Hatfield, PA) and allowed to dry for 15 minutes. Excess solution was then removed using filter paper and virus was negatively stained by addition of 5 μl of 2% aqueous uranyl acetate.

To acquire images of Coronovirus integrity with F18D or saline after 30 minute exposure, virus suspensions (either treated with F18D or saline for 30 minutes) were fixed in 4% paraformaldehyde and 4% glutaldehyde at 1:9 ratio viral suspention:fixitive; and 5 μl was transferred to a Formvar/Copper 200 mesh grid (Electron Microscopy Sciences, Hatfield, PA) and allowed to dry for 15 minutes. Excess solution was then removed using filter paper and virus was negatively stained by addition of 5 μl of 2% aqueous uranyl acetate. Images were acquired in a JEM 1400 Flash Transmission Electron Microscope (JEOL, Peabody, MA) at 120 kV, using a Gatan One View Digital Camera (Gatan Inc., Pleasanton, CA).

### Statistical Analysis

The primary statistical tests were parametric one-way ANOVA (n=6 animals/group) with appropriate adjunct tests for homogeneity of variance, etc. and Fisher's exact test. Alpha was 0.05. GraphPad Prism version 6.x software (www.graphpad.com) was used for most analyses. Reported errors are given as standard deviation (SD) unless otherwise noted.

### Results

The study aims to develop intranasally applied green tea catechin derivative-containing formulations (such as in the form of a spray and/or irrigation) for using in any one of the methods disclosed herein (such as preventing and/or treating respiratory viral infection and post-infection symptoms, for example, minimizing Long COVID symptoms either through prevention or therapeutic approaches). The data described herein demonstrate that EC16 in nasal formulations effectively inhibit human coronavirus in vitro.

### Formulation F18-Containing EC16

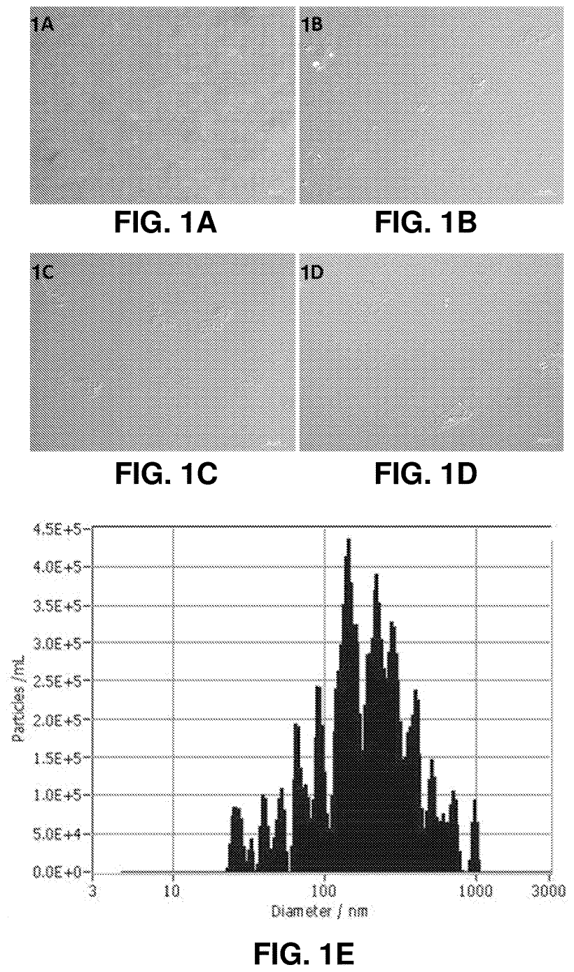

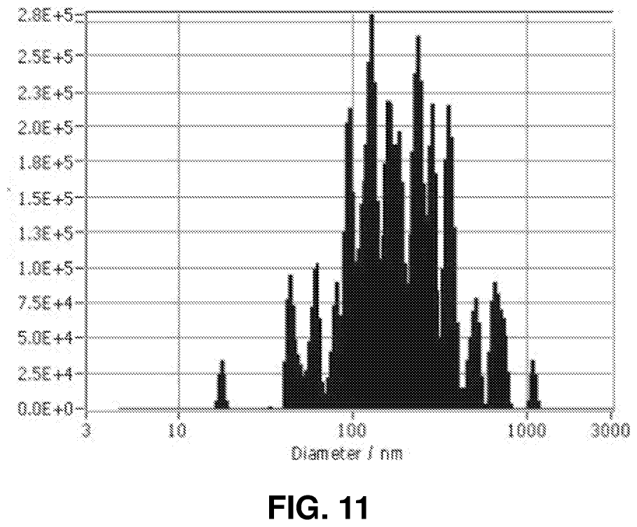

Dilution of F18 into aqueous buffers resulted in rapid formation of a flocculate precipitate that rapidly formed a cream. Microscopic images of the vortexed samples revealed large aggregates of material (FIGS. 1B-1D) together with barely visible particles, compared to image of the stock sample (FIG. 1A). Separation of the larger cream particles by centrifugation and quantitation of the polyphenol content showed that 10.8±10.6% of the polyphenol was recovered in the liquid phase, and 9.0±8.1% in the cream phase. Given that there was remaining liquid containing polyphenol in the cream phase, essentially all the polyphenol was present in the liquid suspension, and little (if any) in the bulk cream aggregate material. The composition of the cream was not further evaluated, and vortexed suspensions (i.e., including cream material) were used for subsequent experiments. After a short low-speed centrifugation to remove larger particles and aggregates (cream), the particle size distribution on 10-fold dilutions of F18 in saline was evaluated by nanoparticle tracking analysis (NTA). The particles showed high polydispersity, with sizes ranging from <100 nm to about 1 μm (FIG. 1E). The median size (average of 3 independent preparations) was 210±42 nm (SD), and the cutoff size for 90% of the particles was <547±190 nm (n=3). The mean initial particle density was 3.07±0.06×10⁷/ml. As shown in FIG. 11, there is a slight difference in particle size distribution of F18D compared to F18 (FIGS. 1A-1D). The median size was 186.6±20.62 nm (SD, n=3), and the cutoff size for 90% of the particles was <491±92 nm (n=3). The mean initial particle density was 4.57×10⁷/ml. The particles appeared to be more evenly distributed in F18D with about 50% more particles/ml than F18 (45.7 million vs. 30.7 million/ml). A particle size around 200 nm would be suitable for nasal drug delivery.

### Dose Effect Results

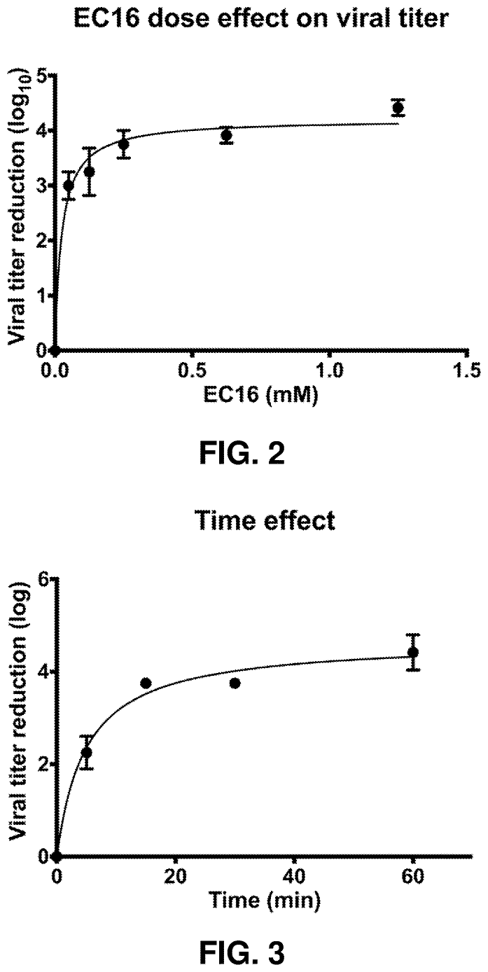

Incubation of virus with dilutions of F18 demonstrated a dose-dependent increase in antiviral activity of the formulation, as represented by an increase in the log₁₀ reduction in viral titer (FIG. 2). A plateau in viral titer reduction was evident at higher doses. Non-linear regression with a fit to a hyperbola gave a good fit to the data (r2=0.97, D'Agostino and Pearson omnibus K2 test of residual normality p=0.76). The Bmax (maximum specific binding) was 4.21±0.12 for the plateau in log₁₀ viral titer reduction, equivalent to a 99.994% reduction. The Kd (equivalent to the concentration giving a reduction of 50% of Bmax) was 0.025±0.005 mM. Using these values, the concentration of EC16 giving a reduction in titer 90% of Bmax (99.984% reduction) was 0.225 mM.

### Time Effect Results

To determine the time effect of EC16, the F18 stock was diluted to 1.25 mM with serum-free EMEM and incubated with OC43 virus (titer log 9.0/ml in serum-free EMEM) for different time periods before neutralization by immediate dilution with serum-free EMEM. FIG. 3 shows the results from three independent tests. The antiviral activity showed a rapid increase in log₁₀ titer reduction up to a plateau. Non-linear regression with a fit to a hyperbola gave a good fit to the data (r2=0.97, D'Agostino and Pearson omnibus K2 test of residual normality p=0.31). The Bmax was 4.77±0.23 for the plateau in log₁₀ viral titer reduction, equivalent to a 99.998% reduction, and consistent with the fold reduction observed in the dose response testing.

The Kd (equivalent to the time giving a reduction of 50% of Bmax) was 5.86±1.26 min. Using these values, a 90% Bmax reduction in titer was predicted at 42.9 min. The 30 min exposure used for dose testing was predicted to give a 3.994 log₁₀ reduction in titer, or 83.7% Bmax, and consistent with the values observed for dose testing.

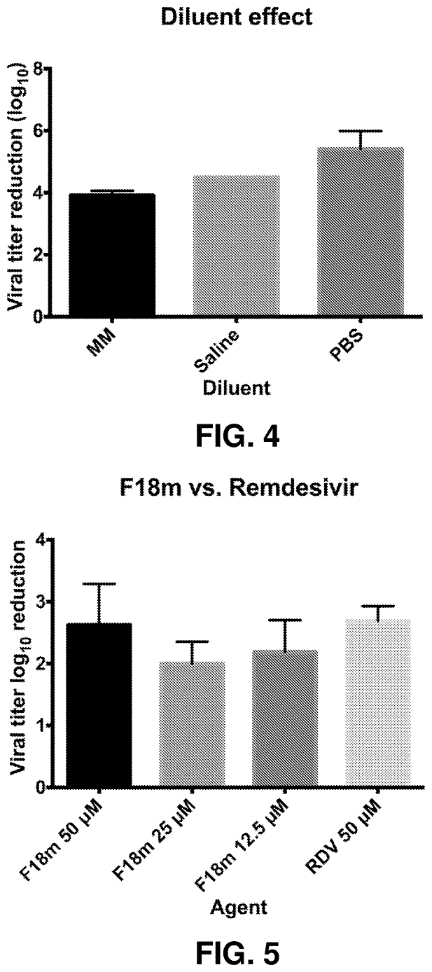

The effect of the formulation diluent on direct contact log₁₀ reduction was tested using a 30 min exposure of OC43 (titer log 9.75/ml) to 1.25 mM diluted formulation. As shown in FIG. 4, there was a difference between the three diluents tested (saline, PBS, and MM) (one-way ANOVA, (F2,6)=14.53; p=0.005). Post hoc testing (Tukey's multiple comparisons test) showed no significant difference between MM and saline (p=0.18), while PBS (mean log₁₀ 5.42±0.58) gave a higher reduction (p<0.04) in comparison to MM or saline (mean log₁₀ 3.92±0.14 and 4.50±0.00 respectively). That is, F18 diluted in PBS gave a 99.9996% reduction in viral titer.

### Direct Contact Test of F18m-Containing EC16m

The F18m formulation was diluted with serum-free EMEM to a working concentration of 1.40 mM EC16m. This formulation was incubated with virus (titer log 7.5-7.75/ml) for 30 min and then 10× serial dilutions were prepared and viral titer was determined. A 30 min incubation with 1.40 mM EC16m in serum-free EMEM resulted in a log₁₀ 3.00±0.43 (99.9%) reduction of viral infectivity (n=3). The reduction in titer with F18m at this dose was less than that seen with 1.25 mM EC16 dose tested with F18 (t-test, assumption of equal variance, p=0.040), likely due to the low titer in test.

### Tests of F18D and F18Dm

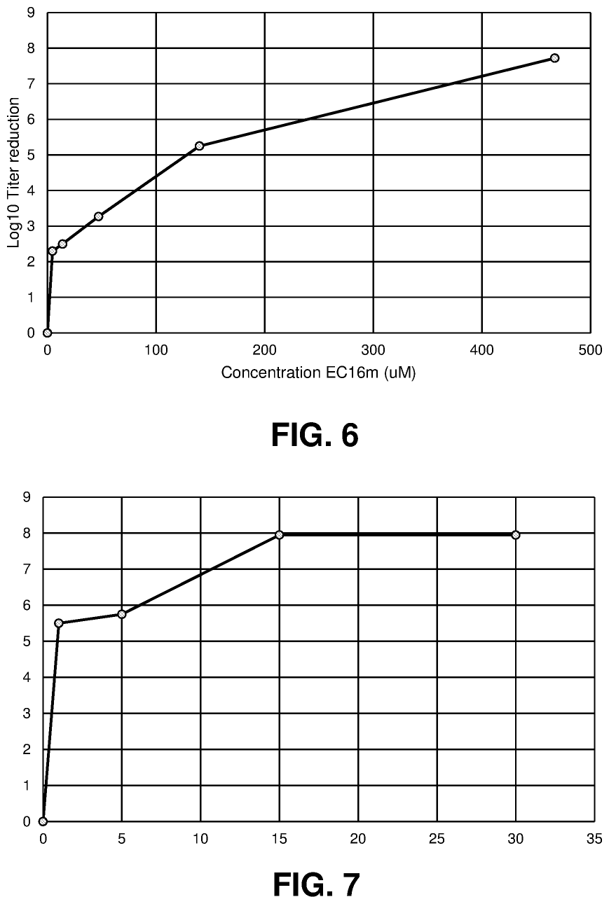

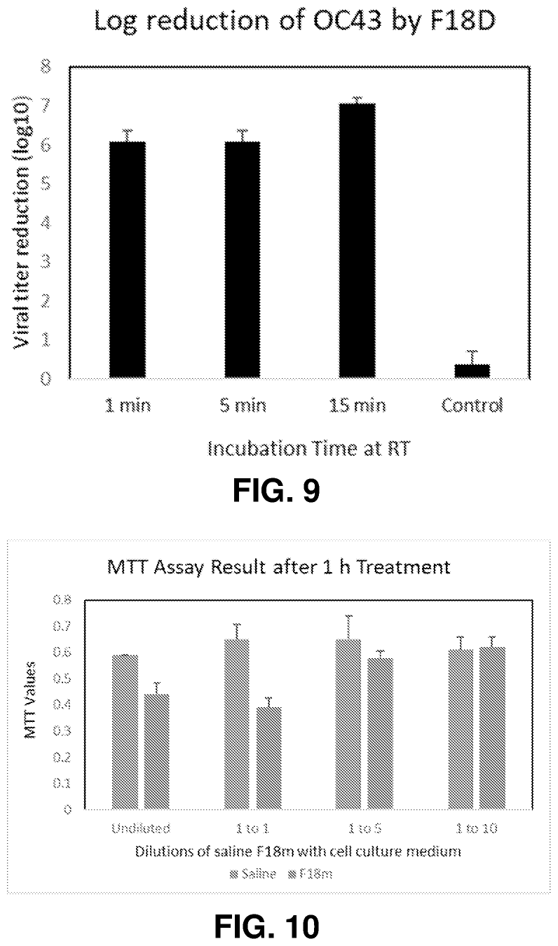

Results from the dose-dependent (FIG. 6) test of F18Dm, time-dependent (FIG. 7) test of F18Dm, and time-dependent (FIG. 9) test of F18D showed a higher efficacy of F18Dm and F18D compared to F18. In particular, as shown in FIG. 9, a 1-min incubation with F18D surprisingly reduced OC43 infectivity by log 6.08±0.29. If the formulation was incubated with the virus for 15 min, the reduction would be log 7.08±0.14 (n=3). In comparison, the control, which is the formulation without EC16 incubated with virus for 30 min, barely showed any reduction of OC43 infectivity (log 0.375±0.323).

### Pre-Infection Tests

To mimic one nasal application, the F18m formulation at 50 UM was added to the testing wells with a monolayer of cells for just 10 min before removal and exposure of the cells to virus (note: these tests used a lower viral concentration of logo 6.25/ml). The result indicated that 10 min pre-infection incubation led to a log 1.81±0.38 (98.45%) reduction in viral infectivity (n=4).

### Post-Infection Tests

To determine the post-infection dose effect of EC16m in serum-free EMEM, the F18m was diluted with serum-free EMEM to 50, 25, and 12.5 μM, and antiviral activity compared to 50 μM of Remdesivir as positive control, using a 10 min exposure to the agents before changing to media. There was no significant difference in the reduction in viral titer (one-way repeated measures ANOVA, Geisser-Greenhouse epsilon 0.506; F (1.519,4.557)=2.150; p=0.22 between treatment groups; n=4). A dose of just 12.5 μM F18m gave a log₁₀ titer reduction of 2.19±0.52, and 25 μM F18m gave a log₁₀ titer reduction of 2.00±0.36 (99.0%), about the same as 50% of the Bmax determined for this dose of F18. A 50 μM dose gave a log₁₀ 2.63±0.66 (99.77%) reduction, while exposure of infected cells to 50 μM remdesivir for the same time gave a viral titer log₁₀ reduction of 2.69±0.24 (99.80%).

### Time Responses Contact Inhibition Tests of the F18D EC16

The antiviral activity of the F18D EC16 nasal nanoformulation diluted with normal saline to a concentration of 1.25 mM EC16 was determined. The antiviral activity was calculated and expressed as log₁₀ reduction+standard deviation from three independent experiments and the control is the F18D formulation without EC16 incubated with virus for 30 min. A 1-min incubation reduced OC43 infectivity by log 6.08±0.29, which is equal to 5-min incubation. If the formulation incubated with the virus for 15 min, the reduction became log 7.08±0.14 (n=3). The control formulation (without EC16 nanoparticles) failed to significantly alter the infectivity (log 0.375±0.323). Direct contact incubation for 1 min and 5 min both reduced viral infectivity by 6.08±0.29 log₁₀ (n=3), while 15 min incubation led to a 7.08±0.14 log₁₀ reduction (n=3). Separately, F18D reduced infectivity of 229E α-coronavirus by 4.00±0.87 log after 30 min contact with a lower viral initial titer of 5.5 log.

### Antiviral Activity Tests of F18m

TCID50 assays of F18m were conducted to determine antiviral activity, the F18m diluted 25× (50 μM of EC16m) reduced OC43 viral replication by >100 fold by a single 10-minute post-infection application. EVOS images taken after 7-day post-infection of HNpEC by OC43 virus (FIGS. 15A-15F). The upper row are viral titer wells infected by 10⁻⁶, 10⁻⁷, 10⁻⁸ of virus without intervention. At 10⁻⁶, 10⁻⁷ concentrations, the cells were infected and died. At 10⁻⁸ dilution, the cells showed stress in morphology. In contrast, no cell infection was observed in the lower row, which was treated to 50 μM of EC16m in F18m formulation. This equals to a >2 log₁₀ reduction of viral replication in the cells. The properties and antiviral activity of F18 formulations in saline containing deflocculants at 1% are described in Table 4.

TABLE 4
Properties and antiviral activity of F18 formulations in saline containing deflocculants at 1%

| | | | | F18 + PA | F18 + PA | |
|---|---|---|---|---|---|---|
| | F18 | | | (pH 8.18 | (pH 6.82 | |
| Mean ± SD | (Control) | F18 + PVP | F18 + PVA | stock) | stock) | F18 + SHMP |
| pH | 5.29 ± 0.40 | 4.12 ± 0.71 | 5.31 ± 0.04 | 7.65 ± 0.27 | 7.19 ± 0.03 | 6.44 ± 0.07 |
| Particles/ml | 3.80 ± 2.45^{a} | 72.8 ± 15.2^{b} | 82.5 ± 4.4 ^{c} | 33.1 ± 35.0 | 50.0 ± 16.0 | 17.1 ± 23.0 |
| x10⁸ | | | | | | |
| Median size | 140.5 ± 16.6 | 144.7 ± 6.6 | 144.2 ± 17.7 | 154.4 ± 44.2 | 123.8 ± 15.0 | 132.9 ± 30.7 |
| (nm) | | | | | | |
| X10 (nm) | 57.5 ± 15.3 | 54.3 ± 7.8 | 53.1 ± 3.9 | 57.8 ± 21.3 | 55.7 ± 12.8 | 60.7 ± 16.0 |
| X90 (nm) | 272.8 ± 34.6 | 327.6 ± 62.1 | 316.0 ± 53.0 | 358.8 ± 148.4 | 216.4 ± 20.7 | 266.7 ± 53.5 |
| Zcta potential | −48.5 ± 0.8 | −30.5 ± 14.5^{d} | −19.2 ± 0.4^{e} | −41.9 ± 11.4 | −41.1 ± 8.5 | −52.6 ± 10.9 |
| (mV) | | | | | | |
| Log titer | 2.40 ± 1.04 | 0.86 ± 1.09 | 1.7 ± 0.29 | 4.38 ± 0.38 | 2.05 | 5.70 ± 0.91 |
| reduction | | | | | | |
| Log titer | 43.3 | 127 | 17.1 | 8.7 | | 16.0 |
| reduction CV | | | | | | |
| (%) | | | | | | |

mean and SD determined from n = 5 replicate experiments, except for F18 + PA (n = 6).
^{a}Significantly lower than F18 + PVP, F18 + PVA, and F18 + PA pH 6.82 (p ≤ 0.015)
^{b}Significantly higher than F18 + PA pH 8.18, F18 + SHMP (p = 0.03 and 0.02 respectively)
^{c} Significantly higher than F18 + PA pH 8.18 and F18 + SHMP (p = 0.007 and 0.005 respectively)
^{d}Significantly less negative than F18 + SHMP (p = 0.001)
^{e}Significantly less negative than F18, F18 + PA pH 8.18, pH 6.82, or F18 + SHMP (p ≤ 0.016)

### Cytotoxicity Study of F18m in Normal Saline

To determine the cytotoxicity of F18m with normal saline, the primary HNPEC, PromoCells were allowed to form a monolayer, followed by incubation with F18m and normal saline for 60 min (FIG. 10). The solutions were replaced with cell culture medium. The MTT assay was performed the next day using CytoSelect MTT Cell Proliferation Assay kit (Cell Biolabs, Inc). The 1.4 mM F18m in saline (undiluted) and a 1:1 dilution (0.7 mM EC16m) resulted in reduction in MTT values, while 1:5 and 1:10 dilutions were not statistically different from saline dilutions (one-way ANOVA, p>0.06, p>0.87, respectively, n=3). These results show that at 1.40 and 0.70 mM, EC16m nanoparticles (3 million and 1.5 million nanoparticles on 10,000 cells, according to particle density estimates in FIGS. 1A-1D) induced cytotoxicity in HNPEC. When the particle number was lowered to 0.6 million (1:5, 280 μM) and 0.3 million particles/10,000 cells (1:10, 140 μM), the MTT values had no statistical difference from saline dilutions, which show that an EC16m concentration lower than 0.02% (280 μM) is not toxic to HNEpC (90 nanoparticles/cell).

### Cytoxicity Study of F18BC and F18DC in Normal Saline

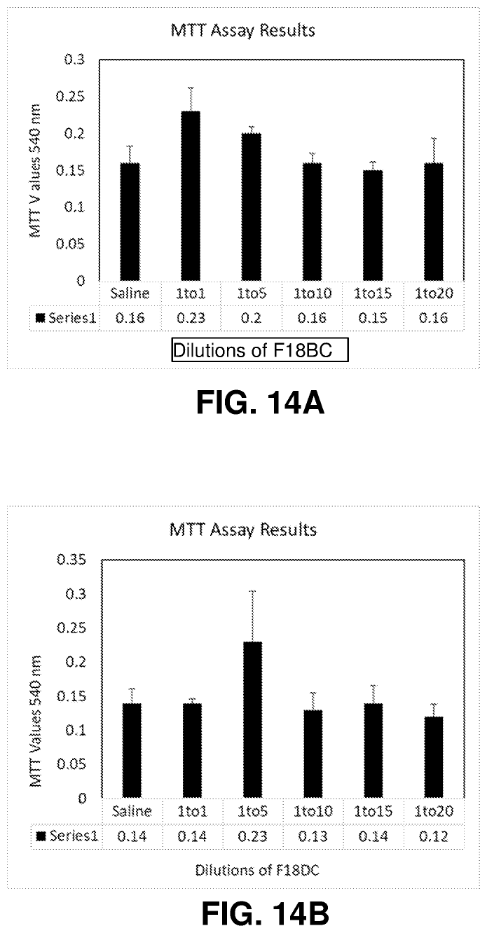

The MTT cell viability assay was conducted to determine the cytotoxicity of F18BC and F18DC. The MTT cell viability assay demonstrated that both formulations showed comparable cell viability with saline-treated cells for 60 minutes. When the F18BC was diluted 2 or 5 times with basal medium, or when the F18DC was diluted 5 times, the cell viability was higher than saline-treated cells (FIGS. 14A and 14B).

### Cytotoxic Studies of F18m and F18Dm in HCT-8 Cells

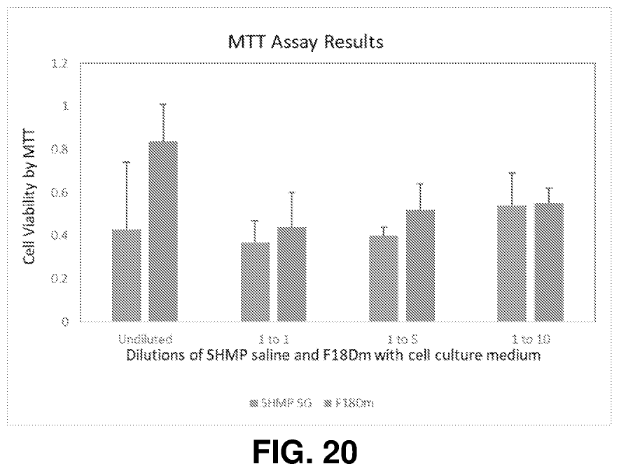

Cytotoxic studies of F18m in HCT-8 cells discovered that undiluted F18 has higher cell viability than undiluted saline, as well as medium diluted F18 1:1 (0.05% EC16m) (FIG. 19). Formulations diluted 1 to 5 and 1 to 10 have similar cell viability levels. Cytotoxic studies of F18Dm in HCT-8 cells discovered that undiluted F18D has higher cell viability than undiluted SHMP/saline/glycerol (FIG. 20). Formulations diluted 1 to 1, 1 to 5, and 1 to 10 have similar cell viability levels.

### Cytotoxicity Studies of F18/F100 Formulations for HCT Cells and MRC-5 Cells

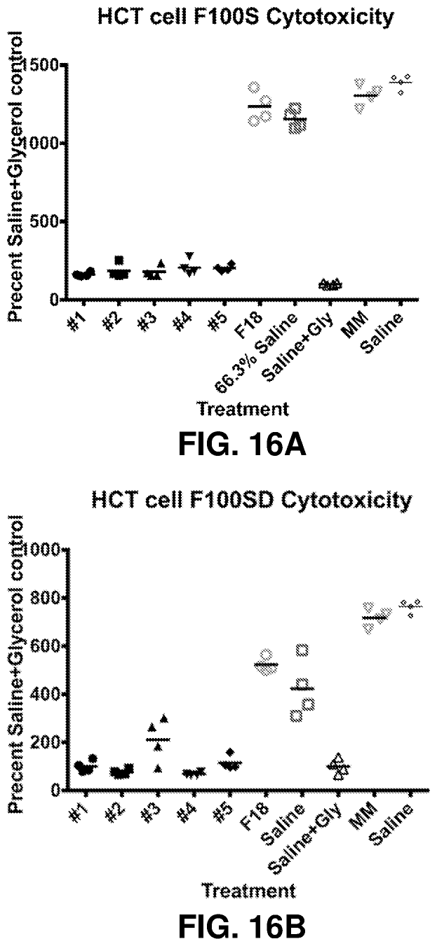

To test for cytotoxicity in HCT cells, the three controls (67% saline, saline+glycerol, and MM) showed a highly significant difference between groups (one-way ANOVA, p<0.0001) (FIGS. 16A and 16B). Tukey's multiple comparisons test showed significant differences between all three pairwise comparisons (p≤0.007). With MM Control set to 100% MTT staining, treatment of cells with the Saline control (0.119% w/v NaCl, 80% v/v MM) reduced staining to 88.5% (p=0.007), while increasing the saline concentration to 0.18% NaCl in the control increased staining to 106%, not significantly different from MM (p=0.14).

Treatment with the Saline+Glycerol control (0.119% NaCl, 6.3% v/v glycerol, 80% v/v MM) reduced staining to 7.7% of MM control (p<0.0001). The Saline+Glycerol control was also significantly less than the Saline control (p<0.0001). Therefore, glycerol (and a trace of ethanol) in this mixture was highly cytotoxic, as measured by MTT staining. Consistent with this, treatment with the F18 control (containing 0.008% w/v EC16, 0.72% v/v glycerol, 0.072% ethanol, and 0.144% w/v NaCl) gave 94.7% of the MM staining (p=0.49). Importantly, examination of the control group cells by microscopy revealed no evident effect in comparison to the MM control. Therefore, the large reduction in MTT staining with the Saline+glycerol control did not represent death and loss of cells. These results suggested that the HCT cells were highly sensitive to the osmotic strength of the treatments, and responded by reducing the metabolic activity responsible for MTT staining, and this response was still present 24 hr after treatment.

Individual MTT OD values for the five batches of F100S tested, and the controls, were normalized to the mean Saline+Glycerol control value and compared to this control (100%) by one-way ANOVA (Dunnett's multiple comparisons test). All five F100S samples tested gave a value greater than 100% (162-206%). All but the largest of the five values showed no significant difference to the Saline+Glycerol control (p≥0.053), while the largest was significantly larger (p=0.043). However, the five F100S samples were not significantly different from each other (p=0.44).

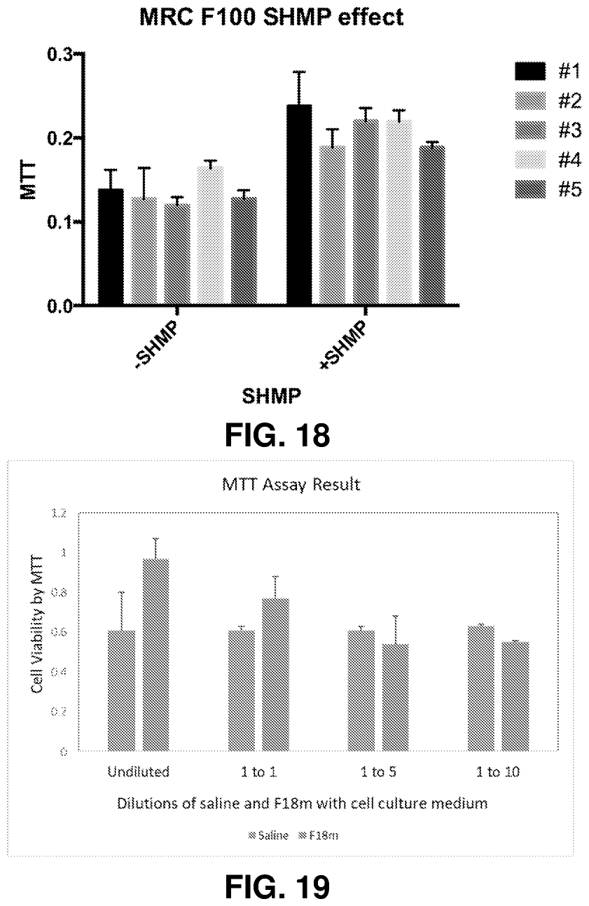

When 0.2% SHMP was included in the controls, the MTT value for the Saline+Glycerol control was not changed significantly (p=0.63; two-way ANOVA, Sidak's multiple comparisons test) (FIG. 18). However, the values for both F18 and 67% saline were decreased significantly (to 76.8% and 66.6% respectively; p=0.004 and 0.002). With or without SHMP, the values for F18 and 66.3% saline were not significantly different from each other (p=0.076 and 0.64 respectively). However, with or without SHMP, both were significantly higher than the corresponding Saline+Glycerol control (p<0.0001). Thus, 0.2% SHMP increased the reduction in MTT staining caused by 66.3% saline of by F18, but had no further effect on the reduction caused by 66.3% Saline+Glycerol.

For the five F100 samples, SHMP had no significant effect on the MTT value (p=0.32; two-way ANOVA). However, there was a significant difference between the samples (p=0.003), and a significant interaction effect (p=0.001), with sample #3 showing a significantly higher MTT value in the presence of 0.2% SHMP (p=0.0004), whilst the other four samples did not show this effect (p≥0.33). In comparison to the Saline+Glycerol control containing SHMP, Sample #3 gave a significantly higher value (p=0.04; 211%), but the other four samples were not significantly different (68-115%; p≥0.96, one-way ANOVA, Dunnett's multiple comparisons test). These results show that EC16 in the F100 formulation did not have a cytotoxic effect on HCT cells, as measured by MTT, beyond the effect of the excipient formulation.

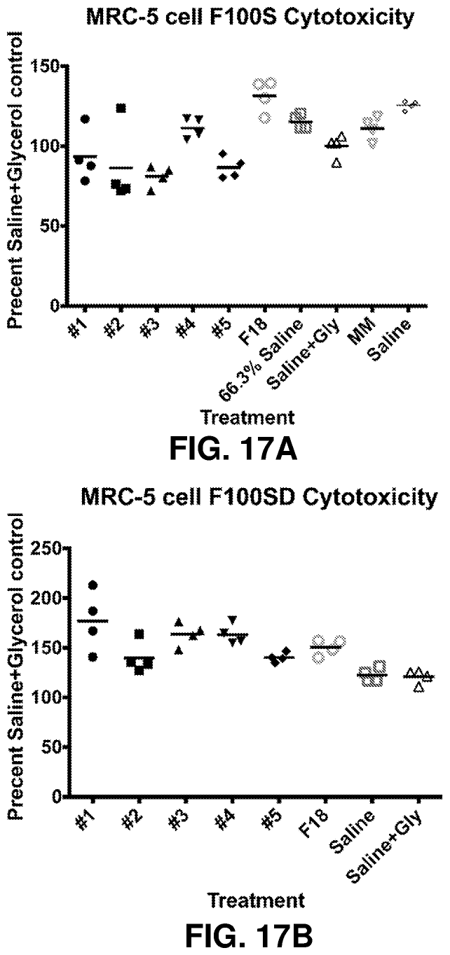

For MRC-5 cells, the three controls (66.3% Saline, Saline+Glycerol, and MM) showed a significant difference between groups (one-way ANOVA, p=0.02) (FIGS. 17A and 17B). With MM Control set to 100% MTT staining, treatment of cells with the Saline control (0.119% w/v NaCl, 80% v/v MM), or the Saline+Glycerol control (0.119% NaCl, 6.3% v/v glycerol, 80% v/v MM) gave values that were not significantly different (104 and 90.0% respectively; p=0.63 and 0.086; one-way ANOVA, Tukey's multiple comparisons test). However, the Saline+Glycerol MTT value was significantly lower than 66.3% Saline (p=0.02). Increasing the saline concentration to 0.18% NaCl in the control increased staining to 113%, not significantly different from MM (p=0.06) or from 66.3% saline (p=0.27).

Therefore, for MRC-5 cells, glycerol (and a trace of ethanol) in the excipient mixture had a very modest cytotoxic effect, as measured by MTT staining, and the saline concentration appeared to have no effect. Consistent with these observations, examination of the control group cells by microscopy revealed no evident effect in comparison to the MM control.

These results show that the MRC-5 cells were not markedly sensitive to the osmotic strength of the treatments, as detected by MTT staining 24 hr after treatment. Individual MTT OD values for the five batches of F100S tested, and the controls, were normalized to the mean Saline+Glycerol control value (i.e., the excipient) and compared to this control (100%) by one-way ANOVA (Dunnett's multiple comparisons test). The five F100S samples tested gave a value ranging from 81.1-111% of the Saline+Glycerol control, and none were significantly different from the control (p≥0.14), and the five F100S samples were not significantly different from each other (p=0.074). Treatment with the F18 control (containing 0.008% w/v EC16, 0.72% v/v glycerol, 0.072% ethanol, and 0.144% w/v NaCl) gave 118% of the MM staining, which was significantly higher (p=0.006). Comparison of the F100S samples to MM showed that #2, 3 and #5 were significantly lower (p≤0.03; 86.4, 81.1, 86.6% respectively), while #1 and #4 (93.5 and 111%; p≥0.19) were not. Collectively, the five samples showed consistent results, and as measured by MTT there was no evidence for a cytotoxic effect of EC16 in the F100 formulation relative to the excipient, and only a modest effect relative to MM.

When 0.2% SHMP was included in the controls, the MTT values for the F18, 66.3% Saline, Saline+Glycerol control were not changed significantly (p=0.16; two-way ANOVA, Sidak's multiple comparisons test). There was a significant difference between the groups (p≤0.0001), with no interaction effect (p=0.17). In the absence of SHMP, F18 was significantly higher than 66.3% saline (114%; p=0.0002) and Saline+Glycerol (132%; p=0.0001), indicating a stimulation of cellular activity by F18 relative to the controls, but with no further increase due to SHMP.

For the five F100 samples, SHMP had a significant effect on the MTT value (p<0.0001; two-way ANOVA), with all five samples showing a fold-increase from 1.34-1.84; that is, SHMP stimulated MTT staining in F100. There was a significant difference between the samples (p=0.008), but no significant interaction effect (p=0.11). In the absence of SHMP, there was no significant difference between the samples, but in the presence of 0.2% SHMP, Sample #1 was significantly higher than samples 2 and 5 (1.08-fold each; p≤0.036).

In comparison to the Saline+Glycerol control containing SHMP, Samples #1, 3 and 4 were significantly higher (1.35-1.46-fold p≤0.002, one-way ANOVA, Dunnett's multiple comparisons test), while Samples 2 and 5 were not (both 1.16-fold; p≥0.28).

Broadly these results show that EC16 in the F100 formulation did not have a cytotoxic effect, as measured by MTT, beyond the modest effect of the excipient formulation. Sample formulations with SHMP uniformly showed an increase in MTT value compared to without, but not all samples with SHMP showed a significant increase relative to the Saline+Glycerol control.

### TEM Results

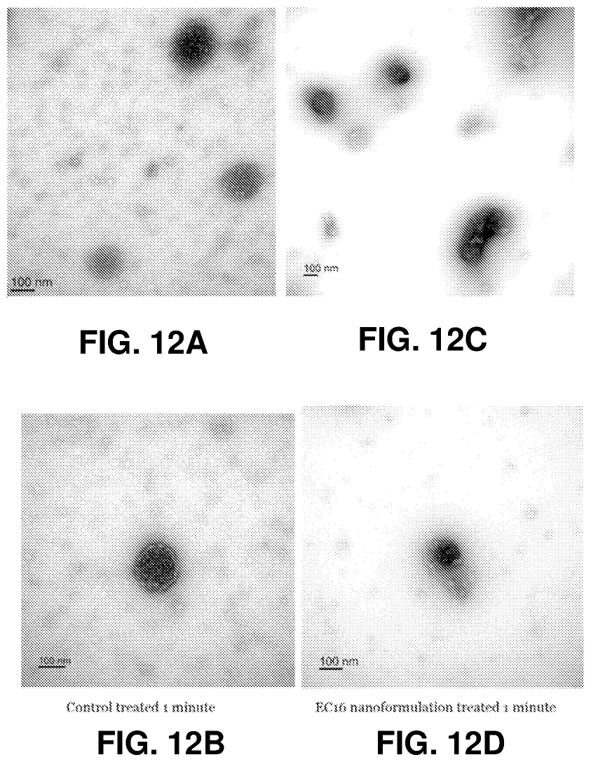

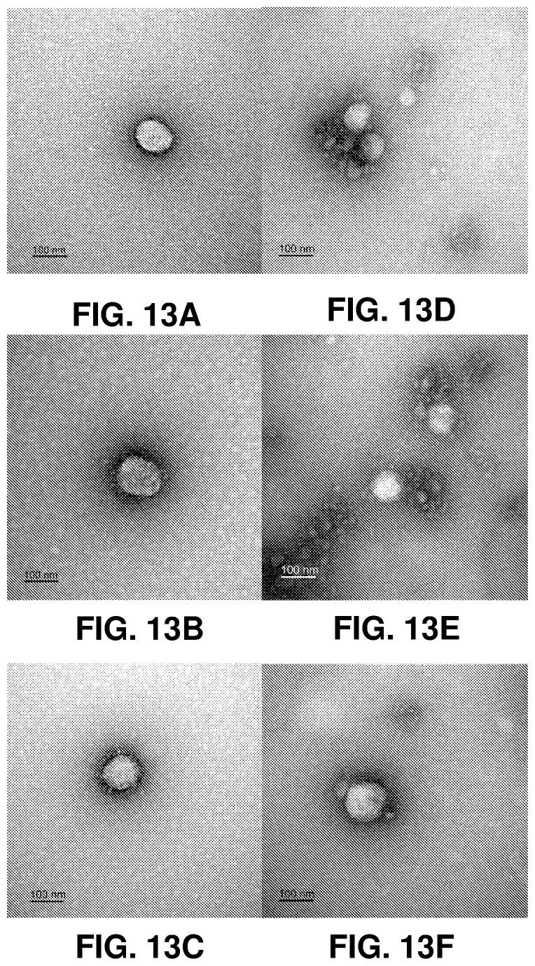

Images of human β-coronavirus OC43 after one and thirty minute exposures with F18D or saline were collected with in a JEM 1400 Flash Transmission Electron Microscope. Human β-coronavirus OC43 lost structure integrity after 1-min contact with F18D EC16 nasal nanoformulation (contains food grade material dispersed in normal saline) (FIGS. 12A-12D). Human β-coronavirus OC43 completely lost typical virus structure morphology after 30-min contact with F18D EC16 nasal nanoformulation (FIGS. 13A-13F). These TEM results validated the results of antiviral tests (TCID50 assay) of 1 min contact with OC43 virus shown in FIG. 9. The control has no activity on the virus, and F18D EC16 nanoformulation killed >99.9999% of virus.

### Discussion

The current study tested the feasibility of using EC16 as a nasal delivered drug to provide antiviral activity in terms of contact inactivation and pre- and post-infection inactivation of viral infectivity. The human nasal cavity is made of the respiratory epithelium (RE), which contains ciliated cells, basal cells, brush cells, and secretory cells; and the olfactory epithelium (OE), which contains olfactory sensory neurons, sustentacular cells, microvillar cells, globose basal cells, and horizontal basal cells. The non-neuronal cells express ACE2 and TMPRSS2, and the olfactory sensory neurons express neuropilin-1, which facilitate SARS-CoV-2 infection. Entry of SARS-CoV-2 into the nasal cavity results in infection and initial replication in the RE during the early stage of COVID-19, mainly in the ciliated cells that are rich in ACE2 and TMPRSS2. The rapid accumulation of SARS-CoV-2 in RE could cause concomitant infection in the OE. Indeed, recent clinical and animal studies show that SARS-CoV-2 infection of the olfactory sensory neurons and their support cells in the OE results in local inflammation and apoptosis, which could be the mechanisms leading to OE destruction, anosmia, and other neuronal dysfunctions in the CNS. Thus, active SARS-CoV-2 replication in RE, OE, and the olfactory bulb may be the cause of acute anosmia. And persistent presence of the virus in the RE and OE cells could be associated with chronic neurologic symptoms. In addition, asymptomatic patients have a nasal viral load comparable to symptomatic patients, showing that both symptomatic and asymptomatic patients are at risk for anosmia. Therefore, inactivation and clearance of viral particles in the nasal cavity can effectively minimize the risks for post COVID neurologic symptoms. However, neutralizing antibodies, either injected in the nasal cavity or acquired via vaccination, are not effective to reduce the SARS-CoV-2 viral load in the nasal cavity due to robust viral replication in the nasal turbinate. Further, if the EC16 formulation not only perform the beneficial activity in the nasal epithelia, but also provide effects in the central nerve system (CNS), such as decreasing reactive oxygen species, it would be a first-in-class drug to prevent and minimize SARS-CoV-2 associated neurologic symptoms, including Long COVID.

The US FDA have approved a number of nasal delivered drugs to treat different symptoms, such as Spravato (esketamine) nasal spray for depression (fast-track), Astepro (azelastine hydrochloride nasal spray, 0.15%) for seasonal and perennial allergic rhinitis, Narcan (naloxone hydrochloride) nasal spray for opioid overdose, and Ryaltris (mometasone furoate monohydrate) nasal spray for seasonal allergic rhinitis, etc. However, there is no intranasally administered drug for use against respiratory viral infection or post-infection symptoms.

EC16 is a lipid-soluble compound derived from EGCG by esterification with palmitate. Since the bioavailability of EGCG is very low, nasal delivery could be a route to administrate lipid-soluble EC16 for neuroprotection. Compared to water-soluble EGCG, EC16 is significantly more potent against influenza virus, herpes simplex virus, and norovirus. Other advantages of EC16 vs. EGCG are that EC16 is more stable and long-lasting. In addition, the US FDA approved the use of tea polyphenol palmitates (contains 50% EC16) as a GRAS green tea extract (FDA GRAS Notice 772). EC16 undergoes hydrolysis after consumption to free EGCG after entering cells. These demonstrated that the use of EC16 as a nasal formulation meets the GRAS requirement. EC16 has the potential to protect nasal epithelial cells from SARS-CoV-2 infection as well as exert anti-inflammatory, antioxidant, and neuroprotective effects.

Previous studies showed that neuroinvasion of SARS-CoV-2 occurs closely following the peaking of viral replication in the nasal epithelia. Thus, the formulations must possess rapid antiviral actions to inactivate the coronavirus in the nasal epithelial cells and be able to deliver the multiple benefits of EC16 into the brain, bypassing the blood-brain barrier (BBB) and digestive system. The nasal delivery method would overcome the well-documented poor bioavailability of EGCG, which serum maximum concentration is at in the sub-micromolar range (0.57 μM), well below the effective concentration for beneficial effects. If EC16 is applied to the nasal epithelial cells, the long chain fatty acyl group would allow EC16 to attach to the cell membrane for prolonged effect against SARS-CoV-2 and its variants.

The (1) antiviral activity and (2) anti-inflammatory, antioxidant, and neuroprotection activities of exemplary nasal formulations containing EC16 in both nasal neuroepithelia and the brain were tested to demonstrate the feasibility of formulating candidate with antiviral properties for COVID and Long COVID use.

Due to the lipid-soluble nature of EC16, the solubility of EC16 is very low in aqueous solutions. In here, EC16 are in the form of particles ranging from nano to micro-meters in an aqueous suspension. Among 62 formulations tested, the F18 glycerol-based stock formulation showed the most antiviral activity. Dilution of F18 into aqueous buffer systems resulted in rapid formation of a flocculate suspension, with little, if any, polyphenol in the flocculate material, which contained large aggregates of various-sized particles (FIGS. 1B-1D). Analysis of particle size distribution in the saline liquid phase showed a broad polydisperse suspension ranging from about 40 nm to about 1000 nm (FIG. 1E).

The F18 formulation diluted in EMEM showed antiviral activity when virus was directly exposed to it, with a maximum inhibitory effect of log₁₀ 4.21±0.12 (SE) (99.996%) at saturating concentrations in the 1 mM range (FIG. 2). Based on a regression analysis and determination of the curve constants, a concentration of 0.225 mM can produce a log₁₀ 3.79 reduction (90% of maximum). Thus, experiments conducted with 1.25 mM were in the saturated region. The antiviral results of F18 diluted in serum-free EMEM show that at 50 μM (approximately 40 ng/ml), a 30 min incubation with the virus reduced the infectivity by 99.90%. A concentration of 8 μM can produce a 90% reduction (the EC90; log₁₀ 1 reduction). This is significantly more potent than the antiviral activity of EGCG against SARS-CoV-2 (one-hour incubation EC90=69 μM).

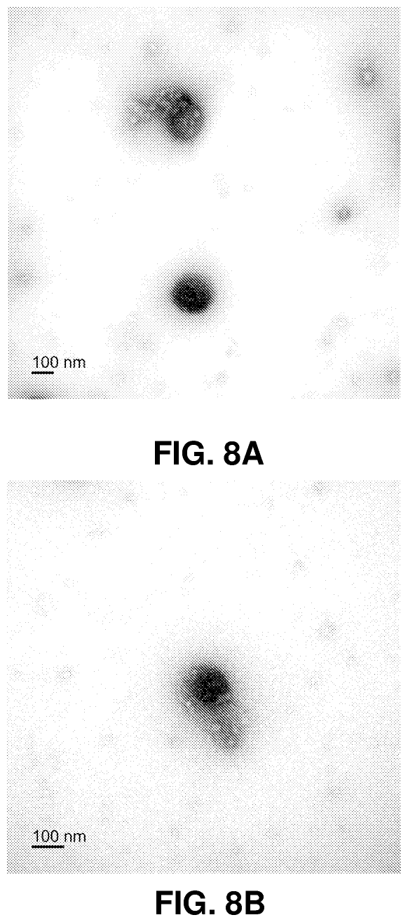

At a saturating concentration of 1.25 mM EC16, a 5-min direct contact with the virus reduced the viral infectivity by >99% (FIG. 2). These results show that the rapid and potent inactivation of viral infectivity may be associated with a different mechanism of action compared to antiviral drugs in use, such as the COVID drug Remdesivir (a nucleoside analog to inhibit RNA polymerization), which has no known contact inhibition of coronavirus. Further, as shown in FIGS. 8A and 8B, incubation of viruses with diluted F18 for 1-min resulted in virus deformation by binding to EC16.

For an ongoing animal study and a future human study, the F18 formulation is/will be tested by dilution in PBS and saline, which showed similar direct virus inhibition efficacy results (FIG. 3), although dilution in PBS gave greater reduction (9.3-fold). Without being bound to any theories, it may be that the phosphate content could enhance the antiviral activity of EC16, such as by modifying the surface charge of particles. Regardless, antiviral activity of F18 formulations tested herein is at a very high level (log 4.50 vs. log 5.42).

EC16 is a mixture of EGCG-palmitates, with the majority being EGCG-mono-palmitate (EC16m), followed by EGCG-di-palmitates, and EGCG-tri-palmitates. Therefore, the single molecule EC16m will most likely be the new drug form. A series of initial tests for EC16m was performed with the F18m formulation of EC16m in serum-free EMEM dilutions. After a 30 min incubation with 50 μM EC16m the infectivity of the OC43 virus was reduced by 99.9% (FIG. 5), similar to that seen with EC16 (99.996%, FIG. 2) at a higher dose. The measured log₁₀ reduction value is influenced by the titer of the virus used in the tests, with a lower titer resulting in a lower proportionate log₁₀ reduction. Since the F18m tests used a lower titer virus preparation (log 7.75 in comparison to >log 9.0 in EC16 contact inhibition tests), EC16m has similar activity to EC16.

A human study show that when symptoms appear during initial SARS-CoV-2 infection, the nasal cavity viral load is less than log₁₀ 8 and peaks (day 2 to 6) at >log 9. Therefore, the tests conducted herein were at clinically relevant viral loads. Pre-infection incubation of cells with 50 μM (approximately 35 ng/ml) EC16m gave a 98.45% inhibition of subsequent viral replication in the cells. On the other hand, without direct contact with the virus, a 10 min incubation of infected MRC-5 cells with 50 μM EC16m (approximately 35 ng/ml) reduced the viral replication by 99.77%, comparable to the reduction seen with remdesivir (FIG. 5). Even at just 12.5 μM, EC16m inhibited viral replication by >99% after 10 min post-infection treatment with the cells before removal. These initial results from a base EC16m formulation demonstrated that EC16m entered the cells and blocked the viral replication effectively, because the inhibition was the result of a single application and viral titer was observed over a 4- to 7-day incubation period. Repeated applications of EC16m may produce a higher inhibitory effect on viral replication.

Cytotoxic studies of the F18m formulations with EC16m demonstrated that viral replication reduction occurs below cytotoxic concentrations. Activity wise, 12.5 μM EC16m in F18m formulation reduced 99% viral replication with a single 10-min post infection application (FIG. 10, and cytotoxic studies describes no toxicity to HMEpC at concentrations lower than 0.02% (280 μM).

Live cells can convert MTT to an insoluble purple formazan dye that can be quantified by absorption spectrophotometry. Since the amount of formazan formed is proportional to the live cell number, MTT staining can provide a value proportional to number of live cells. In cytotoxicity assays, a reduction in the MTT value after treatment is taken as evidence of cytotoxicity (fewer cells being equivalent to cell death), and an increase being evidence of a stimulation of cell growth. Although not completely understood, it is believed that MTT conversion is mediated by components of the energy production system. This system is often modulated by agents, and therefore MTT staining alone no longer simply reflects cell number and growth or death. For example, with MRC-5 cells, inclusion of SHMP in the treatment increased MTT staining. SHMP contains energy rich phosphodiester bonds and is known to provide an energy source to cells. However, MTT staining can still provide an estimate of changes to cells caused by a treatment. Here, using two types of cells (MRC-5 and HCT), the F100 formulation (with or without SHMP) showed no consistent cytotoxicity that could be ascribed to EC16.

The results presented here demonstrated that F18 base formulations containing EC16 or EC16m have antiviral activities to either rapidly inactivate human coronavirus by direct contact or inhibit viral entry and replication without direct contact with the virus.

### Conclusion

For the first time, epigallocatechin-3-gallate-palmitate(s) in a formulation suitable for intranasal administration to minimize post-COVID neurologic symptoms through its strong and rapid antiviral and other beneficial properties. Ongoing studies include testing of the broad-spectrum of antiviral activity of EC16 on human α-coronavirus (229E) in human primary nasal epithelial cells, SARS-CoV-2 variants in vitro, and in K18-hACE2 mice. In addition, saline-based formulations with higher potency are being explored. For new drug development, the next phase studies will include formulation finalization (chemistry, compatibility, stability, validation, etc.), and initial efficacy and toxicity with in vivo and ex vivo models, in order to collect data for pre-Investigational New Drug (IND) studies (including chemistry-manufacturing-control, pharmacology-toxicity, etc.). The advantage of the formulation is that all ingredients are known nontoxic molecules, and saline is regularly used in nasal irrigation/spray solutions. In conclusion, a simple nasal formulation was developed to incorporate lipid-soluble EC16 in saline, without surfactant, to rapidly inactivate human β-coronavirus OC43 by contact or inhibit viral replication by a single 10-min application on infected cells. These results show that EC16 nasal applications are effective prophylactic and therapeutic methods to minimize respiratory virus associated, such as COVID and post-COVID symptoms.

The full names of certain abbreviations as used herein is provided in the following table:

| Abbreviation | Full Name |
|---|---|
| PS80 | polysorbate 80 |
| DMSO | dimethyl sulfoxide |
| PA | sodium polyacrylate |
| PVP | polyvinyl pyrrolidone |
| PVA | polyvinyl alcohol |
| CMC | sodium carboxymethyl cellulose |
| SHMP | sodium hexametaphosphate |

### REFERENCES

1. Groff, et al. JAMA Netw Open 2021, 4, e2128568.
2. Kumari, et al. Viruses 2021, 13.
3. Sungnak, et al. Nat Med 2020, 26, 681-687.
4. Gallo, et al. Mucosal Immunol 2021, 14, 305-316.
5. Wu, et al. Cell 2023, 186, 112-130 e120.
6. Dinda, et al. Phytomed Plus 2023, 3, 100402.
7. Goncalves, et al. Biomolecules 2021, 11.
8. Hsu, Inflamm Allergy Drug Targets 2015, 14, 13-18.
9. Kaihatsu, et al. Molecules 2018, 23.
10. Mokra, et al. Int J Mol Sci 2022, 24.
11. Payne, et al. Biomolecules 2022, 12.
12. de Oliveira, et al. Food Chem Toxicol 2013, 52, 207-215.
13. Dickinson, et al. Microbiology & Infectious Diseases 2018.
14. Zhao, et al. Oral Surg Oral Med Oral Pathol Oral Radiol 2015, 120, 717-724.
15. Zhong, et al. Microbiol Infect Dis 2021, 5.
16. Hurst, et al. Microbiol Infect Dis 2021, 5.
17. 772, U.G.N. GRAS Notice for Oil-Soluble Green Tea Extract (Green Tea Catechin Palmitate). 2018.
18. de Melo, et al. Sci Transl Med 2021, 13.
19. Ahn, et al. J Clin Invest 2021, 131.
20. Bryche, et al. Brain Behav Immun 2020, 89, 579-586.
21. Zhou, et al. Cell Host Microbe 2021, 29, 551-563 e555.
22. Ra, et al. Thorax 2021, 76, 61-63.
23. Giunchedi, et al. Pharmaceutics 2020, 12.
24. Khan, et al. J Control Release 2018, 291, 37-64.
25. Maaz, et al. Pharmaceutics 2021, 13.
26. Bonferoni, et al. Pharmaceutics 2020, 12.
27. de la Torre, et al. Clin Nutr 2020, 39, 378-387.
28. Singh, et al. Nutr J 2016, 15, 60.
29. Chen, et al. Handbook of Green Tea and Health Research, Mckinley, H., Jamieson, M., Eds.; Nova Science Publishers, Inc.: New York, 2009; pp. 45-61.
30. Hsu, et al. Household and Personal Care TODAY 2009, n2, 33-36.
31. Mori, et al. Bioorg Med Chem Lett 2008, 18, 4249-4252.
32. Cai, et al. Molecules 2018, 23.
33. Yang, et al. Cancer Epidemiol Biomarkers Prev 1998, 7, 351-354.
34. Savela, et al. J Clin Microbiol 2022, 60, e0178521.
35. Baxter, et al. Ear Nose Throat J 2022, 1455613221123737.
36. Huijghebaert, et al. Eur J Clin Pharmacol 2021, 77, 1275-1293.
37. Panta, et al. Explore (NY) 2021, 17, 127-129.
38. Xu, et al. Molecules 2017, 22.
39. Everette, et al. J Agric Food Chem 2010, 58, 8139-8144.
40. Helwa, et al. PLOS One 2017, 12, e0170628.
41. Reed, et al. American Journal of Epidemiology 1938, 27, 493-497.
42. Amish, et al. JAMA Otolaryngol Head Neck Surg. Published online Nov. 18, 2021.
43. Waradon, et al. Nature Medicine. 2020; 26:681-687.
44. Ji, et al. J Clin Invest., 2021,1 131(13)
45. Oreste, et al. Mucosal Immunol. 2020, 26:1-12.
46. Matsumoto, et al. Front Microbiol., 2012, 16; 3:53.
47. Zhao, et al. Viruses. 2014, 6(2): 938-950.
48. Mori, et al. Bioorg Med Chem Lett. 2008, 18(14): 4249-4252
49. Paterson, et al., Science, 2005, 310:451-453.
50. Hsu, et al., Household and Personal Care, 2009, 33-36.
51. Chen et al. Handbook of Green Tea and Health Research. ISBN 978-1-60741-045-4 Editor: H. McKinley and M. Jamieson, Nova Science Publishers, Inc. 2009.
52. Steinmann et al., Br J Pharmacol, 2013, 168 1059-1073.
53. Colpitts C C, Schang L M. A small molecule inhibits virion attachment to heparan sulfate- or sialic acid-containing glycans. J Virol 88 (2014) 7806-7817.
54. Reid, et al., Antiviral Res, 2014, 109,171-174.
55. De Rossi S S, Thoppay J, Dickinson D P, Looney S, Stuart M, Ogbureke K U, Hsu S. A phase II clinical trial of a natural formulation containing tea catechins for xerostomia. Oral Surg, Oral Med Oral Pathol Oral Radiol. 2014,118(4):447-454.
56. Zhao M, Jiang J, Zheng R, Fu B, Pearl H, Dickinson D, and Hsu S. A proprietary topical preparation containing EGCG-stearate and glycerin with inhibitory effects on herpes simplex virus: case report. Inflammation & Allergy—Drug Targets, 11:364-8, 2012.
57. Zhao M, Zheng R, Jiang J, Dickinson D, Fu B, Chu T, Lee L, Pearl H, and Hsu S. Topical Lipophilic EGCG on Herpes Labialis: A Phase II Clinical Trial of AverTeaX. 0000, 120:717-724. 2015.
58. Chen P, Wang H, Du Q, Ito Y. Purification of long-chain fatty acid ester of epigallocatechin-3-O-gallate by high-speed counter-current chromatography. J Chromatogr A. 2002 Dec. 20; 982(1):163-5.
59. Chen P, Tan Y, Sun D, Zheng X M. A novel long-chain acyl-derivative of epigallocatechin-3-O-gallate prepared and purified from green tea polyphenols. J Zhejiang Univ Sci. 2003 November-December; 4(6):714-8.
60. Chen P, Sun D. A new catechin derivative as antioxidant for vegetable oil. Journal of the Chinese Cereals and oils association. 2003 18:5:77-78.
61. Sun D, Zhang S, Zhu Y, Wang Y. Study on antioxidant activity of lipid-soluble tea polyphenol in lipid. Cereals & Oil (Chinese). 2014 27:1:42
62. Ryu, S.; You, H. J.; Kim, Y. W.; Lee, A.; Ko, G. P.; Lee, S. J.; Song, M. J. Inactivation of norovirus and surrogates by natural phytochemicals and bioactive substances. Mol Nutr Food Res., 2015, 59(1), 65-74.
63. Hsu, S. Compounds derived from epigallocatechin-3-gallate (EGCG) as a novel approach to the prevention of viral infections. Inflammation & Allergy—Drug Target, 2015, 14, 13-18.
64. de Oliveira A, Adams S D, Lee L H, Murray S R, Hsu S D, Hammond J R, Dickinson D, Chen P, Chu T C. Inhibition of herpes simplex virus type 1 with the modified green tea polyphenol palmitoyl-epigallocatechin gallate. Food Chem Toxicol 52 (2012) 207-215.
65. Xu J, Xu Z, Zheng W. A Review of the Antiviral Role of Green Tea Catechins. Molecules. 2017 Aug. 12; 22(8). pii: E1337. doi: 10.3390/molecules22081337.
66. Ciesek, S.; von Hahn, T.; Colpitts, C. C.; Schang, L. M.; Friesland, M.; Steinmann, J.; Manns, M. P.; Ott, M.; Wedemeyer, H.; Meuleman, P.; Pietschmann, T.; Steinmann, E. The green tea polyphenol, epigallocatechin-3-gallate, inhibits hepatitis C virus entry. Hepatology. 2011, 54(6), 1947-55.
67. Calland, N.; Sahuc, M E.; Belouzard, S.; Pène, V.; Bonnafous, P.; Mesalam, A. A.; Deloison, G.; Descamps, V.; Sahpaz, S.; Wychowski, C.; Lambert, O.; Brodin, P.; Duverlie, G.; Meuleman, P.; Rosenberg, A. R.; Dubuisson, J.; Rouillé, Y.; Séron, K. Polyphenols Inhibit Hepatitis C Virus Entry by a New Mechanism of Action. J Virol., 2015, 89(19), 10053-63
68. Huang, HC.; Tao, M. H.; Hung, TM.; Chen, J. C.; Lin, Z. J.; Huang, C. (−)-Epigallocatechin-3-gallate inhibits entry of hepatitis B virus into hepatocytes. Antiviral Res., 2014, 111, 100-11.
69. Williamson, M. P.; McCormick, T. G.; Nance, C. L.; Shearer, W. T. Epigallocatechin gallate, the main polyphenol in green tea, binds to the T-cell receptor, CD4: Potential for HIV-1 therapy. J Allergy Clin Immunol., 2006, 118(6), 1369-74.
70. Kawai, K.; Tsuno, N. H.; Kitayama, J.; Okaji, Y.; Yazawa, K.; Asakage, M.; Hori, N.; Watanabe, T.; Takahashi, K.; Nagawa, H. Epigallocatechin gallate, the main component of tea polyphenol, binds to CD4 and interferes with gp120 binding. J Allergy Clin Immunol., 2003, 112(5), 951-7.
71. Nance, C. L.; Siwak, E. B.; Shearer, W. T. Preclinical development of the green tea catechin, epigallocatechin gallate, as an HIV-1 therapy. J Allergy Clin Immunol., 2009, 123(2), 459-65.
72. Huang J L, Yan C, Liu X W, et al. Progress of research on the effect of epigallocatechin-3-gallate against influenza virus. Chinese Journal of Virology, 2018; 34:2:259-263.
73. Kim M, Kim S Y, Lee H W, Shin J S, Kim P, Jung Y S, Jeong H S, Hyun J K, Lee C K. Inhibition of influenza virus internalization by (−)-epigallocatechin-3-gallate. Antiviral Res. 2013 November; 100(2):460-72.
74. Ge H, Liu G, Xiang Y F, Wang Y, Guo C W, Chen N H, Zhang Y J, Wang Y F, Kitazato K, Xu J. The mechanism of poly-galloyl-glucoses preventing Influenza A virus entry into host cells. PLOS One. 2014 Apr. 9; 9(4)
75. Koszalka P, Danielle Tilmanis D, and Hurt A. Influenza antivirals currently in late-phase clinical trial. Influenza Other Respir Viruses. 2017 May; 11(3): 240-246.
76. Song J M, Lee K H, Seong B L. Antiviral effect of catechins in green tea on influenza virus. Antiviral Res. 2005 November; 68(2):66-74.
77. Müller P, Downard K M. Catechin inhibition of influenza neuraminidase and its molecular basis with mass spectrometry. J Pharm Biomed Anal. 2015; 111:222-30.
78. Cho E J, Xia S, Ma L C, Robertus J, Krug R M, Anslyn E V, Montelione G T, Ellington A D. Identification of influenza virus inhibitors targeting NS1A utilizing fluorescence polarization-based high-throughput assay. J Biomol Screen. 2012 April; 17(4):448-59.
79. Kesic M J1, Simmons S O, Bauer R, Jaspers I. Nrf2 expression modifies influenza A entry and replication in nasal epithelial cells. Free Radic Biol Med. 2011 Jul. 15; 51(2):444-53.
80. Kuzuhara T, Iwai Y, Takahashi H, et al. Green tea catechins inhibit the endonuclease activity of influenza A virus RNA polymerase. PLOS Curr. 2009; 1:RRN1052.
81. Kowalinski E, Zubieta C, Wolkerstorfer A, et al. Structural analysis of specific metal chelating inhibitor binding to the endonuclease domain of influenza pH1N1 (2009) polymerase. PLOS Pathog. 2012; 8(8):e1002831.
82. Matthew J. Kesic, Megan Meyer, Rebecca Bauer, Ilona Jaspers. Exposure to Ozone Modulates Human Airway Protease/Antiprotease Balance Contributing to Increased Influenza A Infection, PLOS One. 2012; 7(4): e35108.
83. Matthew Zirui Tay, Chek Meng Poh, Laurent Rénia, Paul A. MacAry, Lisa F. P. Ng. The trinity of COVID-19: immunity, inflammation and intervention. Nat Rev Immunol. 2020 Apr. 28:1-12.
84. Changhyun Roh. A facile inhibitor screening of SARS coronavirus N protein using nanoparticle-based RNA oligonucleotide. Int J Nanomedicine. 2012; 7:2173-2179.
85. Sisi Kang, Mei Yang, Zhongsi Hong, Liping Zhang, Zhaoxia Huang, Xiaoxue Chen, Suhua He, Ziliang Zhou, Zhechong Zhou, Qiuyue Chen, Yan Yan, Changsheng Zhang, Hong Shan, Shoudeng Chen. Crystal structure of SARS-CoV-2 nucleocapsid protein RNA binding domain reveals potential unique drug targeting sites. Acta Pharm Sin B. 2020 Apr. 20.
86. Thi Thanh Hanh Nguyen, Hye-Jin Woo, Hee-Kyoung Kang, Van Dao Nguyen, Young-Min Kim, Do-Won Kim, Sul-Ah Ahn, Yongmei Xia, Doman Kim. Flavonoid-mediated inhibition of SARS coronavirus 3C-like protease expressed in Pichia pastoris. Biotechnol Lett. 2012; 34(5): 831-838.
87. Nilanjan Adhikari, Sandip K. Baidya, Achintya Saha, Tarun Jha. Structural Insight Into the Viral 3C-Like Protease Inhibitors: Comparative SAR/QSAR Approaches. Viral Proteases and Their Inhibitors. 2017:317-409.
88. Wei Y, Chen P, Ling T, et al. Certain (−)-epigallocatechin-3-gallate (EGCG) auto-oxidation products (EAOPs) retain the cytotoxic activities of EGCG. Food Chem. 2016; 204:218-226.
89. Jiarong Zhong, Douglas Dickinson, Lester Sampath, Stephen Hsu. Effects of Epigallocatechin-3-Gallate-Palmitate (EC16) on In Vitro Norovirus Infection. Microbiol Infect Dis. 2021; 5(5): 1-7.
90. Ping, C., Dickinson, D., Hsu, S. Lipid-soluble green tea polyphenols: potentials for health care and consumer products. Green Tea and Health Research. In Helen McKinley and Mark Jamieson (Eds.). Handbook of Green Tea and Health Research. 45-61. Nova Publishers 2009; 45-61.
91. Widjaja N, Dickinson D, Shao X, et al. Persistent Virucidal Activity in Novel Alcohol-Based Sanitizer Formulation (ProtecTeaV) for Potential Use Against Norovirus. Microbiology & Infectious Diseases, 2018; 2(1).
92. Dickinson D, Xayaraj S, Dickinson S, Shao X and Hsu S. Effect of Novel Formulations using Lipophilic Epigallocatechin-3-Gallate against Influenza Virus Infection. Microbiology & Infectious Diseases, 2018; 2(3): 1-8.
93. Guilherme Dias de Melo, Françoise Lazarini, Sylvain Levallois, Charlotte Hautefort, Vincent Michel, Florence Larrous, Benjamin Verillaud, Caroline Aparicio, Sebastien Wagner, Gilles Gheusi, Lauriane Kergoat, Etienne Kornobis, Flora Donati, Thomas Cokelaer, Rémi Hervochon, Yoann Madec, Emmanuel Roze, Dominique Salmon, Hervé Bourhy, Marc Lecuit, Pierre-Marie Lledo. COVID-19-related anosmia is associated with viral persistence and inflammation in human olfactory epithelium and brain infection in hamsters. Sci Transl Med. 2021 May 3.
94. Guilherme Dias de Melo, Françoise Lazarini, Sylvain Levallois, et al. COVID-19-related anosmia is associated with viral persistence and inflammation in human olfactory epithelium and brain infection in hamsters. Sci Transl Med. 2021 May 3: eabf8396.
95. Bertrand Bryche, Audrey St Albin, Severine Murri, Sandra Lacôthe, Coralie Pulido, Meriadeg Ar Gouilh, Sandrine Lesellier, Alexandre Servat, Marine Wasniewski, Evelyne Picard-Meyer, Elodie Monchatre-Leroy, Romain Volmer, Olivier Rampin, Ronan Le Goffic, Philippe Marianneau, Nicolas Meunier. Massive transient damage of the olfactory epithelium associated with infection of sustentacular cells by SARS-CoV-2 in golden Syrian hamsters. Brain Behav Immun. 2020 October; 89:579-586.
96. Dongyan Zhou, Jasper Fuk-Woo Chan, Biao Zhou, et al. Robust SARS-CoV-2 infection in nasal turbinates after treatment with systemic neutralizing antibodies. Cell Host Microbe. 2021 Apr. 14; 29(4): 551-563.e5.
97. Sang Hyun Ra, Joon Seo Lim, Gwang-un Kim, Min Jae Kim, Jiwon Jung, Sung-Han Kim. Upper respiratory viral load in asymptomatic individuals and mildly symptomatic patients with SARS-CoV-2 infection. Thorax 2021; 76:61-63.
98. Contributing Authors and Panelists, Gary Goldenberg, Maida Taylor, Brian Berman, Mark Kaufmann, William Abramovits, Joshua Zeichner. Sinecatechins Ointment, 15% for the Treatment of External Genital and Perianal Warts: Proceedings of an Expert Panel Roundtable Meeting. J Clin Aesthet Dermatol. 2016 March; 9(3 Suppl 1): S2-S15.
99. Zhou F, Xu S, Shen T, et al. Antioxidant effects of lipophilic tea polyphenols on diethylnitrosamine/phenobarbital-induced hepatocarcinogenesis in rats. In Vivo, 2014; 28:495-503.
100. Shen T, Xu S, Zhou F, et al. Chemoprevention by lipid-soluble tea polyphenols in DEN/PB induced hepatic precancerous lesions. Anticancer Research. 2014; 34(2):683-93.
101. USFDA GRAS Notice 772: GRAS Notice for Oil-Soluble Green Tea Extract (Green Tea Catechin Palmitate). 2019. PDF: https://www.fda.gov/media/126906/download. FDA “no question (approval)” letter: https://www.fda.gov/media/120299/download
102. Xueling Shao, Lu Wang, Sydney W. Gu, Tinchun Chu, Stephen Hsu. Antiviral Effect of Extract from Fagopyrum buckwheats Against Two Nonenveloped Viruses. Microbiology & Infectious Diseases. 2020, 4(3): 1-8.
103. Pervin M, Unno K, Takagaki A, Isemura M, Nakamura Y. Function of Green Tea Catechins in the Brain: Epigallocatechin Gallate and its Metabolites. Int J Mol Sci. 2019 Jul. 25; 20(15).
104. Lin L C, Wang M N, Tseng T Y, Sung J S, Tsai T H. Pharmacokinetics of (−)-epigallocatechin-3-gallate in conscious and freely moving rats and its brain regional distribution. J Agric Food Chem. 2007 Feb. 21; 55(4):1517-24.
105. de la Torre, R.; de Sola, S.; Farré, M.; Xicota, L.; Cuenca-Royo, A.; Rodriguez, J.; León, A.; Langohr, K.; Gomis-González, M.; Hernandez, G.; et al. A phase 1, randomized double-blind, placebo controlled trial to evaluate safety and efficacy of epigallocatechin-3-gallate and cognitive training in adults with Fragile X syndrome. Clin. Nutr. 2019.
106. Singh N A, Mandal A K, Khan Z A. Potential neuroprotective properties of epigallocatechin-3-gallate (EGCG). Nutr J. 2016 Jun. 7; 15(1):60. doi: 10.1186/s12937-016-0179-4.
107. Brett L. Hurst, Douglas Dickinson, Stephen Hsu. Epigallocatechin-3-Gallate (EGCG) Inhibits Sars-CoV-2 Infection in Primate Epithelial Cells. Microbiol Infect Dis. 2021; 5(2): 1-6.
108. Reed L J, Muench H. A simple method of estimating fifty percent endpoints. Am J Epidemiol. 1938; 27:493-7.
109. Minsu Jang, Rackhyun Park, Yea-In Park, Yeo-Eun Cha, Ayane Yamamoto, Jin I. Lee, and Junsoo Park. EGCG, a green tea polyphenol, inhibits human coronavirus replication in vitro. Biochem Biophys Res Commun. 2021 Apr. 2; 547:23-28.
110. Mutsuo Yamaya, Hidekazu Nishimura, Xue Deng, Mitsuru Sugawara, Oshi Watanabe, Kazuhiro Nomura, Yoshitaka Shimotai, Haruki Momma, Masakazu Ichinose, and Tetsuaki Kawaseh. Inhibitory effects of glycopyrronium, formoterol, and budesonide on coronavirus HCoV-229E replication and cytokine production by primary cultures of human nasal and tracheal epithelial cells. Respir Investig. 2020 May; 58(3): 155-168.
111. Mahnaz Ramezanpour, Harrison Bolt, Karen Hon, George Spyro Bouras, Alkis James Psaltis, Peter-John Wormald, Sarah Vreugde. Cytokine-Induced Modulation of SARS-CoV 2 Receptor Expression in Primary Human Nasal Epithelial Cells. Pathogens. 2021 July; 10(7): 848.
112. Gamage A M, Tan K S, Chan W O Y, Liu J, Tan C W, Ong Y K, et al. (2020) Infection of human Nasal Epithelial Cells with SARS-CoV-2 and a 382-nt deletion isolate lacking ORF8 reveals similar viral kinetics and host transcriptional profiles. PLOS Pathog 16(12): e1009130. https://doi.org/10.1371/journal.ppat.1009130.
113. M Wang, R Cao, L Zhang, X Yang, J Liu, M Xu, Z Shi, Hu Z, Zhong W, Xiao G. Remdesivir and chloroquine effectively inhibit the recently emerged novel coronavirus (2019-nCoV) in vitro. Cell Res 30, 269-271 (2020).

1. A pharmaceutical formulation for nasal administration, comprising:
nanoparticles of a green tea catechin derivative; and
a pharmaceutically acceptable carrier,
optionally wherein the green tea catechin derivative is a derivative of epigallocatechin-3-gallate, epicatechin, epigallocatechin, or epicatechin-3-gallate, or a combination thereof.

2.-11. (canceled)

12. The pharmaceutical formulation of claim 1, wherein the green tea catechin derivative is an epigallocatechin-3-gallate-palmitate, preferably wherein the green tea catechin derivative has the structure of:

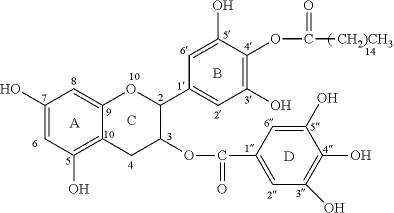

13. The pharmaceutical formulation of claim 1, wherein the nanoparticles have:
(a) a median diameter of less than 500 nm, less than 400 nm, less than 300 nm, or less than 200 nm, such as ranging from about 10 nm to about 500 nm, from about 10 nm to about 400 nm, from about 10 nm to about 300 nm, from about 10 nm to about 200 nm, from about 10 nm to about 150 nm, from about 50 nm to about 500 nm, from about 50 nm to about 400 nm, from about 50 nm to about 300 nm, from about 50 nm to about 200 nm, or from about 50 nm to about 150 nm; and/or
(b) a zeta potential in a range from about-80 mV to about-10 mV or from about-50 mV to about-20 mV.

14. The pharmaceutical formulation of claim 1, wherein the green tea catechin derivative is present in an amount from about 0.01% (w/v) to about 2% (w/v), from about 0.01% (w/v) to about 1% (w/v), from about 0.01% (w/v) to about 0.5% (w/v), from about 0.01% to about 0.25% (w/v), or from about 0.01% (w/v) to about 0.1% (w/v) of the pharmaceutical formulation.

15. (canceled)

16. The pharmaceutical formulation of claim 1, wherein the pharmaceutically acceptable carrier is a mixture of (a) glycerol and (b) water or an aqueous solution, and optionally (c) an alcohol, and optionally wherein the glycerol is present in an amount from 0.1 wt % to 99.5 wt %, from 0.1 wt % to 35 wt %, from 10 wt % to 99.5 wt % or from 40 wt % to 99.5 wt %; or the glycerol is present in an amount from 0.1 wt % to 99.5 wt %, from 0.1 wt % to 35 wt %, from 10 wt % to 99.5 wt %, or from 40 wt % to 99.5 wt % in the pharmaceutical formulation.

17. The pharmaceutical formulation of claim 16, wherein the water or aqueous solution is present in an amount from 0.1 wt % to 99 wt %, from 60 wt % to 99 wt %, from 0.1 wt % to 30 wt %, or from 0.1 wt % to 15 wt % in the pharmaceutical formulation.

18. The pharmaceutical formulation of claim 16, wherein the alcohol, when present, is:
(a) in an amount ≤1 wt %, such as from about 0.1 wt % to about 0.3 wt %, in the pharmaceutical formulation; and/or
(b) ethanol, n-propanol, or isopropanol, or a combination thereof.

19. The pharmaceutical formulation of claim 16, wherein the aqueous solution is a saline solution (normal, isotonic, or hypertonic), phosphate buffered saline, a citrate buffer solution, MEM medium, or Hanks balanced salt solution, such as a normal saline solution.

20. (canceled)

21. The pharmaceutical formulation of claim 1, having a pH in a range from 3 to 8.5, from 3 to 7.4, from 4 to 7, from 4 to 6, from 4 to 5, from 5 to 7, from 5 to 6, or from 6.5 to 7.4.

22. (canceled)

23. The pharmaceutical formulation of claim 1, further comprising a dispersing agent, optionally wherein the dispersing agent is a carbohydrate or salt thereof (e.g., sodium carboxymethyl cellulose), or a metaphosphate (e.g., trimetaphosphate, hexametaphosphate, etc.) or salt thereof (e.g., sodium trimetaphosphate, sodium hexametaphosphate, etc.), or a combination thereof, and optionally wherein the dispersing agent is sodium hexametaphosphate.

24. The pharmaceutical formulation of claim 23, wherein the dispersing agent is present in an amount from about 0.0005% (w/v) to about 1% (w/v), from about 0.005% (w/v) to about 1% (w/v), from about 0.05% (w/v) to about 1% (w/v), from about 0.005% (w/v) to about 0.5% (w/v), from about 0.01% (w/v) to about 0.5% (w/v), from about 0.1% (w/v) to about 0.5% (w/v), from about 0.005% (w/v) to about 2% (w/v), from about 0.01% (w/v) to about 2% (w/v), or from about 0.1% (w/v) to about 2% (w/v) in the pharmaceutical formulation.

25. (canceled)

26. The pharmaceutical formulation of claim 1, further comprising an emulsifier, an emollient, a buffering agent, a thickening agent, a chelating agent, or a preservative, or a combination thereof.

27. (canceled)

28. A delivery device comprising an inhaler and the pharmaceutical formulation of claim 1, optionally wherein the inhaler is a pressurized metered dose inhaler that is configured to deliver a unit dosage of the pharmaceutical formulation per puff.

29.-30. (canceled)

31. A kit comprising one or more nasal swabs and the pharmaceutical formulation of claim 1.

32-34. (canceled)

35. A method of making the pharmaceutical formulation of claim 1, comprising:
(i) dissolving the green tea catechin derivative in a first pharmaceutically acceptable carrier to form a green tea catechin derivative solution;
(ii) mixing the green tea catechin derivative solution with a second pharmaceutically acceptable carrier to produce a suspension formulation comprising nanoparticles of the green tea catechin derivative; and
optionally (iii) mixing the suspension formulation with a third pharmaceutically acceptable carrier.

36. (canceled)

37. The method of claim 35, wherein:
(a) the first pharmaceutically acceptable carrier is an alcohol or a water mixture thereof; the second pharmaceutically acceptable carrier is glycerol or a water mixture thereof; the third pharmaceutically acceptable carrier is water or an aqueous solution; and/or
(b) the aqueous solution comprises a dispersing agent, such as a metaphosphate.

38.-59. (canceled)

60. A method for treating anosmia in a subject in need thereof, comprising:
(i) administering the pharmaceutical formulation of claim 1 to the nasal vestibule or passages of the subject,
optionally wherein the subject was exposed to, is exposed to, or is expected to be exposed to an individual having one or more symptoms of COVID infection, such as with fever, cough, shortness of breath, diarrhea, sneezing, runny nose, and/or sore throat.

61. A method for treating long COVID in a subject in need thereof, comprising:
(i) administering the pharmaceutical formulation of claim 1 to the nasal vestibule or passages of the subject,
optionally wherein the long COVID comprises one or more symptoms selected from the group consisting of headache, persistent loss of smell (i.e. anosmia), persistent loss of taste, memory loss, brain fog (difficulty concentrating, sense of confusion or disorientation), dizziness, anxiety, depression, earache, hearing loss, ringing in ears (tinnitus), fatigue, and gastrointestinal issues, or a combination thereof.

62.-68. (canceled)

69. The method of claim 60, wherein following the administration or all of the administrations, the subject regain at least a portion of the sense of smell, compared to the subject before the administration or first administration.

70.-71. (canceled)

72. A method for reducing viral infection, comprising administering the pharmaceutical formulation of claim 1 to a subject in need thereof,
optionally wherein the subject has not been exposed to the virus, or has been exposed to the virus, or is infected with the virus, and
optionally wherein the virus is a respiratory virus.

73.-79. (canceled)


SEQUENCE LISTING (1 sequence)

SEQ ID NO: 1 - protein, 1273 aa, Human coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT